SEQUENCE LISTING (74 sequences)

SEQ ID NO: 1 - protein, 31 aa, Artificial Sequence
  AGP Peptide
DLSKQMEEEAVRLFIEWLKNGGPSSGAPPPS

SEQ ID NO: 2 - protein, 30 aa, Artificial Sequence
  AGP peptide of AGP-FPP
HAEGTFTSDVSSYLEGQAAKEFIAWLVKGR

SEQ ID NO: 3 - protein, 39 aa, Artificial Sequence
  AGP peptide of AGP-FPP
HGEGTFTSDLSKQMEEEAVRLFIEWLKNGGPSSGAPPPS

SEQ ID NO: 4 - protein, 38 aa, Artificial Sequence
  AGP peptide of AGP-FPP
GEGTFTWELSKQMEEEAVRLFIEWLKNGGPSSGAPPPS

SEQ ID NO: 5 - protein, 38 aa, Artificial Sequence
  AGP peptide of AGP-FPP
GEGTFTSQLSKQMEEEAVRLFIEWLKNGGPSSGAPPPS

SEQ ID NO: 6 - protein, 41 aa, Artificial Sequence
  AGP peptide of AGP-FPP
KRHSDGTFTSDLSKQMEEEAVRLFIEWLKNGGPSSGAPPPS

SEQ ID NO: 7 - protein, 37 aa, Artificial Sequence
  AGP peptide of AGP-FPP
HSDGTFSDLSKGMEEEAVRLHEWLKNGGPSSGAPPPS

SEQ ID NO: 8 - protein, 39 aa, Artificial Sequence; identical to SEQ ID NO: 3
  AGP peptide of AGP-FPP

SEQ ID NO: 9 - protein, 30 aa, Artificial Sequence
  AGP peptide of AGP-FPP
HAEGTFTSKVSSYLEGQAAKEFIAWLVKGR

SEQ ID NO: 10 - protein, 38 aa, Artificial Sequence
  AGP peptide of AGP-FPP
GEGTFTSDLSKQMEEEAVRLFIEWLKNGGPSSGAPPPS

SEQ ID NO: 11 - protein, 37 aa, Artificial Sequence
  AGP peptide of AGP-FPP
EGTFTSDLSKQMEEEAVRLFIEWLKNGGPSSGAPPPS

SEQ ID NO: 12 - protein, 36 aa, Artificial Sequence
  AGP peptide of AGP-FPP
GTFTSDLSKQMEEEAVRLFIEWLKNGGPSSGAPPPS

SEQ ID NO: 13 - protein, 87 aa, Artificial Sequence
  antagonist fragment of AGP-FPP
MKIILWLCVFGLFLATLFPVSWQMPVESGLSSEDSASSESFASKIKRHSDGTFTSDLSKQMEEEAVRLFIEWLKNGGPSSGAPPPSG

SEQ ID NO: 14 - protein, 32 aa, Artificial Sequence
  AGP peptide of AGP-FPP
SDLSKQMEEEAVRLFIEWLKNGGPSSGAPPPS

SEQ ID NO: 15 - protein, 35 aa, Artificial Sequence
  AGP peptide of AGP-FPP
TFTSDLSKQMEEEAVRLFIEWLKNGGPSSGAPPPS

SEQ ID NO: 16 - protein, 34 aa, Artificial Sequence
  AGP peptide of AGP-FPP
FTSDLSKQMEEEAVRLFIEWLKNGGPSSGAPPPS

SEQ ID NO: 17 - protein, 33 aa, Artificial Sequence
  AGP peptide of AGP-FPP
TSDLSKQMEEEAVRLFIEWLKNGGPSSGAPPPS

SEQ ID NO: 18 - protein, 38 aa, Artificial Sequence
  AGP peptide of AGP-FPP
GEGTFTSELSKQMEEEAVRLFIEWLKNGGPSSGAPPPS

SEQ ID NO: 19 - protein, 847 aa, Artificial Sequence
  Antagonist of GLP-Receptor Fusion Proteins
DLSKQMEEEAVRLFIEWLKNGGPSSGAPPPSGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 20 - protein, 906 aa, Artificial Sequence
  Antagonist of GLP-Receptor Fusion Proteins
DLSKQMEEEAVRLFIEWLKNGGPSSGAPPPSGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 21 - protein, 636 aa, Artificial Sequence
  Antagonist of GLP-Receptor Fusion Proteins
DLSKQMEEEAVRLFIEWLKNGGPSSGAPPPSVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 22 - protein, 847 aa, Artificial Sequence
  Antagonist of GLP-Receptor Fusion Proteins
HAEGTFTSDVSSYLEGQAAKEFIAAWLVKGRGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 23 - protein, 906 aa, Artificial Sequence
  Antagonist of GLP-Receptor Fusion Proteins
HAEGTFTSDVSSYLEGQAAKEFIAAWLVKGRGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 24 - protein, 636 aa, Artificial Sequence
  Antagonist of GLP-Receptor Fusion Proteins
HAEGTFTSDVSSYLEGQAAKEFIAAWLVKGRVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 25 - protein, 855 aa, Artificial Sequence
  Antagonist of GLP-Receptor Fusion Proteins
HGEGTFTSDLSKQMEEEAVRLFIEWLKNGGPSSGAPPPSGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 26 - protein, 914 aa, Artificial Sequence
  Antagonist of GLP-Receptor Fusion Proteins
HGEGTFTSDLSKQMEEEAVRLFIEWLKNGGPSSGAPPPSGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 27 - protein, 644 aa, Artificial Sequence
  Antagonist of GLP-Receptor Fusion Proteins
HGEGTFTSDLSKQMEEEAVRLFIEWLKNGGPSSGAPPPSVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 28 - protein, 854 aa, Artificial Sequence
  Antagonist of GLP-Receptor Fusion Proteins
GEGTFTWELSKQMEEEAVRLFIEWLKNGGPSSGAPPPSGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 29 - protein, 913 aa, Artificial Sequence
  Antagonist of GLP-Receptor Fusion Proteins
GEGTFTWELSKQMEEEAVRLFIEWLKNGGPSSGAPPPSGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 30 - protein, 643 aa, Artificial Sequence
  Antagonist of GLP-Receptor Fusion Proteins
GEGTFTWELSKQMEEEAVRLFIEWLKNGGPSSGAPPPSVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 31 - protein, 854 aa, Artificial Sequence
  Antagonist of GLP-Receptor Fusion Proteins
GEGTFTSQLSKQMEEEAVRLFIEWLKNGGPSSGAPPPSGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 32 - protein, 913 aa, Artificial Sequence
  Antagonist of GLP-Receptor Fusion Proteins
GEGTFTSQLSKQMEEEAVRLFIEWLKNGGPSSGAPPPSGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 33 - protein, 643 aa, Artificial Sequence
  Antagonist of GLP-Receptor Fusion Proteins
GEGTFTSQLSKQMEEEAVRLFIEWLKNGGPSSGAPPPSVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 34 - protein, 857 aa, Artificial Sequence
  Antagonist of GLP-Receptor Fusion Proteins
KRHSDGTFTSDLSKQMEEEAVRLFIEWLKNGGPSSGAPPPSGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 35 - protein, 916 aa, Artificial Sequence
  Antagonist of GLP-Receptor Fusion Proteins
KRHSDGTFTSDLSKQMEEEAVRLFIEWLKNGGPSSGAPPPSGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 36 - protein, 646 aa, Artificial Sequence
  Antagonist of GLP-Receptor Fusion Proteins
KRHSDGTFTSDLSKQMEEEAVRLFIEWLKNGGPSSGAPPPSVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 37 - protein, 853 aa, Artificial Sequence
  Antagonist of GLP-Receptor Fusion Proteins
HSDGTFSDLSKGMEEEAVRLHEWLKNGGPSSGAPPPSGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 38 - protein, 912 aa, Artificial Sequence
  Antagonist of GLP-Receptor Fusion Proteins
HSDGTFSDLSKGMEEEAVRLHEWLKNGGPSSGAPPPSGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 39 - protein, 642 aa, Artificial Sequence
  Antagonist of GLP-Receptor Fusion Proteins
HSDGTFSDLSKGMEEEAVRLHEWLKNGGPSSGAPPPSVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 40 - protein, 855 aa, Artificial Sequence; identical to SEQ ID NO: 25
  Antagonist of GLP-Receptor Fusion Proteins

SEQ ID NO: 41 - protein, 914 aa, Artificial Sequence; identical to SEQ ID NO: 26
  Antagonist of GLP-Receptor Fusion Proteins

SEQ ID NO: 42 - protein, 644 aa, Artificial Sequence; identical to SEQ ID NO: 27
  Antagonist of GLP-Receptor Fusion Proteins

SEQ ID NO: 43 - protein, 846 aa, Artificial Sequence
  Antagonist of GLP-Receptor Fusion Proteins
HAEGTFTSKVSSYLEGQAAKEFIAWLVKGRGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 44 - protein, 905 aa, Artificial Sequence
  Antagonist of GLP-Receptor Fusion Proteins
HAEGTFTSKVSSYLEGQAAKEFIAWLVKGRGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 45 - protein, 635 aa, Artificial Sequence
  Antagonist of GLP-Receptor Fusion Proteins
HAEGTFTSKVSSYLEGQAAKEFIAWLVKGRVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 46 - protein, 616 aa, Artificial Sequence
  Antagonist of GLP-Receptor Fusion Proteins
DLSKQMEEEAVRLFIEWLKNGGPSSGAPPPSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 47 - protein, 616 aa, Artificial Sequence
  Antagonist of GLP-Receptor Fusion Proteins
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLDLSKQMEEEAVRLFIEWLKNGGPSSGAPPPS

SEQ ID NO: 48 - protein, 620 aa, Artificial Sequence
  Antagonist of GLP-Receptor Fusion Proteins
TFTSDLSKQMEEEAVRLFIEWLKNGGPSSGAPPPSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 49 - protein, 623 aa, Artificial Sequence
  Antagonist of GLP-Receptor Fusion Proteins
GEGTFTSELSKQMEEEAVRLFIEWLKNGGPSSGAPPPSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 50 - protein, 851 aa, Artificial Sequence
  Antagonist of GLP-Receptor Fusion Proteins
TFTSDLSKQMEEEAVRLFIEWLKNGGPSSGAPPPSGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 51 - protein, 910 aa, Artificial Sequence
  Antagonist of GLP-Receptor Fusion Proteins
TFTSDLSKQMEEEAVRLFIEWLKNGGPSSGAPPPSGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 52 - protein, 640 aa, Artificial Sequence
  Antagonist of GLP-Receptor Fusion Proteins
TFTSDLSKQMEEEAVRLFIEWLKNGGPSSGAPPPSVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 53 - protein, 854 aa, Artificial Sequence
  Antagonist of GLP-Receptor Fusion Proteins
GEGTFTSELSKQMEEEAVRLFIEWLKNGGPSSGAPPPSGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 54 - protein, 913 aa, Artificial Sequence
  Antagonist of GLP-Receptor Fusion Proteins
GEGTFTSELSKQMEEEAVRLFIEWLKNGGPSSGAPPPSGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 55 - protein, 643 aa, Artificial Sequence
  Antagonist of GLP-Receptor Fusion Proteins
GEGTFTSELSKQMEEEAVRLFIEWLKNGGPSSGAPPPSVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 56 - protein, 679 aa, Artificial Sequence
  FPP peptide of AGP-FPP
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 57 - protein, 585 aa, Artificial Sequence [sequence omitted]

SEQ ID NO: 58 - protein, 816 aa, Artificial Sequence
  FPP peptide of AGP-FPP
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 59 - protein, 875 aa, Artificial Sequence
  FPP peptide of AGP-FPP
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGGSPAGSPTSTEEGSSTPSGATGSPGSSPSASTGTGPGASPGTSSTSPGTSESATPESGPGTSTEPSEGSAPGTSTEPSEGSAP

SEQ ID NO: 60 - protein, 605 aa, Artificial Sequence
  FPP peptide of AGP-FPP
VPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 61 - protein, 864 aa, Artificial Sequence
  Fusion Protein Partner
GSETATSGSETAGTSESATSESGAGSTAGSETSTEAGTSESATSESGAGSETATSGSETAGSETATSGSETAGTSTEASEGSASGTSTEASEGSASGTSESATSESGAGSETATSGSETAGTSTEASEGSASGSTAGSETSTEAGTSESATSESGAGTSESATSESGAGSETATSGSETAGTSESATSESGAGTSTEASEGSASGSETATSGSETAGSETATSGSETAGTSTEASEGSASGSTAGSETSTEAGTSESATSESGAGTSTEASEGSASGSETATSGSETAGSTAGSETSTEAGSTAGSETSTEAGSETATSGSETAGTSESATSESGAGTSESATSESGAGSETATSGSETAGTSESATSESGAGTSESATSESGAGSETATSGSETAGSETATSGSETAGTSTEASEGSASGSTAGSETSTEAGSETATSGSETAGTSESATSESGAGSTAGSETSTEAGSTAGSETSTEAGSTAGSETSTEAGTSTEASEGSASGSTAGSETSTEAGSTAGSETSTEAGTSTEASEGSASGSTAGSETSTEAGSETATSGSETAGTSTEASEGSASGTSESATSESGAGSETATSGSETAGTSESATSESGAGTSESATSESGAGSETATSGSETAGTSESATSESGAGSETATSGSETAGTSTEASEGSASGTSTEASEGSASGSTAGSETSTEAGSTAGSETSTEAGSETATSGSETAGTSESATSESGAGTSESATSESGAGSETATSGSETAGSETATSGSETAGSETATSGSETAGTSTEASEGSASGTSESATSESGAGSETATSGSETAGSETATSGSETAGTSESATSESGAGTSESATSESGAGSETATSGSETA

SEQ ID NO: 62 - protein, 864 aa, Artificial Sequence
  Fusion Protein Partner
GTSTEPSEPGSAGTSTEPSEPGSAGSEPATSGTEPSGSGASEPTSTEPGSEPATSGTEPSGSEPATSGTEPSGSEPATSGTEPSGSGASEPTSTEPGTSTEPSEPGSAGSEPATSGTEPSGTSTEPSEPGSAGSEPATSGTEPSGSEPATSGTEPSGTSTEPSEPGSAGTSTEPSEPGSAGSEPATSGTEPSGSEPATSGTEPSGTSEPSTSEPGAGSGASEPTSTEPGTSEPSTSEPGAGSEPATSGTEPSGSEPATSGTEPSGTSTEPSEPGSAGTSTEPSEPGSAGSGASEPTSTEPGSEPATSGTEPSGSEPATSGTEPSGSEPATSGTEPSGSEPATSGTEPSGTSTEPSEPGSAGSEPATSGTEPSGSGASEPTSTEPGTSTEPSEPGSAGSEPATSGTEPSGSGASEPTSTEPGTSTEPSEPGSAGSGASEPTSTEPGSEPATSGTEPSGSGASEPTSTEPGSEPATSGTEPSGSGASEPTSTEPGTSTEPSEPGSAGSEPATSGTEPSGSGASEPTSTEPGTSTEPSEPGSAGSEPATSGTEPSGTSTEPSEPGSAGSEPATSGTEPSGTSTEPSEPGSAGTSTEPSEPGSAGTSTEPSEPGSAGTSTEPSEPGSAGTSTEPSEPGSAGTSTEPSEPGSAGTSEPSTSEPGAGSGASEPTSTEPGTSTEPSEPGSAGTSTEPSEPGSAGTSTEPSEPGSAGSEPATSGTEPSGSGASEPTSTEPGSEPATSGTEPSGSEPATSGTEPSGSEPATSGTEPSGSEPATSGTEPSGTSEPSTSEPGAGSEPATSGTEPSGSGASEPTSTEPGTSTEPSEPGSAGSEPATSGTEPSGSGASEPTSTEPGTSTEPSEPGSA

SEQ ID NO: 63 - protein, 1318 aa, Artificial Sequence
  Fusion Protein Partner
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGPEPTGPAPSGGSEPATSGSETPGTSESATPESGPGSPAGSPTSTEEGTSESATPESGPGSPAGSPTSTEEGSPAGSPTSTEEGTSESATPESGPGSPAGSPTSTEEGSPAGSPTSTEEGSTSSTAESPGPGSTSESPSGTAPGTSPSGESSTAPGSTSESPSGTAPGSTSESPSGTAPGTSPSGESSTAPGTSTEPSEGSAPGTSESATPESGPGTSESATPESGPGSEPATSGSETPGTSESATPESGPGTSESATPESGPGTSTEPSEGSAPGTSESATPESGPGTSTEPSEGSAPGTSPSGESSTAPGTSPSGESSTAPGTSPSGESSTAPGTSTEPSEGSAPGSPAGSPTSTEEGTSTEPSEGSAPGSSPSASTGTGPGSSTPSGATGSPGSSTPSGATGSPGSSTPSGATGSPGSSTPSGATGSPGASPGTSSTGSPGASASGAPSTGGTSPSGESSTAPGSTSSTAESPGPGTSPSGESSTAPGTSESATPESGPGTSTEPSEGSAPGTSTEPSEGSAPGSSPSASTGTGPGSSTPSGATGSPGASPGTSSTGSPGTSTPESGSASPGTSPSGESSTAPGTSPSGESSTAPGTSESATPESGPGSEPATSGSETPGTSTEPSEGSAPGSTSESPSGTAPGSTSESPSGTAPGTSTPESGSASPGSPAGSPTSTEEGTSESATPESGPGTSTEPSEGSAPGSPAGSPTSTEEGTSESATPESGPGSEPATSGSETPGSSTPSGATGSPGASPGTSSTGSPGSSTPSGATGSPGSTSESPSGTAPGTSPSGESSTAPGSTSSTAESPGPGSSTPSGATGSPGASPGTSSTGSPGTPGSGTASSSPGSPAGSPTSTEEGSPAGSPTSTEEGTSTEPSEGSAP

SEQ ID NO: 64 - protein, 924 aa, Artificial Sequence
  Fusion Protein Partner
MAEPAGSPTSTEEGASPGTSSTGSPGSSTPSGATGSPGSSTPSGATGSPGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAP

SEQ ID NO: 65 - protein, 913 aa, Artificial Sequence
  Fusion Protein Partner
MAEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 66 - protein, 864 aa, Artificial Sequence
  Fusion Protein Partner
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 67 - protein, 875 aa, Artificial Sequence
  Fusion Protein Partner
GSTSESPSGTAPGTSPSGESSTAPGSTSESPSGTAPGSTSESPSGTAPGTSTPESGSASPGTSTPESGSASPGSTSESPSGTAPGSTSESPSGTAPGTSPSGESSTAPGSTSESPSGTAPGTSPSGESSTAPGTSPSGESSTAPGSTSSTAESPGPGTSPSGESSTAPGTSPSGESSTAPGSTSSTAESPGPGTSTPESGSASPGTSTPESGSASPGSTSESPSGTAPGSTSESPSGTAPGTSTPESGSASPGSTSSTAESPGPGTSTPESGSASPGSTSESPSGTAPGTSPSGESSTAPGSTSSTAESPGPGTSPSGESSTAPGTSTPESGSASPGSTSSTAESPGPGSTSSTAESPGPGSTSSTAESPGPGSTSSTAESPGPGTSPSGESSTAPGSTSESPSGTAPGSTSESPSGTAPGTSTPESGPXXXGASASGAPSTXXXXSESPSGTAPGSTSESPSGTAPGSTSESPSGTAPGSTSESPSGTAPGSTSESPSGTAPGSTSESPSGTAPGTSTPESGSASPGTSPSGESSTAPGTSPSGESSTAPGSTSSTAESPGPGTSPSGESSTAPGTSTPESGSASPGSTSESPSGTAPGSTSESPSGTAPGTSPSGESSTAPGSTSESPSGTAPGTSTPESGSASPGTSTPESGSASPGSTSESPSGTAPGTSTPESGSASPGSTSSTAESPGPGSTSESPSGTAPGSTSESPSGTAPGTSPSGESSTAPGSTSSTAESPGPGTSPSGESSTAPGTSTPESGSASPGTSPSGESSTAPGTSPSGESSTAPGTSPSGESSTAPGSTSSTAESPGPGSTSSTAESPGPGTSPSGESSTAPGSSPSASTGTGPGSSTPSGATGSPGSSTPSGATGSP

SEQ ID NO: 68 - protein, 836 aa, Artificial Sequence
  Fusion Protein Partner
GSSESGSSEGGPGSSESGSSEGGPGESPGGSSGSESGSGGEPSESGSSGESPGGSSGSESGESPGGSSGSESGSSESGSSEGGPGSSESGSSEGGPGSSESGSSEGGPGESPGGSSGSESGESPGGSSGSESGESPGGSSGSESGSSESGSSEGGPGSSESGSSEGGPGSSESGSSEGGPGSSESGSSEGGPGSSESGSSEGGPGSSESGSSEGGPGSGGEPSESGSSGESPGGSSGSESGESPGGSSGSESGSGGEPSESGSSGSEGSSGPGESSGSSESGSSEGGPGSGGEPSESGSSGSEGSSGPGESSGSSESGSSEGGPGSGGEPSESGSSGESPGGSSGSESGSGGEPSESGSSGSGGEPSESGSSGSSESGSSEGGPGSGGEPSESGSSGSGGEPSESGSSGSEGSSGPGESSGESPGGSSGSESGSEGSSGPGESSGSEGSSGPGESSGSGGEPSESGSSGSSESGSSEGGPGSSESGSSEGGPGESPGGSSGSESGSGGEPSESGSSGSEGSSGPGESSGESPGGSSGSESGSEGSSGPGSSESGSSEGGPGSGGEPSESGSSGSEGSSGPGESSGSEGSSGPGESSGSEGSSGPGESSGSGGEPSESGSSGSGGEPSESGSSGESPGGSSGSESGESPGGSSGSESGSGGEPSESGSSGSEGSSGPGESSGESPGGSSGSESGSSESGSSEGGPGSSESGSSEGGPGSSESGSSEGGPGSGGEPSESGSSGSSESGSSEGGPGESPGGSSGSESGSGGEPSESGSSGSSESGSSEGGPGESPGGSSGSESGSGGEPSESGSSGESPGGSSGSESGSGGEPSESGSS

SEQ ID NO: 69 - protein, 906 aa, Artificial Sequence; identical to SEQ ID NO: 20
  Fusion Protein Partner

SEQ ID NO: 70 - protein, 640 aa, Artificial Sequence
  Fusion Protein Partner
ESGPGSPAGSPTSTEEGSPAGSPTSTEEGSPAGSPTSTEEGTSESATPESGPGTSTEPSEGSAPGSTSSTAESPGPGSTSSTAESPGPGSTSESPSGTAPGSTSSTAESPGPGSTSSTAESPGPGTSTPESGSASPGSTSESPSGTAPGTSPSGESSTAPGSTSESPSGTAPGSTSESPSGTAPGTSPSGESSTAPGSTSESPSGTAPGSTSESPSGTAPGTSPSGESSTAPGSTSESPSGTAPGSTSESPSGTAPGSTSESPSGTAPGTSTPESGSASPGSTSESPSGTAPGTSTPESGSASPGSTSSTAESPGPGSTSSTAESPGPGTSTPESGSASPGTSTPESGSASPGSTSESPSGTAPGTSTPESGSASPGTSTPESGSASPGSTSESPSGTAPGSTSESPSGTAPGSTSESPSGTAPGSTSSTAESPGPGTSTPESGSASPGTSTPESGSASPGSTSESPSGTAPGSTSESPSGTAPGTSTPESGSASPGSTSESPSGTAPGSTSESPSGTAPGTSTPESGSASPGTSPSGESSTAPGSTSSTAESPGPGTSPSGESSTAPGSTSSTAESPGPGTSTPESGSASPGSTSESPSGTAPGSTSSTAESPGPGTSTPESGSASPGTSTPESGSASP

SEQ ID NO: 71 - protein, 847 aa, Artificial Sequence; identical to SEQ ID NO: 22
  Fusion Protein Partner

SEQ ID NO: 72 - protein, 576 aa, Artificial Sequence
  Fusion Protein Partner
GSSESGSSEGGPGSGGEPSESGSSGSSESGSSEGGPGSSESGSSEGGPGSSESGSSEGGPGSSESGSSEGGPGSSESGSSEGGPGESPGGSSGSESGSEGSSGPGESSGSSESGSSEGGPGSSESGSSEGGPGSSESGSSEGGPGSGGEPSESGSSGESPGGSSGSESGESPGGSSGSESGSGGEPSESGSSGSSESGSSEGGPGSGGEPSESGSSGSGGEPSESGSSGSEGSSGPGESSGESPGGSSGSESGSGGEPSESGSSGSGGEPSESGSSGSGGEPSESGSSGSSESGSSEGGPGESPGGSSGSESGESPGGSSGSESGESPGGSSGSESGESPGGSSGSESGESPGGSSGSESGSSESGSSEGGPGSGGEPSESGSSGSEGSSGPGESSGSSESGSSEGGPGSGGEPSESGSSGSSESGSSEGGPGSGGEPSESGSSGESPGGSSGSESGESPGGSSGSESGSSESGSSEGGPGSGGEPSESGSSGSSESGSSEGGPGSGGEPSESGSSGSGGEPSESGSSGESPGGSSGSESGSEGSSGPGESSGSSESGSSEGGPGSEGSSGPGESS

SEQ ID NO: 73 - protein, 540 aa, Artificial Sequence
  Fusion Protein Partner
GSTSSTAESPGPGSTSSTAESPGPGSTSESPSGTAPGSTSSTAESPGPGSTSSTAESPGPGTSTPESGSASPGSTSESPSGTAPGTSPSGESSTAPGSTSESPSGTAPGSTSESPSGTAPGTSPSGESSTAPGSTSESPSGTAPGSTSESPSGTAPGTSPSGESSTAPGSTSESPSGTAPGSTSESPSGTAPGSTSESPSGTAPGTSTPESGSASPGSTSESPSGTAPGTSTPESGSASPGSTSSTAESPGPGSTSSTAESPGPGTSTPESGSASPGTSTPESGSASPGSTSESPSGTAPGTSTPESGSASPGTSTPESGSASPGSTSESPSGTAPGSTSESPSGTAPGSTSESPSGTAPGSTSSTAESPGPGTSTPESGSASPGTSTPESGSASPGSTSESPSGTAPGSTSESPSGTAPGTSTPESGSASPGSTSESPSGTAPGSTSESPSGTAPGTSTPESGSASPGTSPSGESSTAPGSTSSTAESPGPGTSPSGESSTAPGSTSSTAESPGPGTSTPESGSASPGSTSESPSGTAP

SEQ ID NO: 74 - protein, 504 aa, Artificial Sequence
  Fusion Protein Partner
GASPGTSSTGSPGSSPSASTGTGPGSSPSASTGTGPGTPGSGTASSSPGSSTPSGATGSPGSXPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSXPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSP

(19) United States
(12) Patent Application Publication
(10) Pub. No.: US 2015/0005233 A1
(43) Pub. Date: Jan. 1, 2015

(54) TREATMENT OF HYPOGLYCEMIA

(71) Applicant: Seneb Biosciences, Inc., North Wales, PA (US)

(72) Inventor: Shawn DeFrees, North Wales, PA (US)

(73) Assignee: Seneb Biosciences, Inc., North Wales, PA (US)

(21) Appl. No.: 14/377,475

(22) PCT Filed: Feb. 8, 2013
(86) PCT No.: PCT/US13/25442

§ 371 Date: Aug. 7, 2014

Related U.S. Application Data
(60) Provisional application No. 61/596,627, filed on Feb. 8, 2012.

(51) Int. Cl. C07K 14/605

(52) U.S. Cl. CPC C07K 14/605; C07K 2319/31; C07K 2319/95
USPC 514/6.7; 530/399; 514/11.7; 435/69.7; 536/23.4; 514/6.8

(57) ABSTRACT

This invention provides compounds, compositions, and methods for treating hypogly.

### CROSS-REFERENCE TO RELATED APPLICATIONS

This application claims priority to U.S. Provisional Application No. 61/596,627, filed on Feb. 8, 2012, which is incorporated herein in its entirety for all purposes.

### FIELD OF THE INVENTION

This invention relates to methods of treating and ameliorating hyperinsulinemia, hypoglycemia and hyperinsulinemia with hypoglycemia comprising the step of administering an antagonist of the Glucagon-Like Peptide (GLP-1) receptor (AGP)-fusion protein, e.g. GLP-1 fragment or analogue thereof.

### BACKGROUND OF THE INVENTION

Congenital hyperinsulinism (CHI, OMIM 256450) is a genetic disorder of pancreatic β-cell function characterized by failure to suppress insulin secretion in the presence of hypoglycemia, resulting in brain damage or death if inadequately treated. Germline mutations in several genes have been associated with congenital hyperinsulinism and include, for example, the sulfonylurea receptor (SUR-1, encoded by ABCC8), an inward rectifying potassium channel (Kir6.2, encoded by KCNJ11), glucokinase (GCK), glutamate dehydrogenase (GLUD-1), short-chain L-3-hydroxyacyl-CoA (SCHAD, encoded by HADSC) and mitochondrial uncoupling protein 2 (UCP2). In approximately 40% of the cases, the genetic cause of the condition has not been characterized. Loss-of-function mutations in the K_{ATP} channel (composed by two subunits: Kir6.2 and SUR-1) may be responsible for the most common and severe form of congenital hyperinsulinism (K_{ATP} HI), with many patients requiring near total pancreatectomy to control hypoglycemia, leading to long hospital stays and life threatening complications.

Post-prandial hypoglycemia is a frequent complication of Nissen fundoplication (e.g. in children), a procedure commonly performed to treat severe gastroesophageal reflux. Up to 30% of patients undergoing this procedure develop dumping syndrome. Dumping syndrome is characterized by early symptoms or “early dumping” due to the fluid shifts provoked by the osmotic load in the small bowel and “late dumping” or post-prandial hypoglycemia. Post-prandial hypoglycemia can also be caused by gastric bypass surgery for obesity.

Neuroendocrine tumors including such cancers as insulinoma, hepatomas, mesotheliaoma and fibrosarcoma cause hyperinsulinemia accompanied by hypoglycemia. In addition, insulinoma can be a single solid tumor, microadenomatosis or islet cell hyperplasia (nesidioblastosis). Surgery is the treatment of choice for insulinoma after use of, for example, endoscopic ultrasonography to locate the tumor. Current therapy for an insulinoma if the tumor cannot be located in the pancreas is stepwise pancreatectomy (from tail to head). Resection is stopped with an 85% pancreatectomy, even if the tumor is not found, to avoid a malabsorption problem. As many as 15% of patients have persistent hypoglycemia, even after surgical resection of the pancreas. Additionally, postoperative complications may include acute pancreatitis, peritonitis, fistulas, pseudocyst formation and diabetes mellitus. For those patients that remain hypoglycemic after surgery, are awaiting surgery or are not eligible for surgery, agents are needed to control blood sugar levels and improve complications resulting from the disease.

Hypoglycemia may result from other genetic diseases that include Beckwith-Wiedemann syndrome and congenital disorders of glycosylation. Beckwith-Wiedemann syndrome is characterized by mutation in genes NSDI, H19, KCNQ10T1 and CDKN1C and causes hypoglycemia. Congenital disorders of glycosylation are a family of genetic diseases characterized by mutations in one or more glycosyltransferases and include types 1a-n, types IIa-o and type I/IIx.

Administration of insulin, alcohol and sulfonylureas can cause hypoglycemia. Hypoglycemia can result from non-insulin secreting mesenchymal tumor and end-stage liver or renal disease. A non-insulin-secreting mesenchymal tumor may also cause hypoglycemia because of secretion of insulin-like growth factor (IGF) that mimics insulin. Renal disease can cause hypoglycemia with and without associated hyperinsulinemia. The underlying cause of renal disease can be genetic or non-genetic. Genetic conditions include polycystic kidney disease (e.g. PKD1, ARPKD, PKD2, PKD3, PKDTS). Hypoglycemia is caused by dialysis and medications used to treat kidney disease.

Effective treatments for hypoglycemia, hyperinsulinemia, hypoglycemia with hyperinsulinemia are urgently needed.

### BRIEF SUMMARY OF THE INVENTION

The present disclosure is directed to compositions and methods that can be useful for or the treatment of any disease, disorder or condition that is improved, ameliorated, or inhibited by the administration of an antagonist of a Glucagon-Like Peptide-1 (GLP-1) receptor (AGP) fusion protein, e.g. GLP-1 fragment or analogue thereof. In particular, the present invention provides compositions of fusion proteins comprising one or more extended recombinant polypeptides linked to an antagonist of GLP-1 receptor. In part, the present disclosure is directed to pharmaceutical compositions comprising the fusion proteins and the uses thereof for treating glucose regulating peptide-related diseases, disorders or conditions.

In one embodiment the invention provides an isolated fusion protein, comprising the AGP of an AGP-FPP fusion protein that is at least about 90%, or about 95%, or about 96%, or about 97%, or about 98%, or about 99% identical to an amino acid sequence selected from Table 1, wherein said antagonist to GLP-1 receptor is linked to an recombinant polypeptide that is about 90%, or about 95%, or about 96%, or about 97%, or about 98%, or about 99% identical to an amino acid sequence selected from Table 2.

In one embodiment, the isolated fusion protein is less immunogenic compared to the AGP not linked to the fusion protein partner (FPP), wherein immunogenicity is ascertained by, e.g., measuring production of IgG antigodies selectively binding to the biologically active protein after administration of comparable doses to a subject.

In some embodiments, the AGP-fusion proteins exhibit enhanced pharmacokinetic properties compared to AGP no linked to a fusion protein, wherein theh enhanced properties include but are not limited to longer terminall half-life, larger are under the curve, increased time in which the blood concentrationremains within the therapeutic window, increased time between consecutive doses, and decreased dose in moles over time. IN some embodiments, the terminal half-life of the AGP-fusion protein administered to a subject is increased at least about two fold, or at least about three-fold, or at least about four-fold, or at least about five-fold, or at least about six-fold, or at least about seven-fold, or at least about eight-fold, or at least about nine-fold, or at least about ten-fold, or at least about 20-fold, or at least about 40-fold, or at least about 60-fold, or at least about 100-fold compared to AGP not linked to a fusion protein and administered to a subject at a comparable dose. In other embodiments, the enhanced pharmacokinetic property is reflected by the fact that the blood concentrations that remain within the therapeutic window for the AGP-fusion protein for a given period are at least about two fold, or at least about three-fold, or at least about four-fold, or at least about five-fold, or at least about six-fold, or at least about seven-fold, or at least about eight-fold, or at least about nine-fold, or at least about ten-fold, or at least about 20-fold, or at least about 40-fold, or at least about 60-fold, or at least about 100-fold compared to AGP not linked to a fusion protein and administered to a subject at a comparable dose. The increase in half-life and time spent within the therapeutic window permits less frequent dosing and decreased amounts of the fusion protein (in moles equivalent) that are administered to a subject, compared to the corresponding AGP not linked to a fusion protein. In one embodiment, the therapeutically effective dose regimen results in a gain in time of at least two fold, or at least about three-fold, or at least about four-fold, or at least about five-fold, or at least about six-fold, or at least about seven-fold, or at least about eight-fold, or at least about nine-fold, or at least about ten-fold, or at least about 20-fold, or at least about 40-fold, or at least about 60-fold, or at least about 100-fold between at least two consecutive Cₘₐₓ peaks and/or Cₘᵢₙ troughs for blood levels of the fusion protein and administered using a comparable dose regimen to a subject.

In an exemplary embodiment, the FPP (fusion protein partner) subunit alters the pharmacokinetic profile of the AGP protein to which it is conjugated, a scenario similar to PEGylation of a protein, but in such a way that the underlying biological activity of AGP remains essentially unchanged by the conjugation of the FPP.

In exemplary embodiments, administration of the AGP protein of the invention raises the blood glucose AUC of a hypoglycemic patient at least 30 mmol·min/L, at least 40 mmol·min/L, at least 50 mmol·min/L, at least 60 mmol·min/L, at least 70 mmol·min/L, at least 80 mmol·min/L, at least 90 mmol·min/L, at least 100 mmol·min/L, at least 110 mmol·min/L, at least 120 mmol·min/L, at least 130 mmol·min/L, at least 140 mmol·min/L, at least 150 mmol·min/L, at least 160 mmol·min/L, at least 170 mmol·min/L, at least 180 mmol·min/L, at least 190 mmol·min/L, at least 200 mmol·min/L as compared with the blood glucose level of the patient at a time point prior to administration of the composition of the invention. In some embodiment, administration of the AGP protein of the invention raises the blood glucose AUC of a hypoglycemic patient from about 30-200 mmol·min/L, from about 40-190 mmol·min/L, from about 50-180 mmol·min/L, from about 60-170 mmol·min/L, from about 70-160 mmol·min/L, from about 80-150 mmol·min/L, from about 90-140 mmol·min/L, from about 100-130 mmol·min/L, from about 110-120 mmol·min/L as compared with the blood glucose level of the patient at a time point prior to administration of the composition of the invention.

In some embodiments, administration of the AGP protein of the invention reduces the insulin-to-glucose AUC ratio of a hypoglycemic patient by 0.5-1.0, by 1.0-1.5, by 1.5-2.0, by 2.0-2.5, by 2.5-3.0, by at least 0.5, by at least 1.0, by at least 1.5, by at least 2.0, by 0.2-4.0, by 0.5-3.5, by 1.0-3.0, by 1.5-2.5, by at least 2.5, by at least 3.0, by at least 3.5, by at least 4.0 as compared with the insulin-to-glucose AUC ratio of the patient at a time point prior to administration of the composition of the invention.

In some embodiments, treatment with the AGP protein of the invention inhibits (AAM) amino acid-stimulated insulin secretion in islets isolated from hypoglycemic patients, e.g. human K_{ATP}HI patients, and cultured in standard medium, e.g. RPMI-1640 medium containing 10 mmol/L glucose. Details of an exemplary protocol for an islet assay are provided in Example 12 of the instant specification.

In still other embodiments, treatment with the AGP protein raises fasting blood glucose levels in SUR-1^{-/-} mice by 5-30 mg/dl, by 10-25 mg/dl, by 15-20 mg/dl, by at least 5 mg/dl, by at least 10 mg/dl, by at least 15 mg/dl, by at least 20 mg/dl, by at least 25 mg/dl, by at least 25 mg/dl, by at least 30 mg/dl, by at least 35 mg/dl, by at least 40 mg/dl, by at least 45 mg/dl, by 10-30 mg/dl, by 20-30 mg/dl as compared with the fasting blood glucose level of the mice at a time point prior to administration of the composition of the invention.

In yet other embodiments, treatment with the AGP protein decreases basal intracellular cAMP in SUR-1^{-/-} islets isolated from SUR-1^{-/-} mice and cultured in standard medium, e.g. RPMI 1640 medium containing 10 mM glucose, and/or reduces the amino acid-stimulated increase in cAMP in SUR-1^{-/-} islets isolated from SUR-1^{-/-} mice and/or reduces the baseline insulin secretion by SUR-1^{-/-} islets isolated from SUR-1^{-/-} mice and/or reduces the amino acid-stimulated insulin secretion by SUR-1^{-/-} islets isolated from SUR-1^{-/-} mice. Details of an exemplary protocol for the islet assays are provided in Example 11 of the instant specification.

In some embodiments, the antagonist to the GLP-1 receptor and the FPP are linked via a spacer, wherein the spacer sequence comprises between about 1 to about 50 amino acid residues that optionally comprises a cleavage sequence. In one embodiment, the cleavage sequence is susceptible to cleavage by a protease. Non-limiting examples of such protease include FXIa, FXIIA, kallikrein, FVIIa, FIXa, FXa, thrombin, elastase-2, granzyme B, MMP-2, MMP13, MMP17 or MMP20, TEV, enterokinase, rhinovirus 3C protease, and sortase A.

In some embodiments, the isolated fusion protein is configured to have reduced binding affinity for a target receptor of the corresponding AGP, as compared to the corresponding AGP not linked to FPP. In one embodiment, the AGP-FPP exhibits binding affinity for a target receptor of the AGP in the range of about 0.01%-30%, or about 0.1% to about 20%, or about 1% to about 15%, or about 2% to about 10% of the binding affinity of the corresponding AGP that lacks the fusion protein. In another embodiment, the AGP-fusion protein exhibits binding affinity for a target receptor of the AGP that is reduced at least about 3-fold, or at least about 5-fold, or at least about 6-fold, or at least about 7-fold, or at least about 8-fold, or at least about 9-fold, or at least about 10-fold, or at least about 12-fold, or at least about 15-fold, or at least about 17-fold, or at least about 20-fold, or at least about 30-fold, or at least about 50-fold or at least about 100-fold less binding affinity compared to AGP not linked to a fusion protein. In a related embodiment, a fusion protein with reduced affinity can have reduced receptor-mediated clearance and a corresponding increase in half-life of at least about 3-fold, or at least about four-fold, or at least about five-fold, or at least about six-fold, or at least about seven-fold, or at least about eight-fold, or at least about nine-fold, or at least about ten-fold, or at least about 20-fold, or at least about 40-fold, or at least about 60-fold, or at least about 100-fold longer compared to the corresponding AGP not linked to a fusion protein.

In one embodiment, the invention provides an isolated AGP-FPP comprising an amino acids sequence that has at least about 80%, or at least about 90%, or at least about 91%, or at least about 92%, or at least about 93%, or at least about 94%, or at least about 95%, or at least about 96%, or at least about 97%, or at least about 98%, or at least about 99%, or 100% sequence identity to a sequence selected from Table 3.

In some embodiments, the invention provides AGP-fusion proteins wherein the AGP-fusion protein exhibits increased solubility of at least three-fold, or at least about four-fold, or at least about five-fold, or at least about six-fold, or at least about seven-fold, or at least about eight-fold, or at least about nine-fold, or at least about ten-fold, or at least about 15-fold, or at least a 20-fold, or at least 40-fold, or at least 60-fold at physiologic conditions compared to the GP not linked to the fusion protein.

In some embodiments, AGP-FPP exhibit an increased apparent molecular weight as determined by size exclusion chromatography, compared to the actual molecular weight, wherein the apparent molecular weight is at least about 100 kD, or at least about 150 kD, or at least about 200 kD, or at least about 300 kD, or at least about 400 kD, or at least about 500 kD, or at least about 600 kD, or at least about 700 kD, while the actual molecular weight of each GP component of the fusion protein is less than about 25 kD. Accordingly, the AGP-fusion proteins can have an Apparent Molecular Weight that is about 4-fold greater, or about 5-fold greater, or about 6-fold greater, or about 7-fold greater, or about 8-fold greater than the actual molecular weight of the fusion protein. In some cases, the isolated AGP-fusion protein of the foregoing embodiments exhibits an apparent molecular weight factor under physiologic conditions that is greater than about 4, or about 5, or about 6, or about 7, or about 8.

The invention contemplates AGP-FPP compositions comprising, but not limited to AGP selected from Table 1 (or fragments or sequence variants thereof), fusion protein partners selected from Table 2 (or sequence variants thereof) that are in a configuration selected from Table 3. Generally, the resulting AGP-fusion protein will retain at least a portion of the biological activity of the corresponding AGP not linked to the fusion protein. In other cases, the AGP component either becomes biologically active or has an increase in activity upon its release from the fusion protein by cleavage of an optional cleavage sequence incorporated within spacer sequences into the AGP-fusion protein.

In one embodiment of the AGP-fusion protein composition, the invention provides a fusion protein of formula I:

(FPP)ₓ-AGP-(FPP)_{y}

wherein independently for each occurrence, AGP is a is a antagonist of GLP-3 receptor; x is either 0 or 1 and y is either 0 or 1 wherein x+y≦1; and FPP is an recombinant polypeptide.

In some embodiments, the FPP is fused to an antagonist of the GLP-1 receptor on an N- or C-terminus of the AGP.

In another embodiment of the AGP-FPP composition, the invention provides a fusion protein of formula II:

(FPP)ₓ-(AGP)-(S)_{y}-(FPP)_{y}

wherein independently for each occurrence, AGP is an antagonist of the GLP-1 receptor; S is a spacer sequence having between 1 to about 50 amino acid residues that can optionally include a cleavage sequence; x is either 0 or 1 and y is either 0 or 1 wherein x+y≦1; and FPP is a recombinant polypeptide.

In another embodiment, the invention provides an isolated fusion protein, wherein the fusion protein is of formula III:

(AGP)-(S)ₓ-(FPP)-(S)_{y}-(AGP)-(S)_{z}-(FPP)_{z}

wherein independently for each occurrence, AGP is an antagonist of the GLP-1 receptor; S is a spacer sequence having between 1 to about 50 amino acid residues that can optionally include a cleavage sequence; x is either 0 or 1; y is either 0 or 1; z is either 0 or 1; and FPP is a recombinant polypeptide.

In another embodiment, the invention provides an isolated fusion protein, wherein the fusion protein is of formula IV:

(FPP)ₓ-(S)_{y}-(AGP)-(S)_{z}-(FPP)-(AGP)

wherein independently for each occurrence, AGP is an antagonist of the GLP-1 receptor; S is a spacer sequence having between 1 to about 50 amino acid residues that can optionally include a cleavage sequence; x is either 0 or 1; y is either 0 or 1; z is either 0 or 1; and FPP is a recombinant polypeptide.

In another embodiment, the invention provides an isolated fusion glucose regulating peptide, wherein the fusion protein is of formula V:

(AGP)ₓ-(S)ₓ-(AGP)-(S)_{y}-(FPP)

wherein independently for each occurrence, AGP is an antagonist of the GLP-1 receptor; S is a spacer sequence having between 1 to about 50 amino acid residues that can optionally include a cleavage sequence; x is either 0 or 1; y is either 0 or 1; and FPP is a recombinant polypeptide.

In another embodiment, the invention provides an isolated fusion protein, wherein the fusion protein is of formula VI:

(FPP)-(S)ₓ-(AGP)-(S)_{y}-(AGP)

wherein independently for each occurrence, AGP is an antagonist of the GLP-1 receptor; S is a spacer sequence having between 1 to about 50 amino acid residues that can optionally include a cleavage sequence; x is either 0 or 1; y is either 0 or 1; and FPP is a recombinant polypeptide.

In another embodiment, the invention provides an isolated fusion protein, wherein the fusion protein is of formula VII:

(FPP)-(S)ₓ-(AGP)-(S)_{y}-(AGP)-(FPP)

wherein independently for each occurrence, AGP is an antagonist of the GLP-1 receptor; S is a spacer sequence having between 1 to about 50 amino acid residues that can optionally include a cleavage sequence; x is either 0 or 1; y is either 0 or 1; and FPP is a recombinant polypeptide.

In another embodiment, the invention provides an isolated fusion protein, wherein the fusion protein is of formula VIII:

((S)ₘ-(AGP)ₓ-(S)ₙ-(FPP)_{y}-(S)ₒ)ₜ

wherein t is an integer that is greater than 0 (1, 2, 3, etc.); independently each of m, n, o, x, and y is an integer (0, 1, 2, 3, etc.), AGP is an antagonist of the GLP-1 receptor; S is an spacer, optionally comprising a cleavage site; and FPP is a recombinant polypeptide, with the proviso that: (1) x+y>1, (2) when t=1, x>0 and y>0, (3) when there is more than one AGP, S, or FPP, each AGP, FPP, or S are the same or are independently different; and (4) when t>1, each m, n, o, x, or y within each subunit are the same or are independently different.

In some embodiments, administration of a therapeutically effective dose of a fusion protein of an embodiment of formulas I-VIII to a subject in need thereof can result in a gain in time of at least two-fold, or at least three-fold, or at least four-fold, or at least five-fold or more spent within a therapeutic window for the fusion protein compared to the corresponding AGP not linked to the FPP of and administered at a comparable dose to a subject. In other cases, administration of a therapeutically effective dose of a fusion protein of an embodiment of formulas I-VIII to a subject in need thereof can result in a gain in time between consecutive doses necessary to maintain a therapeutically effective dose regimen of at least 48 h, or at least 72 h, or at least about 96 h, or at least about 120 h, or at least about 7 days, or at least about 14 days, or at least about 21 days between consecutive doses compared to a AGP not linked to FPP and administered at a comparable dose.

The fusion proteins can be designed to have different configurations, N- to C-terminus, of a AGP, FPP, and optional spacer sequences, including but not limited to FPP-AGP, AGP-FPP, FPP-S-AGP, AGP-S-FPP, FPP-AGP-FPP, AGP-AGP-FPP, FPP-AGP-AGP, AGP-S-AGP-FPP, FPP-AGP-S-AGP, and multimers thereof. The choice of configuration can, as disclosed herein, confer particular pharmacokinetic, physicochemical, or pharmacologic properties.

In some embodiments, the isolated fusion protein is characterized in that: (i) it has a longer half-life compared to the corresponding AGP that lacks the FPP; (ii) when a smaller molar amount of the fusion protein is administered to a subject in comparison to the corresponding AGP that lacks the FPP administered to a subject under an otherwise equivalent dose regimen, the fusion protein achieves a comparable area under the curve (AUC) as the corresponding AGP that lacks the FPP; (iii) when a smaller molar amount of the fusion protein is administered to a subject in comparison to the corresponding AGP that lacks the FPP administered to a subject under an otherwise equivalent dose regimen, the fusion protein achieves a comparable therapeutic effect as the corresponding AGP that lacks the FPP; (iv) when the fusion protein is administered to a subject less frequently in comparison to the corresponding AGP that lacks the FPP administered to a subject using an otherwise equivalent molar amount, the fusion protein achieves a comparable area under the curve (AUC) as the corresponding AGP that lacks the FPP; (v) when the fusion protein is administered to a subject less frequently in comparison to the corresponding AGP that lacks the FPP administered to a subject using an otherwise equivalent molar amount, the fusion protein achieves a comparable therapeutic effect as the corresponding AGP that lacks the FPP; (vi) when an accumulatively smaller molar amount of the fusion protein is administered to a subject in comparison to the corresponding AGP that lacks the FPP administered to a subject under an otherwise equivalent dose period, the fusion protein achieves comparable area under the curve (AUC) as the corresponding AGP that lacks the FPP; or (vii) when an accumulatively smaller molar amount of the fusion protein is administered to a subject in comparison to the corresponding AGP that lacks the FPP administered to a subject under an otherwise equivalent dose period, the fusion protein achieves comparable therapeutic effect as the corresponding AGP that lacks the FPP.

In one embodiment, the AGP-FPP described above exhibit a biological activity of at least about 0.1%, or at least about 1%, or at least about 2%, or at least about 3%, or at least about 4%, or at least about 5%, or at least about 10%, or at least about 20%, or at least about 30%, or at least 40%, or at least about 50%, or at least about 60%, or at least about 70%, or at least about 80%, or at least about 90%, or at least about 95% of the biological activity compared to the AGP not linked to FPP. In another embodiment, the AGP-FPP bind the same receptors as the corresponding parental AGP that is not covalently linked to FPP.

The invention provides a method of producing a fusion protein comprising an AGP fused to one or more recombinant polypeptides (FPP), comprising: (a) providing host cell comprising a recombinant polynucleotide molecule encoding the fusion protein (b) culturing the host cell under conditions permitting the expression of the fusion protein; and (c) recovering the fusion protein. In one embodiment of the method, the AGP of the fusion protein has at least 90% sequence identity to human AGP or a sequence selected from Table 1. In another embodiment of the method, the one or more FPP of the expressed fusion protein has at least about 90%, or about 91%, or about 92%, or about 93%, or about 94%, or about 95%, or about 96%, or about 97%, or about 98%, or about 99% to about 100% sequence identity to a sequence selected from Table 2. In another embodiment of the method, the polynucleotide encoding the FPP is codon optimized for enhanced expression of said fusion protein in the host cell. In another embodiment of the method, the host cell is a prokaryotic cell. In another embodiment of the method, the host cell is E. coli. In another embodiment of the method the isolated fusion protein is recovered from the host cell cytoplasm in substantially soluble form. In another embodiment, the E. coli strain is Origami® or Shuffle®.

The invention provides isolated nucleic acids comprising a polynucleotide sequence selected from (a) a polynucleotide encoding the fusion protein of any of the foregoing embodiments, or (b) the complement of the polynucleotide of (a). The invention provides expression vectors comprising the nucleic acid of any of the embodiments hereinabove described in this paragraph. In one embodiment, the expression vector of the foregoing further comprises a recombinant regulatory sequence operably linked to the polynucleotide sequence. In another embodiment, the polynucleotide sequence of the expression vectors of the foregoing is fused in frame to a polynucleotide encoding a secretion signal sequence, which can be a prokaryotic signal sequence. In one embodiment, the secretion signal sequence is selected from OmpA, DsbA, and PhoA signal sequences.

The invention provides a host cell, which can comprise an expression vector disclosed in the foregoing paragraph. In one embodiment, the host cell is a prokaryotic cell. In another embodiment, the host cell is E. coli. In another embodiment, the host cell is a eukaryotic cell.

In one embodiment, the invention provides pharmaceutical compositions comprising the fusion protein of any of the foregoing embodiments and a pharmaceutically acceptable carrier. In another embodiment, the invention provides kits, comprising packaging material and at least a first container comprising the pharmaceutical composition of the foregoing embodiment and a label identifying the pharmaceutical composition and storage and handling conditions, and a sheet of instructions for the reconstitution and/or administration of the pharmaceutical compositions to a subject.

The invention provides a method of treating an antagonist of GLP-1 receptor-related condition in a subject, comprising administering to the subject a therapeutically effective amount of the AGP-FPP of any of the foregoing embodiments. In one embodiment of the method, the antagonist of GLP-1 receptor related condition is selected from, but not limited to neonatal hyperinsulinism, congential hyperinsulinism, acute hypoglycemia, nocturnal hypoglycemia, chronic hypoglycemia, Beckwith-Wiedemann syndrome, congenital disorders of glycosylation, hypoglycemia resulting from dialysis, glucagonomas, secretory disorders of the airway, arthritis, neuroendocrine tumors, osteoporosis, central nervous system disease, restenosis, neurodegenerative disease, renal failure, congestive heart failure, nephrotic syndrome, cirrhosis, pulmonary edema, hypertension, and disorders wherein the reduction of food intake is desired, stroke, irritable bowel syndrome, myocardial infarction (e.g., reducing the morbidity and/or mortality associated therewith), stroke, acute coronary syndrome (e.g., characterized by an absence of Q-wave) myocardial infarction, post-surgical catabolic changes, hibernating myocardium or diabetic cardiomyopathy, post-prandial hypoglycemia, insufficient urinary sodium excretion, excessive urinary potassium concentration, conditions or disorders associated with toxic hypervolemia, (e.g., renal failure, congestive heart failure, nephrotic syndrome, cirrhosis, pulmonary edema, and hypertension), polycystic ovary syndrome, respiratory distress, nephropathy, left ventricular systolic dysfunction, (e.g., with abnormal left ventricular ejection fraction), gastrointestinal disorders such as diarrhea, postoperative dumping syndrome and irritable bowel syndrome, (i.e., via inhibition of antro-duodenal motility), critical illness polyneuropathy (CIPN), dyslipidemia, organ tissue injury caused by reperfusion of blood flow following ischemia, and coronary heart disease risk factor (CHDRF) syndrome, and any other indication for which AGP can be utilized.

In some embodiments, the composition can be administered subcutaneously, intramuscularly, or intravenously. In one embodiment, the composition is administered at a therapeutically effective amount. In one embodiment, the therapeutically effective amount results in a gain in time spent within a therapeutic window for the fusion protein compared to the corresponding AGP of the fusion protein not linked to the fusion protein and administered at a comparable dose to a subject. The gain in time spent within the therapeutic window can at least three-fold longer than the corresponding AGP not linked to the fusion protein, or alternatively, at least four-fold, or five-fold, or six-fold, or seven-fold, or eight-fold, or nine-fold, or at least 10-fold, or at least 20-fold, or at least about 30-fold, or at least about 50-fold, or at least about 100-fold longer than the corresponding AGP not linked to the fusion protein. In some embodiments of the method of treatment, (i) a smaller molar amount of (e.g. of about two-fold less, or about three-fold less, or about four-fold less, or about five-fold less, or about six-fold less, or about eight-fold less, or about 100 fold-less or greater) the fusion protein is administered in comparison to the corresponding AGP that lacks the FPP under an otherwise same dose regimen, and the fusion protein achieves a comparable area under the curve and/or a comparable therapeutic effect as the corresponding AGP that lacks the FPP; (ii) the fusion protein is administered less frequently (e.g., every two days, about every seven days, about every 14 days, about every 21 days, or about, monthly) in comparison to the corresponding AGP that lacks the FPP under an otherwise same dose amount, and the fusion protein achieves a comparable area under the curve and/or a comparable therapeutic effect as the corresponding AGP that lacks the FPP; or (iii) an accumulative smaller molar amount (e.g. about 5%, or about 10%, or about 20%, or about 40%, or about 50%, or about 60%, or about 70%, or about 80%, or about 90% less) of the fusion protein is administered in comparison to the corresponding AGP that lacks the FPP under the otherwise same dose regimen the fusion protein achieves a comparable area under the curve and/or a comparable therapeutic effect as the corresponding AGP that lacks the FPP. The accumulative smaller molar amount is measure for a period of at least about one week, or about 14 days, or about 21 days, or about one month. In some embodiments of the method, the therapeutic effect is a measured parameter selected from HbAlc concentrations, insulin concentrations, stimulated C peptide, fasting plasma glucose (FPG), serum cytokine levels, CRP levels, insulin secretion and Insulin-sensitivity index derived from an oral glucose tolerance test (OGTT), body weight, and food consumption.

In one embodiment, the present invention provides a method of treating a subject with congenital hyperinsulinism, comprising the step of administering to the subject an antagonist of the GLP-1 receptor fusion protein, thereby treating a subject with a congenital hyperinsulinism.

In another embodiment, the present invention provides a method of reducing an incidence of hypoglycemia in a subject with congenital hyperinsulinism, comprising the step of administering to the subject an antagonist of the GLP-1 receptor-fusion protein, thereby reducing an incidence of hypoglycemia in a subject with congenital hyperinsulinism.

In another embodiment, the present invention provides a method of ameliorating a congenital hyperinsulinism in a subject, comprising the step of administering to the subject an antagonist of the GLP-1 receptor fusion protein, thereby ameliorating a congenital hyperinsulinism in a subject.

In another embodiment, the present invention provides a method of inhibiting a development of a post-prandial hypoglycemia in a subject, comprising the step of administering to the subject an antagonist of the GLP-1 receptor fusion protein, thereby inhibiting the development of post-prandial hypoglycemia in a subject.

In another embodiment, the present invention provides a method of treating a subject with post-prandial hypoglycemia, comprising the step of administering to the subject an antagonist of the GLP-1 receptor fusion protein, thereby treating a subject with a post-prandial hypoglycemia.

In another embodiment, the present invention provides a method of reducing an incidence of a post-prandial hypoglycemia in a subject, comprising the step of administering to the subject an antagonist of the GLP-1 receptor fusion protein, thereby reducing an incidence of a post-prandial hypoglycemia in a subject.

In another embodiment, the present invention provides a method of ameliorating a post-prandial hypoglycemia in a subject, comprising the step of administering to the subject an antagonist of the GLP-1 receptor fusion protein, thereby ameliorating a post-prandial hypoglycemia in a subject.

In another embodiment, the present invention provides a method of inhibiting a development of a post-prandial hypoglycemia in a subject, comprising the step of administering to the subject an AGP-FPP polypeptide, thereby inhibiting a development of a post-prandial hypoglycemia in a subject.

In another embodiment, the present invention provides a method of treating a subject with a neonatal HI, comprising the step of administering to the subject an antagonist of the GLP-1 receptor fusion protein as a fusion protein, thereby treating a subject with a neonatal HI.

In another embodiment, the present invention provides a method of reducing an incidence of hypoglycemia in a neonate with neonatal HI, comprising the step of administering to the subject an antagonist of the GLP-1 receptor as a fusion protein, thereby reducing an incidence of hypoglycemia in a neonate with neonatal HI.

In another embodiment, invention provides a method of treating a disease, disorder or condition, comprising administering the pharmaceutical composition described above to a subject using multiple consecutive doses of the pharmaceutical composition administered using a therapeutically effective dose regimen. In one embodiment of the foregoing, the therapeutically effective dose regimen can result in a gain in time of at least three-fold, or alternatively, at least four-fold, or five-fold, or six-fold, or seven-fold, or eight-fold, or nine-fold, or at least 10-fold, or at least 20-fold, or at least about 30-fold, or at least about 50-fold, or at least about 100-fold longer time between at least two consecutive Cmax peaks and/or Cmin troughs for blood levels of the fusion protein compared to the corresponding AGP of the fusion protein not linked to the fusion protein and administered at a comparable dose regimen to a subject. In another embodiment of the foregoing, the administration of the fusion protein results in improvement in at least one measured parameter of a AGP-related disease using less frequent dosing or a lower total dosage in moles of the fusion protein of the pharmaceutical composition compared to the corresponding biologically active protein component(s) not linked to the fusion protein and administered to a subject using a therapeutically effective regimen to a subject.

The invention further provides use of the compositions comprising the fusion protein of any of the foregoing embodiments in the preparation of a medicament for treating a disease, disorder or condition in a subject in need thereof. In one embodiment of the foregoing, the disease, disorder or condition is selected from, but not limited to, neonatal hyperinsulinism, congential hyperinsulinism, acute hypoglycemia, nocturnal hypoglycemia, chronic hypoglycemia, Beckwith-Wiedemann syndrome, congenital disorders of glycosylation, hypoglycemia resulting from dialysis, glucagonomas, secretory disorders of the airway, arthritis, neuroendocrine tumors, osteoporosis, central nervous system disease, restenosis, neurodegenerative disease, renal failure, congestive heart failure, nephrotic syndrome, cirrhosis, pulmonary edema, hypertension, and disorders wherein the reduction of food intake is desired, stroke, irritable bowel syndrome, myocardial infarction (e.g., reducing the morbidity and/or mortality associated therewith), stroke, acute coronary syndrome (e.g., characterized by an absence of Q-wave) myocardial infarction, post-surgical catabolic changes, hibernating myocardium or diabetic cardiomyopathy, post-prandial hypoglycemia, insufficient urinary sodium excretion, excessive urinary potassium concentration, conditions or disorders associated with toxic hypervolemia, (e.g., renal failure, congestive heart failure, nephrotic syndrome, cirrhosis, pulmonary edema, and hypertension), polycystic ovary syndrome, respiratory distress, nephropathy, left ventricular systolic dysfunction, (e.g., with abnormal left ventricular ejection fraction), gastrointestinal disorders such as diarrhea, postoperative dumping syndrome and irritable bowel syndrome, (i.e., via inhibition of antro-duodenal motility), critical illness polyneuropathy (CIPN), dyslipidemia, organ tissue injury caused by reperfusion of blood flow following ischemia, and coronary heart disease risk factor (CHDRF) syndrome, and any other indication for which AGP can be utilized. Any of the disclosed embodiments can be practiced alone or in combination depending on the interested application.

The neonatal hyperinsulinism (HI) treated or ameliorated by methods of the present invention, is, in another embodiment, non-genetic HI. In another embodiment, the neonatal HI is prolonged neonatal HI. In another embodiment, the neonatal HI is non-genetic, prolonged neonatal HI. In another embodiment, the neonatal HI lasts for several months after birth. In another embodiment, the neonatal HI is the result of peri-natal stress. In another embodiment, the peri-natal stress is the result of small-for-gestational-age birth weight. In another embodiment, the peri-natal stress is the result of birth asphyxia. In another embodiment, the peri-natal stress is the result of any other peri-natal stress known in the art. Each possibility represents a separate embodiment of the present invention.

### DETAILED DESCRIPTION

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein, can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

As used herein, the following terms have the meanings ascribed to them unless specified otherwise.

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non amino acids. The terms also encompass an amino acid polymer that has been modified, for example, by disulfide bond formation, glycosylation, lipidation, acetylation, phosphorylation, or any other manipulation, such as conjugation with a labeling component.

As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine (S), and threonine (T).

The term “non-naturally occurring,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide may share no more than 99%, 98%, 95%, 90%, 80%, 70%, 60%, 50% or even less amino acid sequence identity as compared to a natural sequence when suitably aligned.

The terms “hydrophilic” and “hydrophobic” refer to the degree of affinity that a substance has with water. A hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be characterized based on their hydrophobicity. A number of scales have been developed. An example is a scale developed by Levitt, M, et al., J Mol Biol (1976) 104:59, which is listed in Hopp, T P, et al., Proc Natl Acad Sci USA (1981) 78:3824. Examples of “hydrophilic amino acids” are arginine, lysine, threonine, alanine, asparagine, and glutamine. Of particular interest are the hydrophilic amino acids aspartate, glutamate, and serine, and glycine. Examples of “hydrophobic amino acids” are tryptophan, tyrosine, phenylalanine, methionine, leucine, isoleucine, and valine.

A “fragment” is a truncated form of a native biologically active protein that retains at least a portion of the therapeutic and/or biological activity. A “variant” is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity with the reference biologically active protein.

As used herein, the term “biologically active protein moiety” includes proteins modified deliberately, as for example, by site directed mutagenesis, insertions, or accidentally through mutations.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

A “chimeric” protein contains at least one fusion polypeptide comprising regions in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Conjugated”, “linked,” “fused,” and “fusion” are used interchangeably herein. These terms refer to the joining together of two or more chemical elements or components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two or more segments that correspond to polypeptides encoded by the original ORFs (which segments are not normally so joined in nature).

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” or “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

“Homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, or at least 80%, or at least 90%, or 95%, or 97%, or 98%, or 99% sequence identity to those sequences.

“Ligation” refers to the process of forming phosphodiester bonds between two nucleic acid fragments or genes, linking them together. To ligate the DNA fragments or genes together, the ends of the DNA must be compatible with each other. In some cases, the ends will be directly compatible after endonuclease digestion. However, it may be necessary to first convert the staggered ends commonly produced after endonuclease digestion to blunt ends to make them compatible for ligation.

The terms “stringent conditions” or “stringent hybridization conditions” includes reference to conditions under which a polynucleotide will hybridize to its target sequence, to a detectably greater degree than other sequences (e.g., at least 2-fold over background). Generally, stringency of hybridization is expressed, in part, with reference to the temperature and salt concentration under which the wash step is carried out. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes for 15 min each in 0.1×SSC/1% SDS at 60 to 65° C. Alternatively, temperatures of about 65° C., 60° C., 55° C., or 42° C. may be used. SSC concentration may be varied from about 0.1 to 2×SSC, with SDS being present at about 0.1%. Such wash temperatures are typically selected to be about 5° C. to 20° C. lower than the thermal melting point for the specific sequence at a defined ionic strength and pH. The Tm is the temperature (under defined ionic strength and pH) at which 50% of the target sequence hybridizes to a perfectly matched probe. An equation for calculating Tm and conditions for nucleic acid hybridization are well known and can be found in Sambrook, J. et al. (1989) Molecular Cloning: A Laboratory Manual, 2nd ed., vol. 1-3, Cold Spring Harbor Press, Plainview N.Y.; specifically see volume 2 and chapter 9. Typically, blocking reagents are used to block non-specific hybridization. Such blocking reagents include, for instance, sheared and denatured salmon sperm DNA at about 100-200 pg/mL. Organic solvent, such as formamide at a concentration of about 35-50% v/v, may also be used under particular circumstances, such as for RNA:DNA hybridizations. Useful variations on these wash conditions will be readily apparent to those of ordinary skill in the art.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, for example, as defined by a particular SEQ ID number, or may be measured over a shorter length; for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) amino acid sequence identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The term “non-repetitiveness” as used herein in the context of a polypeptide refers to a lack or limited degree of internal homology in a peptide or polypeptide sequence. The term “substantially non-repetitive” can mean, for example, that there are few or no instances of four contiguous amino acids in the sequence that are identical amino acid types or that the polypeptide has a subsequence score (defined infra) of 10 or less or that there isn't a pattern in the order, from N- to C-terminus, of the sequence motifs that constitute the polypeptide sequence. The term “repetitiveness” as used herein in the context of a polypeptide refers to the degree of internal homology in a peptide or polypeptide sequence. In contrast, a “repetitive” sequence may contain multiple identical copies of short amino acid sequences. For instance, a polypeptide sequence of interest may be divided into n-mer sequences and the number of identical sequences can be counted. Highly repetitive sequences contain a large fraction of identical sequences while non-repetitive sequences contain few identical sequences. In the context of a polypeptide, a sequence can contain multiple copies of shorter sequences of defined or variable length, or motifs, in which the motifs themselves have non-repetitive sequences, rendering the full-length polypeptide substantially non-repetitive. The length of polypeptide within which the non-repetitiveness is measured can vary from 3 amino acids to about 200 amino acids, about from 6 to about 50 amino acids, or from about 9 to about 14 amino acids. “Repetitiveness” used in the context of polynucleotide sequences refers to the degree of internal homology in the sequence such as, for example, the frequency of identical nucleotide sequences of a given length. Repetitiveness can, for example, be measured by analyzing the frequency of identical sequences.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

“Serum degradation resistance,” as applied to a polypeptide, refers to the ability of the polypeptides to withstand degradation in blood or components thereof, which typically involves proteases in the serum or plasma. The serum degradation resistance can be measured by combining the protein with human (or mouse, rat, monkey, as appropriate) serum or plasma, typically for a range of days (e.g. 0.25, 0.5, 1, 2, 4, 8, 16 days), typically at about 37° C. The samples for these time points can be run on a Western blot assay and the protein is detected with an antibody. The antibody can be to a tag in the protein. If the protein shows a single band on the western, where the protein's size is identical to that of the injected protein, then no degradation has occurred. In this exemplary method, the time point where 50% of the protein is degraded, as judged by Western blots or equivalent techniques, is the serum degradation half-life or “serum half-life” of the protein.

The term “t_{1/2}” as used herein means the terminal half-life calculated as ln(2)/Kₑₗ. Kₑₗ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. The terms “t_{1/2}”, “terminal half-life”, “elimination half-life” and “circulating half-life” are used interchangeably herein.

“Apparent Molecular Weight Factor” or “Apparent Molecular Weight” are related terms referring to a measure of the relative increase or decrease in apparent molecular weight exhibited by a particular amino acid sequence. The Apparent Molecular Weight is determined using size exclusion chromatography (SEC) and similar methods compared to globular protein standards and is measured in “apparent kD” units. The Apparent Molecular Weight Factor is the ratio between the Apparent Molecular Weight and the actual molecular weight; the latter predicted by adding, based on amino acid composition, the calculated molecular weight of each type of amino acid in the composition.

The “hydrodynamic radius” or “Stokes radius” is the effective radius (Rₕ, in nm) of a molecule in a solution measured by assuming that it is a body moving through the solution and resisted by the solution's viscosity. In the embodiments of the invention, the hydrodynamic radius measurements of the FPP fusion proteins correlate with the ‘Apparent Molecular Weight Factor, which is a more intuitive measure. The “hydrodynamic radius” of’ a protein affects its rate of diffusion in aqueous solution as well as its ability to migrate in gels of macromolecules. The hydrodynamic radius of a protein is determined by its molecular weight as well as by its structure, including shape and compactness. Methods for determining the hydrodynamic radius are well known in the art, such as by the use of size exclusion chromatography (SEC), as described in U.S. Pat. Nos. 6,406,632 and 7,294,513. Most proteins have globular structure, which is the most compact three-dimensional structure a protein can have with the smallest hydrodynamic radius. Some proteins adopt a random and open, unstructured, or ‘linear’ conformation and as a result have a much larger hydrodynamic radius compared to typical globular proteins of similar molecular weight.

“Physiological conditions” refer to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers are listed in Sambrook et al. (1989). Physiologically relevant temperature ranges from about 25° C. to about 38° C., and preferably from about 35° C. to about 37° C.

A “reactive group” is a chemical structure that can be coupled to a second reactive group. Examples for reactive groups are amino groups, carboxyl groups, sulfhydryl groups, hydroxyl groups, aldehyde groups, azide groups. Some reactive groups can be activated to facilitate coupling with a second reactive group. Examples for activation are the reaction of a carboxyl group with carbodiimide, the conversion of a carboxyl group into an activated ester, or the conversion of a carboxyl group into an azide function.

“Controlled release agent”, “slow release agent”, “depot formulation” or “sustained release agent” are used interchangeably to refer to an agent capable of extending the duration of release of a polypeptide of the invention relative to the duration of release when the polypeptide is administered in the absence of agent. Different embodiments of the present invention may have different release rates, resulting in different therapeutic amounts.

The terms “antigen”, “target antigen” or “immunogen” are used interchangeably herein to refer to the structure or binding determinant that an antibody fragment or an antibody fragment-based therapeutic binds to or has specificity against.

The term “payload” as used herein refers to a protein or peptide sequence that has biological or therapeutic activity; the counterpart to the pharmacophore of small molecules. Examples of payloads include, but are not limited to, cytokines, enzymes, hormones and blood and growth factors. Payloads can further comprise genetically fused or chemically conjugated moieties such as chemotherapeutic agents, antiviral compounds, toxins, or contrast agents. These conjugated moieties can be joined to the rest of the polypeptide via a linker that may be cleavable or non-cleavable.

The term “antagonist”, as used herein, includes any molecule that partially or fully blocks, inhibits, or neutralizes a biological activity of a native polypeptide disclosed herein. Methods for identifying antagonists of a polypeptide may comprise contacting a native polypeptide with a candidate antagonist molecule and measuring a detectable change in one or more biological activities normally associated with the native polypeptide. In the context of the present invention, antagonists may include proteins, nucleic acids, carbohydrates, antibodies or any other molecules that decrease the effect of a biologically active protein.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

“Activity” for the purposes herein refers to an action or effect of a component of a fusion protein consistent with that of the corresponding native biologically active protein, wherein “biological activity” refers to an in vitro or in vivo biological function or effect, including but not limited to receptor binding, antagonist activity, agonist activity, or a cellular or physiologic response.

As used herein, “treatment” or “treating,” or “palliating” or “ameliorating” is used interchangeably herein. These terms refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect”, as used herein, refers to a physiologic effect, including but not limited to the cure, mitigation, amelioration, or prevention of disease in humans or other animals, or to otherwise enhance physical or mental wellbeing of humans or animals, caused by a fusion polypeptide of the invention other than the ability to induce the production of an antibody against an antigenic epitope possessed by the biologically active protein. Determination of a therapeutically effective amount is well within the capability of those skilled in the art, especially in light of the detailed disclosure provided herein.

The terms “therapeutically effective amount” and “therapeutically effective dose”, as used herein, refers to an amount of a biologically active protein, either alone or as a part of a fusion protein composition, that is capable of having any detectable, beneficial effect on any symptom, aspect, measured parameter or characteristics of a disease state or condition when administered in one or repeated doses to a subject. Such effect need not be absolute to be beneficial.

The term “therapeutically effective dose regimen”, as used herein, refers to a schedule for consecutively administered doses of a biologically active protein, either alone or as a part of a fusion protein composition, wherein the doses are given in therapeutically effective amounts to result in sustained beneficial effect on any symptom, aspect, measured parameter or characteristics of a disease state or condition.

The peptides of the present invention include amino acid sequences with and without an added N-terminal methionine. Those of skill will understand that the addition of the methionine to the N-terminus will depend on the expression system, e.g. E. coli, used to produce the polypeptide. It is understood that the N-terminals of the exemplary peptides can start with or without methionine. In addition, those of skill will understand that the strategy for preparing the N-terminal containing peptides is applicable to any peptide.

In various embodiments, the modified AGP and AGP-FPP of the invention having an N-terminal Met has the advantage of being obtainable by recombinant means, such as by production in E. coli or other expression system, without further post-expression manufacturing processes to expose the natural or desired VIP N-terminus.

### I) General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3rd edition, Cold Spring Harbor Laboratory Press, 2001; “Current protocols in molecular biology”, F. M. Ausubel, et al. eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11th Edition, McGraw-Hill, 2005; and Freshney, R. I., “Culture of Animal Cells: A Manual of Basic Technique,” 4th edition, John Wiley & Sons, Somerset, N.J., 2000, the contents of which are incorporated in their entirety herein by reference.

The present invention relates in part to fusion protein compositions comprising antagonists of Glucagon-Like Peptide (GLP-1) receptor (AGP). Such compositions can have utility in the treatment or prevention of certain diseases, disorder or conditions related to glucose homeostasis, insulin oversecretion, dyslipidemia, hypertension, and the like.

This invention provides methods of treating and ameliorating congenital and neonatal hyperinsulinism and post-prandial hypoglycemia, comprising the step of administering an antagonist of the Glucagon-Like Peptide-1 (GLP-1) receptor fusion protein.

In one embodiment, the present invention provides a method of treating a subject with a congenital hyperinsulinism, comprising the step of administering to the subject an antagonist of the GLP-1 receptor fusion protein, thereby treating a subject with a congenital hyperinsulinism.

In another embodiment, the present invention provides a method of reducing an incidence of hypoglycemia in a subject with congenital hyperinsulinism, comprising the step of administering to the subject an antagonist of the GLP-1 receptor (GLP-1R)-fusion protein, thereby reducing an incidence incidence of hypoglycemia in a subject with congenital hyperinsulinism.

In another embodiment, the present invention provides a method of ameliorating a congenital hyperinsulinism in a subject, comprising the step of administering to the subject an antagonist peptide of the GLP-1 receptor-fusion protein (AGP-FPP), thereby ameliorating a congenital hyperinsulinism in a subject.

In another embodiment, the present invention provides a method of inhibiting a development of hypoglycemia in a subject with congenital hyperinsulinism, comprising the step of administering to the subject an antagonist of GLP-1R-fusion protein, thereby inhibiting a development of hypoglycemia in a subject with congenital hyperinsulinism.

In another embodiment, the present invention provides a method of increasing fasting blood glucose levels and improving fasting tolerance in a subject with congenital hyperinsulinism, comprising the step of administering to the subject an antagonist of GLP-1R-fusion protein, increasing fasting blood glucose levels in a subject with congenital hyperinsulinism.

In another embodiment, the present invention provides a method of decreasing the glucose requirement to maintain normoglycemia of a subject with congenital hyperinsulinism, comprising the step of administering to the subject an antagonist of GLP-1R-fusion protein, thereby decreasing the glucose requirement to maintain euglycemia of a subject with congenital hyperinsulinism.

In one embodiment, AGP-FPP desribed herein, suppresses amino acid-stimulated insulin secretion. In another embodiment AGP-FPP described herein, blocks the abnormal nutrient stimulation of insulin secretion in the absence of functional K.sup.+ATP channels. In one embodiment, AGP-FPP described herein, decreases basal and amino-acid stimulated insulin secretion and intracellular cAMP accumulation. Accordingly and in one embodiment, AGLP-FPP corrects the abnormal pattern of insulin secretion responsible for hypoglycemia: basal elevated insulin secretion in the absence of glucose and the amino acid-stimulated insulin secretion.

In another embodiment, the GLP-1 receptor antagonist-fusion protein (AGP-FPP) suppresses insulin secretion by the subject.

In another embodiment, the GLP-1 receptor antagonist-fusion protein is administered after diagnosis of congenital hyperinsulinism. In another embodiment, the GLP-1 receptor antagonist fusion protein is administered after identification of a genetic abnormality that predisposes to congenital hyperinsulinism. In another embodiment, the GLP-1 receptor antagonist fusion protein is administered to a subject with a family history of congenital hyperinsulinism. Each possibility represents a separate embodiment of the present invention.

In one embodiment, cyclic AMP stimulates exocytosis by PKA-dependent pathways, through phosphorylation of downstream targets including the K_{ATP} channel, and by PKA-independent mechanisms, through the activation of guanine nucleotide exchange factors (GEFs) such as cAMP-GEFII (also known as Epac). The PKA-independent pathway is critical in another embodiment in the potentiation of insulin secretion by the incretin hormones GLP-1 and GIP and in one embodiment, exerts its effect on insulin containing secretory granules located in the readily releasable pool. In pancreatic islets, the effect of cAMPGEFII on insulin secretion depends in one embodiment on cytosolic calcium as well as cAMP, and cAMP sensitizes in another embodiment the exocytotic machinery to calcium. In one embodiment, the inhibition of insulin secretion in SUR-1_{-/-} islets by AGP-FPP described herein, is mediated by the effect of cAMP on a late calcium-dependent step in the exocytotic pathway involving the readily releasable pool of insulin granules.

The congenital hyperinsulinism treated or ameliorated by methods of the present invention, is, in another embodiment, associated with increases insulin secretion by the subject. In another embodiment, the congenital hyperinsulinism is associated with a genetic abnormality. In another embodiment, the congenital hyperinsulinism is associated with a genetic mutation. In another embodiment, the congenital hyperinsulinism is a result of a genetic abnormality. In another embodiment, the congenital hyperinsulinism is a result of a genetic mutation. Each possibility represents another embodiment of the present invention.

In another embodiment, the congenital hyperinsulinism is associated with a K_{ATP} channel dysfunction. In another embodiment, the congenital hyperinsulinism is a K_{ATP} hyperinsulinism.

In another embodiment, the congenital hyperinsulinism is associated with a mutation in a gene encoding a sulfonylurea receptor (ABCC8). In another embodiment, the congenital hyperinsulinism is associated with a mutation in a gene encoding an inward rectifying potassium channel, Kir6.2 protein (KCNJ11). In another embodiment, the congenital hyperinsulinism is associated with a mutation in a gene encoding a glucokinase (GCK). In another embodiment, the congenital hyperinsulinism is associated with a mutation in a gene encoding a glutamate dehydrogenase (GLUD-1). In another embodiment, the congenital hyperinsulinism is associated with a mutation in a gene encoding a mitochondrial enzyme short-chain 3-hydroxyacyl-CoA dehydrogenase (HADHSC). In another embodiment, the congenital hyperinsulinism is associated with any other mutation known in the art to be associated with a congenital hyperinsulinism. Each possibility represents another embodiment of the present invention.

In another embodiment, the present invention provides a method of treating a subject with a post-prandial hypoglycemia, comprising the step of administering to the subject an antagonist of the GLP-1 receptor fusion protein, thereby treating a subject with a post-prandial hypoglycemia. In another embodiment, the post-prandial hypoglycemia is associated with gastric-bypass surgery.

In another embodiment, the present invention provides a method of reducing an incidence of a post-prandial hypoglycemia in a subject, comprising the step of administering to the subject an antagonist of GLP-1R-fusion protein, thereby reducing an incidence of a post-prandial hypoglycemia in a subject.

In another embodiment, the present invention provides a method of ameliorating a post-prandial hypoglycemia in a subject, comprising the step of administering to the subject an antagonist of GLP-1R-fusion protein, thereby ameliorating a post-prandial hypoglycemia in a subject.

In another embodiment, the present invention provides a method of inhibiting a development of a post-prandial hypoglycemia in a subject, comprising the step of administering to the subject an antagonist of GLP-1R-fusion protein, thereby inhibiting a development of a post-prandial hypoglycemia in a subject.

In another embodiment, the present invention provides a method of decreasing the glucose requirement to maintain euglycemia of a subject with post-prandial hypoglycemia, comprising the step of administering to the subject an antagonist of GLP-1R-fusion protein, thereby decreasing the glucose requirement to maintain euglycemia of a subject with post-prandial hypoglycemia.

In another embodiment, the GLP-1R antagonist-fusion protein suppresses insulin secretion by the subject.

The post-prandial hypoglycemia treated or inhibited by methods and compositions of the present invention is, in another embodiment, associated with a Nissen fundoplication. In another embodiment, the post-prandial hypoglycemia occurs following a Nissen fundoplication. Each possibility represents a separate embodiment of the present invention.

In another embodiment, the post-prandial hypoglycemia is associated with a gastric-bypass surgery. In another embodiment, the post-prandial hypoglycemia occurs following a gastric-bypass surgery. Each possibility represents a separate embodiment of the present invention.

In another embodiment, the antagonist peptide of GLP-1 receptor (AGP)-fusion protein is administered after diagnosis of post-prandial hypoglycemia.

In another embodiment, the GLP-1R antagonist-fusion protein is administered after a gastric-bypass surgery. In another embodiment, the GLP-1R antagonist-fusion protein is administered during a gastric-bypass surgery. In another embodiment, the GLP-1R antagonist-fusion protein is administered prior to a gastric-bypass surgery.

In another embodiment, the AGP-FPP is administered after a Nissen fundoplication. In another embodiment, the AGP-FPP is administered during a Nissen fundoplication. In another embodiment, the AGP-FPP is administered prior to a Nissen fundoplication.

In another embodiment, the present invention provides a method of treating a subject with a neonatal HI, comprising the step of administering to the subject AGP-FPP, thereby treating a subject with a neonatal HI.

In another embodiment, the present invention provides a method of reducing an incidence of hypoglycemia in a subject with neonatal HI, comprising the step of administering to the subject AGP-FPP, thereby reducing an incidence of hypoglycemia in a subject with neonatal HI.

The neonatal hyperinsulinism (HI) treated or ameliorated by methods of the present invention, is, in another embodiment, non-genetic HI. In another embodiment, the neonatal HI is prolonged neonatal HI. In another embodiment, the neonatal HI is non-genetic, prolonged neonatal HI. In another embodiment, the neonatal HI lasts for several months after birth. In another embodiment, the neonatal HI is the result of peri-natal stress. In another embodiment, the peri-natal stress is the result of small-for-gestational-age birth weight. In another embodiment, the peri-natal stress is the result of birth asphyxia. In another embodiment, the peri-natal stress is the result of any other peri-natal stress known in the art. Each possibility represents a separate embodiment of the present invention.

The AGP of the AGP-FPP utilized in methods and compositions of the present invention is, in another embodiment, is a GLP-1 analogue. In another embodiment, the analogue is an antagonist of a GLP-1 receptor. Each possibility represents a separate embodiment of the present invention.

In another embodiment, the present invention provides a method of ameliorating the hypoglycemia in a Beckwith-Wiedemann syndrome subject, comprising the step of administering to the subject an antagonist peptide of the GLP-1 receptor-fusion protein (AGP-FPP), thereby ameliorating the hypoglycemia in the subject.

The hyperinsulinism of a Beckwith-Wiedemann syndrome subject treated or ameliorated by methods of the present invention, is, in another embodiment, associated with increases insulin secretion by the subject. In another embodiment, the hyperinsulinemia in a Beckwith-Wiedemann syndrome subject is associated with a genetic abnormality. In another embodiment, the hyperinsulinemia in a Beckwith-Wiedemann syndrome subject is associated with a genetic mutation. Each possibility represents another embodiment of the present invention.

In another embodiment, the present invention provides a method of ameliorating the hypoglycemia in a subject with congenital glycosylation disorder, comprising the step of administering to the subject an antagonist peptide of the GLP-1 receptor-fusion protein (AGP-FPP), thereby ameliorating the hypoglycemia in the subject.

The hyperinsulinism of a congenital glycosylation disorder treated or ameliorated by methods of the present invention, is, in another embodiment, associated with increases insulin secretion by the subject. In another embodiment, the hyperinsulinemia in a congenital glycosylation disorder is associated with a genetic abnormality. In another embodiment, the hyperinsulinemia in a congenital glycosylation disorder is associated with a genetic mutation. Each possibility represents another embodiment of the present invention.

The hyperinsulinism of a Beckwith-Wiedemann syndrome subject treated or ameliorated by methods of the present invention, is, in another embodiment, associated with increases insulin secretion by the subject. In another embodiment, the hyperinsulinemia in a Beckwith-Wiedemann syndrome subject is associated with a genetic abnormality. In another embodiment, the hyperinsulinemia in a Beckwith-Wiedemann syndrome subject is associated with a genetic mutation. Each possibility represents another embodiment of the present invention.

In another embodiment, the present invention provides a method of ameliorating the hypoglycemia in a subject with kidney disease, comprising the step of administering to the subject an antagonist peptide of the GLP-1 receptor-fusion protein (AGP-FPP), thereby ameliorating the hypoglycemia in the subject. In another embodiment, the kidney disease subject is undergoing dialysis. In another embodiment, the hypoglycemia is associated with dialysis. Each possibility represents another embodiment of the present invention.

The hyperinsulinism of kidney disease is treated or ameliorated by methods of the present invention, is, in another embodiment, associated with increases insulin secretion by the subject. In another embodiment, the hyperinsulinemia in kidney disease is associated with a genetic abnormality. In another embodiment, the hyperinsulinemia in kidney disease is associated with a genetic mutation. In another embodiment, the hyperinsulinemia in kidney disease is associated with dialysis. Each possibility represents another embodiment of the present invention.

In another embodiment, the analogue is resistant to cleavage by dipeptidyl peptidase-IV (DPPIV). In another embodiment, the analogue exhibits an extended biological half-life relative to GLP-1. In another embodiment, the analogue is resistant to degradation by DPPIV. Each possibility represents another embodiment of the present invention.

“Resistant to cleavage” refers, in another embodiment, to resistance to proteolysis by DPPIV relative to GLP-1. In another embodiment, the term refers to resistance relative to a GLP-1 fragment. In another embodiment, the term refers to resistance to proteolysis by another dipeptidyl peptidase. In another embodiment, the dipeptidyl peptidase is DPP10 (dipeptidyl peptidase IV-related protein 3). In another embodiment, the dipeptidyl peptidase is DPP7. In another embodiment, the dipeptidyl peptidase is DPP6. In another embodiment, the dipeptidyl peptidase is DPP3. In another embodiment, the dipeptidyl peptidase is DPP9. In another embodiment, the dipeptidyl peptidase is any other dipeptidyl peptidase known in the art. In another embodiment, the term refers to resistance to proteolysis by any other protease known in the art. In another embodiment, the term refers to any other definition of “protease resistant” known in the art. Each possibility represents a separate embodiment of the present invention.

In another embodiment, the GLP-1R antagonist utilized in methods and compositions of the present invention exhibits an improvement in a desirable biological property relative to AGP. In another embodiment, the biological property is improved biological half-life. In another embodiment, the biological property is improved affinity for GLP-1 receptor. In another embodiment, the biological property is improved potency for antagonism of GLP-1 receptor. In another embodiment, the biological property is any other desirable biological property known in the art. Each possibility represents a separate embodiment of the present invention.

In another embodiment, the antagonists are selected from Seq ID No. 19, 20 and 21. In another embodiment, the AGP-FPP contains AGP as a fragment of the peptide set forth in SEQ ID No. 1. In another embodiment, the fragment is an antagonist of a GLP-1R. In another embodiment, the fragment exhibits an extended biological half-life relative to GLP-1. In another embodiment, the fragment is resistant to cleavage by DPPIV. In another embodiment, the fragment is resistant to degradation by DPPIV. Each possibility represents another embodiment of the present invention.

In another embodiment, the AGP-FPP is Seq ID No. 19. In another embodiment, the AGP peptide has the sequence: DLSKQMEEEAVRLFIEWLKNGGPSSGAPPPS (SEQ ID No: 1). In another embodiment, the AGP is a homologue of SEQ ID No: 1. In another embodiment, the AGP is an analogue of SEQ ID No: 1. In another embodiment, the AGP is a variant of SEQ ID No: 1. In another embodiment, the AGP is any other AGP peptide known in the art. Each possibility represents a separate embodiment of the present invention.

In another embodiment, the antagonist fragment of AGP-FPP has the sequence:

(SEQ ID No: 13)
MKIILWLCVFGLFLATLFPVSWQMPVESGLSSEDSASSESFASKIKRHSD
GTFTSDLSKQMEEEAVRLFIEWLKNGGPSSGAPPPSG.

In another embodiment, the AGP peptide of AGP-FPP is a homologue of SEQ ID No: 13. In another embodiment, the AGP peptide of AGP-FPP is an analogue of SEQ ID No: 13. In another embodiment, the AGP peptide of AGP-FPP is a variant of SEQ ID No: 13. In another embodiment, the AGP peptide of AGP-FPP is any other exendin protein known in the art. Each possibility represents a separate embodiment of the present invention.

In another embodiment, the AGP peptide of AGP-FPP has the sequence:

(SEQ ID No: 2)
HAEGTFTSDVSSYLEGQAAKEFIAWLVKGR.

In another embodiment, the AGP peptide of AGP-FPP is a homologue of SEQ ID No: 2. In another embodiment, the AGP peptide of AGP-FPP is an analogue of SEQ ID No: 2. In another embodiment, the AGP peptide of AGP-FPP is a variant of SEQ ID No: 2. In another embodiment, the AGP peptide of AGP-FPP is any other GLP-1 (9-36) known in the art. Each possibility represents a separate embodiment of the present invention.

In another embodiment, the AGP peptide of AGP-FPP is a GLP-1 (7-36) containing a mutation. In another embodiment, the mutation confers GLP-1 receptor (GLP-1R) antagonistic activity. In another embodiment, the mutation reduces or eliminates GLP-1R agonistic activity. In another embodiment, the mutation does not reduce binding to GLP-1R. In another embodiment, the mutation is a substitution. In another embodiment, the mutation is an insertion. In another embodiment, the mutation is a deletion. In another embodiment, the mutation is a Glu9Lys mutation. In another embodiment, the mutation is any other type of mutation known in the art. Each possibility represents a separate embodiment of the present invention.

In another embodiment, the AGP peptide of AGP-FPP has the sequence HGEGTFTSDLSKQMEEEAVRLFIEWLKNGGPSSGAPPPS (SEQ ID No: 3). In another embodiment, the AGP peptide of AGP-FPP is a homologue of SEQ ID No: 3. In another embodiment, the AGP peptide of AGP-FPP is an analogue of SEQ ID No: 3. In another embodiment, the AGP peptide of AGP-FPP is a variant of SEQ ID No: 3. In another embodiment, the AGP peptide of AGP-FPP is any other exendin (1-39) known in the art. Each possibility represents a separate embodiment of the present invention.

In another embodiment, the sequence of the AGP peptide of AGP-FPP is:

(SEQ ID No: 8)
HAEGTFTSKVSSYLEGQAAKEFIAWLVKGR.

In another embodiment, the AGP peptide of AGP-FPP is a homologue of SEQ ID No: 8. In another embodiment, the AGP peptide of AGP-FPP is an analogue of SEQ ID No: 8. In another embodiment, the AGP peptide of AGP-FPP is a variant of SEQ ID No: 8. In another embodiment, the AGP peptide of AGP-FPP is any other mutated GLP-1 (7-36) known in the art. Each possibility represents a separate embodiment of the present invention.

In another embodiment, the AGP peptide of AGP-FPP is:

(Seq ID No. 18)
GEGTFTSELSKQMEEEAVRLFIEWLKNGGPSSGAPPPS.

In another embodiment, the AGP peptide of AGP-FPP is a homologue of SEQ ID No: 18. In another embodiment, the AGP peptide of AGP-FPP is an analogue of SEQ ID No: 18. In another embodiment, the AGP peptide of AGP-FPP is a variant of SEQ ID No: 18. In another embodiment, the AGP peptide of AGP-FPP is any other mutated GLP-1 (7-36) known in the art. Each possibility represents a separate embodiment of the present invention.

In another embodiment, the AGP peptide of AGP-FPP has the sequence:

(SEQ ID No: 4)
GEGTFTWELSKQMEEEAVRLFIEWLKNGGPSSGAPPPS.

In another embodiment, the AGP peptide of AGP-FPP is a homologue of SEQ ID No: 4. In another embodiment, the AGP peptide of AGP-FPP is an analogue of SEQ ID No: 4. In another embodiment, the AGP peptide of AGP-FPP is a variant of SEQ ID No: 4. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 5)
GEGTFTSQLSKQMEEEAVRLFIEWLKNGGPSSGAPPPS.

In another embodiment, the AGP peptide of AGP-FPP is a homologue of SEQ ID No: 5. In another embodiment, the AGP peptide of AGP-FPP is an analogue of SEQ ID No: 5. In another embodiment, the AGP peptide of AGP-FPP is a variant of SEQ ID No: 5. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 6)
KRHSDGTFTSDLSKQMEEEAVRLFIEWLKNGGPSSGAPPPS.

In another embodiment, the AGP peptide of AGP-FPP is a homologue of SEQ ID No: 6. In another embodiment, the AGP peptide of AGP-FPP is an analogue of SEQ ID No: 6. In another embodiment, the AGP peptide of AGP-FPP is a variant of SEQ ID No: 6. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 7)
HSDGTFSDLSKGMEEEAVRLHEWLKNGGPSSGAPPPS.

In another embodiment, the AGP peptide of AGP-FPP is a homologue of SEQ ID No: 7. In another embodiment, the AGP peptide of AGP-FPP is an analogue of SEQ ID No: 7. In another embodiment, the AGP peptide of AGP-FPP is a variant of SEQ ID No: 7. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 9)
HAEGTFTSKVSSYLEGQAAKEFIAWLVKGR.

In another embodiment, the AGP peptide of AGP-FPP is a homologue of SEQ ID No: 9. In another embodiment, the AGP peptide of AGP-FPP is an analogue of SEQ ID No: 9. In another embodiment, the AGP peptide of AGP-FPP is a variant of SEQ ID No: 9. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 10)
GEGTFTSDLSKQMEEEAVRLFIEWLKNGGPSSGAPPPS.

In another embodiment, the AGP peptide of AGP-FPP is a homologue of SEQ ID No: 10. In another embodiment, the AGP peptide of AGP-FPP is an analogue of SEQ ID No: 10. In another embodiment, the AGP peptide of AGP-FPP is a variant of SEQ ID No: 10. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 11)
EGTFTSDLSKQMEEEAVRLFIEWLKNGGPSSGAPPPS.

In another embodiment, the AGP peptide of AGP-FPP is a homologue of SEQ ID No: 11. In another embodiment, the AGP peptide of AGP-FPP is an analogue of SEQ ID No: 11. In another embodiment, the AGP peptide of AGP-FPP is a variant of SEQ ID No: 11. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 12)
GTFTSDLSKQMEEEAVRLFIEWLKNGGPSSGAPPPS.

In another embodiment, the AGP peptide of AGP-FPP is a homologue of SEQ ID No: 12. In another embodiment, the AGP peptide of AGP-FPP is an analogue of SEQ ID No: 12. In another embodiment, the AGP peptide of AGP-FPP is a variant of SEQ ID No: 12. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 14)
SDLSKQMEEEAVRLFIEWLKNGGPSSGAPPPS.

In another embodiment, the AGP peptide of AGP-FPP is a homologue of SEQ ID No: 14. In another embodiment, the AGP peptide of AGP-FPP is an analogue of SEQ ID No: 14. In another embodiment, the AGP peptide of AGP-FPP is a variant of SEQ ID No: 14. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 15)
TFTSDLSKQMEEEAVRLFIEWLKNGGPSSGAPPPS.

In another embodiment, the AGP peptide of AGP-FPP is a homologue of SEQ ID No: 15. In another embodiment, the AGP peptide of AGP-FPP is an analogue of SEQ ID No: 15. In another embodiment, the AGP peptide of AGP-FPP is a variant of SEQ ID No: 15. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 16)
FTSDLSKQMEEEAVRLFIEWLKNGGPSSGAPPPS.

In another embodiment, the AGP peptide of AGP-FPP is a homologue of SEQ ID No: 16. In another embodiment, the AGP peptide of AGP-FPP is an analogue of SEQ ID No: 16. In another embodiment, the AGP peptide of AGP-FPP is a variant of SEQ ID No: 16. In another embodiment, the AGP peptide of AGP-FPP is any other mutated GLP-1 (7-36) known in the art. Each possibility represents a separate embodiment of the present invention.

In another embodiment, the sequence of the AGP peptide of AGP-FPP is:

(Seq ID No. 17)
TSDLSKQMEEEAVRLFIEWLKNGGPSSGAPPPS.

In another embodiment, the AGP peptide of AGP-FPP is a homologue of SEQ ID No: 17. In another embodiment, the AGP peptide of AGP-FPP is an analogue of SEQ ID No: 17. In another embodiment, the AGP peptide of AGP-FPP is a variant of SEQ ID No: 17. In another embodiment, the AGP peptide of AGP-FPP is any other mutated GLP-1 (7-36) known in the art. Each possibility represents a separate embodiment of the present invention.

In another embodiment, the sequence of the FPP peptide of AGP-FPP is:

(Seq ID No. 56)
VPDKTVRWCAVSEHEATKCQSFRDHMKSVIPSDGPSVACVKKASYLDCIR
AIAANEADAVTLDAGLVYDAYLAPNNLKPVVAEFYGSKEDPQTFYYAVAV
VKKDSGFQMNQLRGKKSCHTGLGRSAGWNIPIGLLYCDLPEPRKPLEKAV
ANFFSGSCAPCADGTDFPQLCQLCPGCGCSTLNQYFGYSGAFKCLKDGAG
DVAFVKHSTIFENLANKADRDQYELLCLDNTRKPVDEYKDCHLAQVPSHT
VVARSMGGKEDLIWELLNQAQEHFGKDKSKEFQLFSSPHGKDLLFKDSAH
GFLKVPPRMDAKMYLGYEYVTAIRNLREGTCPEAPTDECKPVKWCALSHH
ERLKCDEWSVNSVGKIECVSAETTEDCIAKIMNGEADAMSLDGGFVYIAG
KCGLVPVLAENYNKSDNCEDTPEAGYFAIAVVKKSASDLTWDNLKGKKSC
HTAVGRTAGWNIPMGLLYNKINHCRFDEFFSEGCAPGSKKDSSLCKLCMG
SGLNLCEPNNKEGYYGYTGAFRCLVEKGDVAFVKHQTVPQNTGGKNPDPW
AKNLNEKDYELLCLDGTRKPVEEYANCHLARAPNHAVVTRKDKEACVHKI
LRQQQHLFGSNVTDCSGNFCLFRSETKDLLFRDDTVCLAKLHDRNTYEKY
LGEEYVKAVGNLRKCSTSSLLEACTFRRP.

In another embodiment, the FPP peptide of AGP-FPP is a homologue of SEQ ID No: 56. In another embodiment, the FPP peptide of AGP-FPP is an analogue of SEQ ID No: 56. In another embodiment, the FPP peptide of AGP-FPP is a variant of SEQ ID No: 56. In another embodiment, the FPP peptide of AGP-FPP is any other mutated transferrin known in the art. Each possibility represents a separate embodiment of the present invention.

In another embodiment, the sequence of the FPP peptide of AGP-FPP is:

(Seq ID No. 57)
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL.

In another embodiment, the FPP peptide of AGP-FPP is a homologue of SEQ ID No: 57. In another embodiment, the FPP peptide of AGP-FPP is an analogue of SEQ ID No: 57. In another embodiment, the FPP peptide of AGP-FPP is a variant of SEQ ID No: 57. In another embodiment, the FPP peptide of AGP-FPP is any other mutated albumin known in the art. Each possibility represents a separate embodiment of the present invention.

In another embodiment, the sequence of the FPP peptide of AGP-FPP is:

(Seq ID No. 58)
GSPAGSPTSTEEGTSESATPESGPGTSTEPSEGSAPGSPAGSPTSTEEGT
STEPSEGSAPGTSTEPSEGSAPGTSESATPESGPGSEPATSGSETPGSEP
ATSGSETPGSPAGSPTSTEEGTSESATPESGPGTSTEPSEGSAPGTSTEP
SEGSAPGSPAGSPTSTEEGTSTEPSEGSAPGTSTEPSEGSAPGTSESATP
ESGPGTSTEPSEGSAPGTSESATPESGPGSEPATSGSETPGTSTEPSEGS
APGTSTEPSEGSAPGTSESATPESGPGTSESATPESGPGSPAGSPTSTEE
GTSESATPESGPGSEPATSGSETPGTSESATPESGPGTSTEPSEGSAPGT
STEPSEGSAPGTSTEPSEGSAPGTSTEPSEGSAPGTSTEPSEGSAPGTST
EPSEGSAPGSPAGSPTSTEEGTSTEPSEGSAPGTSESATPESGPGSEPAT
SGSETPGTSESATPESGPGSEPATSGSETPGTSESATPESGPGTSTEPSE
GSAPGTSESATPESGPGSPAGSPTSTEEGSPAGSPTSTEEGSPAGSPTST
EEGTSESATPESGPGTSTEPSEGSAPGTSESATPESGPGSEPATSGSETP
GTSESATPESGPGSEPATSGSETPGTSESATPESGPGTSTEPSEGSAPGS
PAGSPTSTEEGTSESATPESGPGSEPATSGSETPGTSESATPESGPGSPA
GSPTSTEEGSPAGSPTSTEEGTSTEPSEGSAPGTSESATPESGPGTSESA
TPESGPGTSESATPESGPGSEPATSGSETPGSEPATSGSETPGSPAGSPT
STEEGTSTEPSEGSAP.

In another embodiment, the FPP peptide of AGP-FPP is a homologue of SEQ ID No: 58. In another embodiment, the FPP peptide of AGP-FPP is an analogue of SEQ ID No: 58. In another embodiment, the FPP peptide of AGP-FPP is a variant of SEQ ID No: 58. In another embodiment, the FPP peptide of AGP-FPP is any other XTEN peptide described in US Pat. Appl No. 20100323956 and are incorporated herein. In another embodiment, the FPP peptide is an Elastin-Like-Peptide (ELP), as described in US Pat. Appl. No. 20110178017 and US Pat. Appl. No. 200803240 and incorporated by reference, and is fused to an N- or C-terminal of the AGP peptide. Each possibility represents a separate embodiment of the present invention.

In some embodiments, the FPP of the invention is a bioelastic polymer (ELP) component fused to an N-terminal and/or C-terminal AGP peptide. A “bioelastic polymer” may exhibit an inverse temperature transition. Bioelastic polymers are known and described in, for example, U.S. Pat. No. 5,520,672 to Urry et al., Bioelastic polymers may be polypeptides comprising elastomeric units of pentapeptides, tetrapeptides, and/or nonapeptides (e.g. “elastin-like peptides”). Bioelastic polymers that may be used to carry out the present invention are net forth in U.S. Pat. No. 4,474,851, which describes a number of tetrapeptide and pentapeptide repeating units that can be used to form a bioelastic polymer. Specific bioelastic polymers are also described in U.S. Pat. Nos. 4,132,746; 4,187,852; 4,500,700; 4,589,882; and 4,870,055. Still other examples of bioelastic polymers are set forth in U.S. Pat. No. 6,699,294, U.S. Pat. No. 6,753,311, and U.S. Pat. No. 6,063,061. The structures of such bioelastic polymers are hereby incorporated by reference.

In one embodiment, the bioelastic polymers are polypeptides of the general formula (VPGXG)ₘ where X is any amino acid (e.g., Ala, Leu, Phe) and m is from about 20 to about 2000, or about 50 to about 180. In exemplary embodiments, m is 60, 90, 120, 150, or 180. The frequency of the various amino acids as the fourth amino acid can be changed, as well as the identity of X.

In another embodiment, the sequence of the FPP peptide of AGP-FPP is:

(Seq ID No. 59)
GTSTEPSEGSAPGSEPATSGSETPGSPAGSPTSTEEGSTSSTAESPGPGT
STPESGSASPGSTSESPSGTAPGSTSESPSGTAPGTSTPESGSASPGTST
PESGSASPGSEPATSGSETPGTSESATPESGPGSPAGSPTSTEEGTSTEP
SEGSAPGTSESATPESGPGTSTEPSEGSAPGTSTEPSEGSAPGSPAGSPT
STEEGTSTEPSEGSAPGTSTEPSEGSAPGTSESATPESGPGTSESATPES
GPGTSTEPSEGSAPGTSTEPSEGSAPGTSESATPESGPGTSTEPSEGSAP
GSEPATSGSETPGSPAGSPTSTEEGSSTPSGATGSPGTPGSGTASSSPGS
STPSGATGSPGTSTEPSEGSAPGTSTEPSEGSAPGSEPATSGSETPGSPA
GSPTSTEEGSPAGSPTSTEEGTSTEPSEGSAPGASASGAPSTGGTSESAT
PESGPGSPAGSPTSTEEGSPAGSPTSTEEGSTSSTAESPGPGSTSESPSG
TAPGTSPSGESSTAPGTPGSGTASSSPGSSTPSGATGSPGSSPSASTGTG
PGSEPATSGSETPGTSESATPESGPGSEPATSGSETPGSTSSTAESPGPG
STSSTAESPGPGTSPSGESSTAPGSEPATSGSETPGSEPATSGSETPGTS
TEPSEGSAPGSTSSTAESPGPGTSTPESGSASPGSTSESPSGTAPGTSTE
PSEGSAPGTSTEPSEGSAPGTSTEPSEGSAPGSSTPSGATGSPGSSPSAS
TGTGPGASPGTSSTGSPGSEPATSGSETPGTSESATPESGPGGSPAGSPT
STEEGSSTPSGATGSPGSSPSASTGTGPGASPGTSSTSPGTSESATPESG
PGTSTEPSEGSAPGTSTEPSEGSAP.

In another embodiment, the FPP peptide of AGP-FPP is a homologue of SEQ ID No: 59. In another embodiment, the FPP peptide of AGP-FPP is an analogue of SEQ ID No: 59. In another embodiment, the FPP peptide of AGP-FPP is a variant of SEQ ID No: 59. In another embodiment, the FPP peptide of AGP-FPP is any other XTEN peptide described in US Pat. Appl No. 20100323956 and are incorporated herein. Each possibility represents a separate embodiment of the present invention.

In another embodiment, the sequence of the FPP peptide of AGP-FPP is:

(Seq ID No. 60)
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGVGVPGVGVPGGGVPGAGVPGVGVPGVGVPGVGVPGGGVPGAGVPGGG
VPGWP.

In another embodiment, the FPP peptide of AGP-FPP is a homologue of SEQ ID No: 60. In another embodiment, the FPP peptide of AGP-FPP is an analogue of SEQ ID No: 60. In another embodiment, the FPP peptide of AGP-FPP is a variant of SEQ ID No: 60. In another embodiment, the FPP peptide of AGP-FPP is any other elastin like peptide (ELT) described in US Pat. Appl No. 20110178017 and US Pat. Appl. No. 20080032400 and are incorporated herein. Each possibility represents a separate embodiment of the present invention.

In another embodiment, the FPP peptide of AGP-FPP is an Fc fragment of an antibody. In another embodiment, the Fc fragment is derived from IgG. In another embodiment, the Fc fragment is selected from the IgG family, IgG1, IgG2, IgG3 and IgG4. In another embodiment, the Fc fragment of AGP-FPP is any mutated Fc peptide described in the art and incorporated herein. Each possibility represents a separate embodiment of the present invention.

In another embodiment, the AGP peptide of AGP-FPP is the precursor of Seq ID No. 1. In another embodiment, the precursor is metabolized in the subject's body to generate the active compound. In another embodiment, the active compound is generated via any other process known in the art. Each possibility represents a separate embodiment of the present invention.

In another embodiment, the AGP peptide of AGP-FPP of methods and compositions of the present invention is a mimetic of GLP-1. In another embodiment, the antagonist is a mimetic of Ex9-39. In another embodiment, the mimetic is an antagonist of a GLP-1R. In another embodiment, the mimetic exhibits protease resistance relative to GLP-1. In another embodiment, the mimetic exhibits protease resistance relative to a GLP-1 fragment (e.g. the GLP-1 fragment upon which the mimetic was modeled). In another embodiment, the mimetic is resistant to degradation by DPPIV. Each possibility represents another embodiment of the present invention.

In another embodiment, the AGP of AGP-FPP of the present invention is derived from an exendin peptide or GLP-1 peptide by incorporating 1 or more modified AA residues. In another embodiment, one or more of the termini is derivatized to include a blocking group, i.e. a chemical substituent suitable to protect and/or stabilize the N- and C-termini from undesirable degradation. In another embodiment, “undesirable degradation” refers to any type of enzymatic, chemical or biochemical breakdown of the compound at its termini which is likely to affect the function of the compound, i.e. sequential degradation of the compound at a terminal end thereof.

In another embodiment, blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino AA analogs are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkyl amino groups such as methyl amino, ethylamino, dimethylamino, diethylamino, methylethylamino and the like are examples of C-terminal blocking groups. Descarboxylated AA analogues such as agmatine are also useful C-terminal blocking groups and can be either coupled to the peptide's C-terminal residue or used in place of it. In another embodiment, the free amino and carboxyl groups at the termini are removed altogether from the peptide to yield desamino and descarboxylated forms thereof without affect on peptide activity.

In another embodiment, a mimetic compound of the present invention is derived from an exendin peptide or GLP-1 peptide by another modification. In another embodiment, such modifications include, but are not limited to, substitution of 1 or more of the AA in the natural L-isomeric form with D-isomeric AA. In another embodiment, the peptide includes one or more D-amino acid resides, or comprises AA that are all in the D-form. Retro-inverso forms of peptides in accordance with the present invention are also contemplated, for example, inverted peptides in which all AA are substituted with D-amino acid forms.

In another embodiment, the AGP-FPP of the present invention are produced by a process comprising the step of in vivo or in vitro chemical derivatization of the peptide, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. In another embodiment, a mimetic compound of the present invention comprises a phosphorylated AA residue, e.g., phosphotyrosine, phosphoserine, or phosphothreonine.

Methods of identifying AGP-FPP fusion protein mimetics are well known in the art, and are described, for example, in Song J et al, Biochem Cell Biol 76(2-3): 177-188, 1998; Vogt A et al, J Biol Chem. 270(2): 660-4, 1995; Alexopoulos K et al, J Med Chem 47(13): 3338-52, 2004; Andronati S A et al, Curr Med Chem 11(9): 1183-211, 2004; Breslin M J et al, Bioorg Med Chem Lett 13(10): 1809-12, 2003; and WO 02/081649 (“ErbB interface peptidomimetics and methods of use thereof”) in the name of Greene et al. In another embodiment, model building is used to design the mimetic compounds as described in one of the above references. In another embodiment, solubility of the mimetic compounds is optimized as described in one of the above references. Each possibility represents a separate embodiment of the present invention.

In another embodiment, the subject of methods and compositions of the present invention is a human subject. In another embodiment, the subject is a pediatric subject. In another embodiment, the subject is a child. In another embodiment, the subject is a juvenile. In another embodiment, the subject is a baby. In another embodiment, the subject is an infant. In another embodiment, the subject is an adolescent. In another embodiment, the subject is an adult. In another embodiment, the subject is any other type of subject known in the art. Each possibility represents a separate embodiment of the present invention.

In another embodiment, the subject is under 10 years of age. In another embodiment, the age is under 9 years. In another embodiment, the age is under 8 years. In another embodiment, the age is under 7 years. In another embodiment, the age is under 6 years. In another embodiment, the age is under 5 years. In another embodiment, the age is under 4 years. In another embodiment, the age is under 3 years. In another embodiment, the age is under 2 years. In another embodiment, the age is under 18 months. In another embodiment, the age is under 1 year. In another embodiment, the age is under 10 months. In another embodiment, the age is under 8 months. In another embodiment, the age is under 6 months. In another embodiment, the age is under 4 months. In another embodiment, the age is under 3 months. In another embodiment, the age is under 2 months. In another embodiment, the age is under 1 month.

In another embodiment, the age is over 6 months. In another embodiment, the age is over 1 year. In another embodiment, the age is over 2 years. In another embodiment, the age is over 3 years. In another embodiment, the age is over 5 years. In another embodiment, the age is over 7 years. In another embodiment, the age is over 10 years. In another embodiment, the age is over 15 years. In another embodiment, the age is over 20 years. In another embodiment, the age is over 30 years. In another embodiment, the age is over 40 years. In another embodiment, the age is over 50 years. In another embodiment, the age is over 60 years. In another embodiment, the age is over 65 years. In another embodiment, the age is over 70 years.

In another embodiment, the age is 1 month-5 years. In another embodiment, the age is 2 months-5 years. In another embodiment, the age is 3 months-5 years. In another embodiment, the age is 4 months-5 years. In another embodiment, the age is 6 months-5 years. In another embodiment, the age is 9 months-5 years. In another embodiment, the age is 1-5 years. In another embodiment, the age is 2-5 years. In another embodiment, the age is 3-5 years. In another embodiment, the age is 1-10 years. In another embodiment, the age is 1-5 years. In another embodiment, the age is 2-10 years. In another embodiment, the age is 3-10 years. In another embodiment, the age is 5-10 years. In another embodiment, the age is 1-6 months. In another embodiment, the age is 2-6 months. In another embodiment, the age is 3-12 months. In another embodiment, the age is 6-12 months.

Each age and age range represents a separate embodiment of the present invention.

### Pharmaceutical Formulations

In one embodiment, the invention provides a pharmaceutical formulation comprising an AGP-FPP fusion protein in admixture with a pharmaceutically acceptable carrier. The pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249: 1527-1533 (1990).

The pharmaceutical compositions can be administered by a number of routes, for instance, the parenteral, subcutaneous, intravenous, intranasal, topical, oral or local routes of administration, such as by aerosol or transdermally, for prophylactic and/or therapeutic treatment. Commonly, the pharmaceutical compositions may be administered parenterally, e.g., intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's, or fixed oils, intravenous vehicles include fluid and nutrient replenishers, electrolyte replenishers (such as those based on Ringer's dextrose), and the like.

Preservatives and other additives may also be present such as, for example, antimicrobials, anti-oxidants, chelating agents, and inert gases and the like. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

The compositions containing the glycolipid compounds can be administered for prophylactic and/or therapeutic treatments. In therapeutic applications, compositions are administered to a subject already suffering from a disease, as described above, in an amount sufficient to cure or at least partially arrest the symptoms of the disease and its complications. Amounts effective for this use will depend, as discussed further below, on the particular compound, the severity of the disease and the weight and general state of the subject, as well as the route of administration, but generally range from about 0.5 mg to about 4,000 mg of substrate per day for a 70 kg subject, with dosages of from about 5 mg to about 500 mg of the compounds per day being more commonly used.

In prophylactic applications, compositions containing the compound for use according to the invention are administered to a subject susceptible to or otherwise at risk of a particular disease. Such an amount is defined to be a “prophylactically effective dose.” In this use, the precise amounts again depend on the subject's state of health and weight, and the route of administration but generally range from about 0.5 mg to about 4,000 mg per 70 kilogram subject, more commonly from about 5 mg to about 500 mg per 70 kg of body weight.

Single or multiple administrations of the compositions can be carried out with dose levels and pattern being selected by the treating physician. In any event, the pharmaceutical formulations should provide a quantity of the substrates of this invention sufficient to effectively treat the subject.

Labeled substrates can be used to determine the locations at which the substrate becomes concentrated in the body due to interactions between the desired amino acid determinant and the corresponding ligand. For this use, the compounds can be labeled with appropriate radioisotopes, for example, ¹²⁵I, ¹⁴C, or tritium, or with other labels known to those of skill in the art.

The dosage ranges for the administration of the compounds for use according to the invention are those large enough to produce the desired effect. Generally, the dosage will vary with the age, condition, sex and extent of the disease in the subject and can be determined by one of skill in the art. The dosage can be adjusted by the individual physician monitoring the therapy.

In some embodiments, compounds of the present invention are administered by intravenous infusion at a rate ranging from about 80 pmol/kg/min to about 600 pmol/kg/min, from about 100 pmol/kg/min to about 580 pmol/kg/min, from about 120 pmol/kg/min to about 560 pmol/kg/min, from about 140 pmol/kg/min to about 540 pmol/kg/min, from about 160 to about 520 pmol/kg/min, from about 180 pmol/kg/min to about 500 pmol/kg/min, from about 200 pmol/kg/min to about 480 pmol/kg/min, from about 220 pmol/kg/min to about 460 pmol/kg/min, from about 240 pmol/kg/min to about 440 pmol/kg/min, from about 260 pmol/kg/min to about 420 pmol/kg/min, from about 280 pmol/kg/min to about 400 pmol/kg/min, from about 300 pmol/kg/min to about 380 pmol/kg/min, from about 320 pmol/kg/min to about 360 pmol/kg/min. In other embodiments, compounds of the present invention are administered by intravenous infusion at a rate ranging from about 80-100 pmol/kg/min, from about 100-120 pmol/kg/min, from about 120-140 pmol/kg/min, from about 140-160 pmol/kg/min, from about 160-180 pmol/kg/min, from about 180-200 pmol/kg/min, from about 180-200 pmol/kg/min, from about 200-220 pmol/kg/min, from about 220-240 pmol/kg/min, from about 240-260 pmol/kg/min, from about 260-280 pmol/kg/min, from about 280-300 pmol/kg/min, from about 300-320 pmol/kg/min, from about 320-340 pmol/kg/min, from about 340-360 pmol/kg/min, from about 360-380 pmol/kg/min, from about 380-400 pmol/kg/min, from about 400-420 pmol/kg/min, from about 420-440 pmol/kg/min, from about 440-460 pmol/kg/min, from about 460-480 pmol/kg/min, from about 480-500 pmol/kg/min, from about 500-520 pmol/kg/min, from about 520-540 pmol/kg/min, from about 540-560 pmol/kg/min, from about 560-580 pmol/kg/min, from about 580-600 pmol/kg/min.

Additional pharmaceutical methods may be employed to control the duration of action. Controlled release preparations may be achieved by the use of polymers to conjugate, complex or adsorb the fusion proteins. The controlled delivery may be exercised by selecting appropriate macromolecules (for example, polyesters, polyamino carboxymethylcellulose, and protamine sulfate) and the concentration of macromolecules as well as the methods of incorporation in order to control release. Another possible method to control the duration of action by controlled release preparations is to incorporate the fusion protein into particles of a polymeric material such as polyesters, polyamino acids, hydrogels, poly (lactic acid) or ethylene vinylacetate copolymers. In one embodiment, the compositions providea controlled release of an oral administered composition in the lower GI tract or intestines.

The compounds for use according to the invention are well suited for use in targetable drug delivery systems such as synthetic or natural polymers in the form of macromolecular complexes, nanocapsules, microspheres, or beads, and lipid-based systems including oil-in-water emulsions, micelles, mixed micelles, liposomes, and resealed erythrocytes. These systems are known collectively as colloidal drug delivery systems. Typically, such colloidal particles containing the dispersed glycosphingolipids are about 50 nm-2 microns in diameter. The size of the colloidal particles allows them to be administered intravenously such as by injection, or as an aerosol. Materials used in the preparation of colloidal systems are typically sterilizable via filter sterilization, nontoxic, and biodegradable, for example albumin, ethylcellulose, casein, gelatin, lecithin, phospholipids, and soybean oil. Polymeric colloidal systems are prepared by a process similar to the coacervation of microencapsulation.

The compositions of this invention can be prepared in any suitable formulation now known or hereafter developed, including, but not limited to, ampoules, creams, ointments, gels, pellets, patches or solutions, in a pharmacologically acceptable carrier. The invention is administered to a patient by various suitable means now known or hereafter developed, including, but not limited to, topical delivery, subcutaneous or intralesional, intramuscular, transcutaneous and transdermal delivery, intravenous, or gene therapy.

Suitable acceptable carriers for a topical formulation can be water, salt solutions, alcohols, oils, glycols, gelatine, carbohydrates such as lactose, amylose or starch, fatty acid monoglycerides and diglycerides, pentaerythritol fatty acid esters, hydroxy methylcellulose, polyvinyl pyrrolidone, etc. The preparations can be sterilized and if desired mixed with auxiliary agents, e.g., lubricants, preservatives, stabilizers, wetting agents, emulsifiers, salts for influencing osmotic pressure, buffers, coloring, aromatic substances and the like that do not deleteriously react with the active compounds. They can also be combined where desired with other active agent.

### EXPERIMENTAL DETAILS

The invention is further described with reference to the following Examples. The Examples are provided for the purpose of illustration only and the invention not be construed as being limited to these Examples, but rather should be construed to encompass any and all variations which become evident as a result of the teaching provided herein.

The compositions of the present invention are tested for therapeutic efficacy in well established rodent models of Congentical Disease (e.g. Familial Hyperinsulinemia) which are considered to be representative of a human disease. The overall approaches are described in detail in Koster, Proc Natl Acad Sci USA, 99:16992-16997 (2002); Remedi, Diabetologia, 49:2368-2378 (2006); Marshall, J Biol Chem, 274:27426-27432 (1999); US Patent Application No. 20080269130; and, Machado, Biol Pharm Bull, 32:232-236 (2009). These references are hereby incorporated by reference in their entirety. An exemplary example is the use of SUR1−/− mice to evaluate the ability of the AGP-FPP fusion proteins to increase fasting glucose levels.

### Example 1

### Cloning of AGP-ELP Constructs

The DNA sequence for the AGP-ELP fusion constructs is codon opotimized, made synthetically (Genewiz, Inc.) and the DNA sequence incorporated into the PET24a, PB1046 and pPB1031 vectors.

### Example 2

### Expression of AGP-ELP

The E. coli production strain BLR (Novogen) is transformed with the plasmids PET24a, PB 1046 and pPB 1031 and grown in rich medium in shake flasks at 37° C. overnight. The cell pellets is resuspended in TE pH 8.0 buffer, lysed through a microfluidizer, centrifuged to remove the insoluble material and the product purified from the resulting soluble lysate by ‘transitioning’ with the addition of NaCl to 3M (Hassouneh et al, Curr Protoc Protein Sci, Chapter 6, Unit 6.11, 2010). The samples is taken through a further two rounds of transitioning to give the final purified samples. These are analyzed by SDS-PAGE.

### Example 3

### Expression of AGP-FPP

The DNA sequence for the AGP-FPP fusion construct is codon optimized, made synthetically (Genewiz, Inc.) and the DNA sequence incorporated into the PET24a, PB1046 and pPB1031 vectors.

### Example 4

### Expression of AGP-FPP

The E. coli production strain BLR (Novogen) is transformed with the plasmids PET24a, grown in rich medium in shake flasks at 37° C. overnight and induced with IPTG. The cell pellets is resuspended in Tris pH 8.0 buffer, lysed through a microfluidizer, centrifuged to remove the insoluble material and the product purified using two chromatography steps, Source 15Q and butyl sepharose FF. The samples are analyzed by SDS-PAGE and SEC chromatography (TSK-gel, G3000 SWXL).

### Example 5

### Glucose and Insulin Tolerance Test

SUR1−/− mice are treated with AGP-FPP selected from Seq ID No. 19 to 21 and Seq ID No. 50 to 55), subcutaneouse administration. A glucose tolerance test is performed by administering 2 g/kg of dextrose (oral gavage) and then measuring blood glucose levels after fasting for 12-16 hours. The insulin tolerance test is performed by administering 0.5 units/Kg (intraperitoneally) of insulin to the mice after a 4 hour fast. Blood glucose levels are measured using a glucose me Islet Studies

### Example 6

### Insulin Release Assay

Islets are isolated by collagenase digestion and cultured for 3 days in RPMI 1640 medium containing 10 mM glucose. The culture medium is supplemented with 10% fetal bovine serum, 2 mM glutamine, 100 units/mL penicillin, and 50 micrograms/mL streptomycin. Islets are incubated at 37° C. in a 5% CO₂, 95% air-humidified incubator. Batches of 100 cultured mouse islets are loaded onto a nylon filter in a chamber and perifused with Krebs-Ringer bicarbonate buffer (115 mM NaCl, 24 mM NaHCO₃, 5 mM KCl, 1 mM MgCl₂, 2.5 mM CaCl₂, 10 mM HEPES, pH 7.4) with 0.25% bovine serum albumin at a flow rate of 2 mL/min. Perifusate solutions are gassed with 95% O2, 5% CO₂ and maintained at 37° C. Islets are stimulated with a ramp of amino acids. The physiologic mixture of 19 amino acids is used at a maximum concentration of 12 mM with the following composition (in mM): glutamine 2, alanine 1.25, arginine 0.53, aspartate 0.11, citrulline 0.27, glutamate 0.35, glycine 0.85, histidine 0.22, isoleucine 0.27, leucine 0.46, lysine 1.06, methionine 0.14, ornithine 0.20, phenylalanine 0.23, proline 1, serine 1.62, threonine 0.77, tryptophan 0.21, valine 0.57. Samples are collected every minute for insulin assays. Insulin is measured by radioimmunoassay.

### Example 7

### cAMP Content Test

Islets are isolated as above and cultured for three days. Cultured islets are preincubated in glucose free Krebs-Ringer bicarbonate buffer for 60 min, 1 mM AGP-FPP (selected from Seq ID No. 19 to 21 and 50-55) is added 30 min into the preincubation period. Then, islets are exposed to different treatments for an additional 30 min in the presence of 0.1 mM isobutyl-methylzanthine (IBMX). After incubation, islets are washed 2 times by cold glucose-free Hank's buffer. cAMP is measured in islet lysates by ELISA.

### Example 8

### Cytosolic Free Ca2+ Measurements

Mouse islets are isolated and cultured on poly-Lysine coated glass coverslips under the same conditions as described above. In brief, the coverslip with attached islets is incubated with 15 mM Fura-2 acetoxymethylester in Krebs-Ringer bicarbonate buffer with 5 mM glucose for 35 min at 37° C. Islets are then perifused with Krebs-Ringer bicarbonate buffer with 0.25% bovine serum albumin at 37° C. at a flow rate of 2 mL/min, while various agents were applied. [Ca²⁺]ᵢ was measured with a dual wavelength fluorescence microscope.

### Example 9

### Pharmacokinetic Determination

The pharmacokinetic actions of the compounds for use according to the invention can be studied by determining blood levels of the administered AGP-FPP over time. For this purpose, radiolabeled compounds for use according to the invention may be especially suitable. Methods for identifying and quantifying such compounds in samples are as set forth above. In some embodiments, the improved pharmacokinetic properties are assessed in a test species of mammal (e.g., mouse, rat, rabbit, pig, primate) or in clinical studies. Improved pharmacokinetics include better distribution to a target organs and tissues (PNS, CNS, blood tissues, nerve, blood cells) and improved half-lives. Alternatively, the blood levels of the administered AGP-FPP are measured using an ELISA assay using antibodies directed to either the AGP- or FPP portions of the fusion proteins.

### Example 10

### Effect of AGPL-FPP fusion Proteins in Regulating Insulin and Plasma Glucose Levels in HI Patients

After an overnight fast, subject receives an intravenous infusion or subcutaneous injection of a fusion protein selected from Seq ID No. 19 to 55. On the second day, the subject is fasted overnight. Blood samples for glucose, insulin, C-peptide, and glucagon are obtained at different intervals after compound administration.

### Example 11

### Effect of AGP Fusion Proteins in Mice Studies

Animals. An animal model harboring a targeted inactivation of the SUR-1 gene (SUR-1^{-/-} mouse) reproduces the key pathophysiological features of K_{ATP}HI. The generation and genotyping of SUR-1^{-/-} mice are previously described (7). In this study, mice are maintained in a C57B1/6 genetic background. 12-18-week SUR-1^{-/-} and wild-type littermate control mice are used in all experiments. Mice are maintained on a 12/12-h light/dark cycle and were fed a standard rodent chow diet. All procedures are approved and carried out according to the University of Pennsylvania Institutional Animal Care and Use Committee guidelines.

Exendin-(9-39) Administration.

Alzet miniosmotic pumps (model 2002; Alza, Palo Alto, Calif.) are implanted subcutaneously to deliver exendin-(9-39) (Bachem Bioscience, King of Prussia, Pa.) at a rate of 150 pmol/kg/min or vehicle (0.9% NaCl, 1% bovine serum albumin) for 2 weeks.

Glucose Homeostasis.

For determination of fasting blood glucose levels, mice are fasted for 12-16 h. Oral glucose tolerance testing is carried after a 12-16-h fast by administering 2 g/kg of dextrose by oral gavage (feeding needles; Popper and Sons, Inc., Hyde Park, N.Y.). For insulin tolerance testing, mice receive 0.5 units/kg of insulin intraperitoneally after a 4-h fast. Blood glucose levels are measured using a hand-held glucose meter (FreeStyle; TheraSense, Alameda, Calif.). Insulin and glucagon are measured by ELISA (Mouse Endocrine Immunoassay Panel; Linco Research, Inc., St. Charles, Mo.).

Islet Studies.

Islets are isolated by collagenase digestion and cultured for 3 days in RPMI 1640 medium containing 10 mM glucose. The culture medium is supplemented with 10% fetal bovine serum, 2 mM glutamine, 100 units/ml penicillin, and 50 μg/ml streptomycin. Islets are incubated at 37° C. in a 5% CO2, 95% air-humidified incubator. Batches of 100 cultured mouse islets are loaded onto a nylon filter in a chamber and perifused with Krebs-Ringer bicarbonate buffer (115 mM NaCl, 24 mM NaHCO₃, 5 mM KCl, 1 mM MgCl₂, 2.5 mM CaCl₂, 10 mM HEPES, pH 7.4) with 0.25% bovine serum albumin at a flow rate of 2 ml/min. Perifusate solutions are gassed with 95% O₂, 5% CO2 and maintained at 37° C. Islets are stimulated with a ramp of amino acids. The mixture of 19 amino acids when used at a maximum concentration of 12 mM (about 3 times physiological concentration) have the following composition: 2 mM glutamine, 1.25 mM alanine, 0.53 mM arginine, 0.11 mM aspartate, 0.27 mM citrulline, 0.35 mM glutamate, 0.85 mM glycine, 0.22 mM histidine, 0.27 mM isoleucine, 0.46 mM leucine, 1.06 mM lysine, 0.14 mM methionine, 0.20 mM ornithine, 0.23 mM phenylalanine, 1 mM proline, 1.62 mM serine, 0.77 mM threonine, 0.21 mM tryptophan, 0.57 mM valine. Samples are collected every minute for insulin assays. Insulin is measured by radioimmunoassay (Linco Research Inc., St. Charles, Mo.).

cAMP Content Determination.

Islets are isolated as above, hand-picked, and cultured for 3 days. Cultured islets are pre-incubated in glucose-free Krebs-Ringer bicarbonate buffer for 60 min, and 100 nM exendin—(9-39) is added 30 min into the preincubation period. Then, islets are exposed to different treatments for an additional 30 min in the presence of 0.1 mM isobutylmethylzanthine. After incubation, islets are washed two times by cold glucose-free Hanks' buffer. cAMP is measured in islet lysates by an enzyme-linked immunosorbent assay (GE Healthcare).

Cytosolic Free Ca²⁺ Measurements.

Mouse islets are isolated and cultured on poly-L-lysine-coated glass coverslips under the same conditions as described above. The perifusion procedure and cytosolic-free Ca²⁺ ([Ca²⁺]ᵢ) measurement are described previously (23). In brief, the coverslip with attached islets is incubated with 15 μM Fura-2 acetoxymethylester (Molecular Probes, Inc., Eugene, Oreg.) in Krebs-Ringer bicarbonate buffer with 5 mM glucose for 35 min at 37° C. Islets are then perifused with Krebs-Ringer bicarbonate buffer with 0.25% bovine serum albumin at 37° C. at a flow rate of 2 ml/min while various agents are applied. [Ca²⁺]ᵢ is measured with a dual wavelength fluorescence microscope as previously described.

Statistical Evaluation.

Data presented are mean±S.E. and compared using Student's t test. For glucose and insulin tolerance testing, values were compared by repeated measures ANOVA.¹ Differences are considered significant at p<0.05. ¹ The abbreviation used is: ANOVA, analysis of variance.

### Example 12

### Effect of AGP Fusion Proteins in Human Studies

Research Design and Methods.

Nine subjects with confirmed genetic and clinical diagnosis of KATP hyperinsulinism are recruited from the Hyperinsulinism Center at the Children's Hospital of Philadelphia (CHOP). Exclusion criteria include acute medical illnesses; a history of systemic chronic diseases such as cardiac failure, renal insufficiency, hepatic insufficiency, chronic obstructive pulmonary disease, anemia, or uncontrolled hypertension; pregnancy; diabetes; and use of medications that affect glucose metabolism, such as glucocorticoids, P-agonists, octreotide, and diazoxide.

This is a randomized, open-label, two-period complete crossover pilot study to evaluate the effect of the GLP-1 receptor antagonist exendin—(9-39), on glucose metabolism in subjects with K_{ATP}HI. All subjects are administered 5 ng exendin(9-39) (0.05 μg/mL) intradermally as a test of immediate hypersensitivity. Baseline chemistry profiles are obtained to evaluate liver and kidney function in all subjects, and a pregnancy test is performed in all postmenarchal females.

An antecubital vein is cannulated in each forearm for infusions and blood sampling. Each subject undergo two experiments in random order and on consecutive days. On one day, after a 12-h overnight fast, subjects receive an intravenous infusion of vehicle (0.9% NaCl) for 1 h followed by an intravenous infusion of exendin-(9-39) at 100 pmol/kg/min (0.02 mg/kg/h) for 2 h and then 300 pmol/kg/min (0.06 mg/kg/h) for 2 h, followed by 500 pmol/kg/min (0.1 mg/kg/h) for the last 2 h. The doses of exendin-(9-39) are selected based on previously published data demonstrating that at a dose of 300 pmol/kg/min, exendin-(9-39) abolishes the effects of physiologic postprandial plasma concentrations of GLP-1 and that a higher dose of 500 pmol/kg/min increases fasting plasma glucose concentration in normal subjects (5,12). On the other day, after a 12-h overnight fast, subjects receive an intravenous infusion of vehicle for 7 h. The infusion rates of vehicle are identical to the volume infused during the exendin-(9-39) study day. The primary outcome for this study is fasting blood glucose concentration. Secondary outcomes include fasting plasma insulin, C-peptide, glucagon, intact GLP-1, and insulin/glucose. Blood samples for blood glucose, insulin, glucagon, and intact GLP-1 are obtained at multiple time points during the infusions (−60, 0, 40, 60, 80, 120, 160, 180, 200, 220, 240, 280, 300, 320, 340, and 360 min). During the infusion, blood glucose is monitored by a bedside glucose meter (Surestep) as needed to avoid hypoglycemia (defined as <3.9 mmol/L [70 mg/dL]). An intravenous infusion of dextrose is started if blood glucose levels fall to <3.3 mmol/L (60 mg/dL) during the study period.

Peptide.

Exendin-(9-39) is synthesized by the American Peptide Company (Sunnyvale, Calif.) under cGMP guidelines. The peptide is purified to >97% by high-performance liquid chromatography, and the sequence and mass were verified. The peptide is stored in a lyophilized form at −20° C. For administration, the peptide is diluted in 0.9% NaCl and added to 0.25% human serum albumin (final concentration of 0.1 mg/mL). Aliquots are tested for sterility and pyrogenicity through the Investigational Drug Service at the University of Pennsylvania. The use of synthetic exendin-(9-39) is approved under the U.S. Food and Drug Administration Investigational New Drug no. 76612.

Islet Studies.

Fresh pancreata from surgical specimens from three neonates (age 4-6 weeks) with K_{ATP}HI who are homozygous for mutations in either KCNJ11 (R136L) or ABCC8 (R248X and E824X) are procured through an institutional review board-approved protocol. The pancreas is injected with collagenase (Sigma-Aldrich; St. Louis, Mo.). Islets are handpicked under microscopy and cultured in RPMI-1640 medium containing 10 mmol/L glucose for 3 days prior to the studies. Batches of 50 islets are preincubated in glucose-free Krebs-Ringer bicarbonate buffer for 60 min. Exendin-(9-39) is added 30 min into the preincubation period. Then, islets are exposed to stimulation with 10 mmol/L glucose or a mixture of amino acids at a concentration of 4 mmol/L as previously described (8). Media are collected for determination of insulin concentration.

Assays.

Whole blood glucose was measured using a Siemens Rapid Point 400 Blood Gas analyzer (Siemens Healthcare Diagnostics, Deerfield, Ill.). The analyzer has a resolution of 1 mg/dL and a within-run SD of ±4 mg/dL. Plasma insulin was measured using an ELISA kit from ALPCO (cat. no. 08-10-1113-99; ALPCO Diagnostics, Salem, N.H.). The assay has a sensitivity of 0.798 uIU/mL and an intra-assay coefficient of variation (CV) of <5%. C-peptide was measured using an RIA kit (cat. no. HCP-20K, Millipore; Linco Research, St. Charles, Mo.). The assay has a sensitivity of 0.1 ng/mL and an intra-assay CV of <10%. Glucagon is measured using an RIA kit (cat. no. GL-32K, Millipore; Linco Research). The assay has a sensitivity of 20 μg/mL and an intra-assay CV of <10%. Intact GLP-1 is measured using a GLP-1 ELISA kit (cat. no. EGLP35K, Millipore; Linco Research) in samples collected with dipeptidyl peptidase IV inhibitor (cat. no. DPP4, Millipore; Linco Research) (10 mL/mL blood) to prevent proteolytic cleavage. The kit has a sensitivity of 2 pmol/L and an intraassay CV of <10%. Insulin concentrations from the islet studies are measured by RIA (Millipore; Linco Research).

Statistical Analysis.

All results are presented as means±SD. Area under the plasma concentration-time curve (AUC) is calculated for each outcome, under each treatment condition, using the linear trapezoid method. Histograms and one-sample Kolmogorov-Smirnov tests are used to examine outcome variables for normality of distribution. Effects of carryover, period, and treatment are examined using mixed-effects models (SAS proc mixed). Results from the islet studies are analyzed by one-way ANOVA.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes to the extent not inconsistent with the present disclosure.

TABLE 1
Amino Acid Sequences of Exemplary Antagonists of
the GLP-1 Receptor.

| | Seq |
|---|---|
| | ID |
| Analog Sequence | No: |
| DLSKQMEEEAVRLFIEWLKNGGPSSGAPPPS | 1 |
| HAEGTFTSDVSSYLEGQAAKEFIAAWLVKGR | 2 |
| HGEGTFTSDLSKQMEEEAVRLFIEWLKNGGPSSGAPPPS | 3 |
| GEGTFTWELSKQMEEEAVRLFIEWLKNGGPSSGAPPPS | 4 |
| GEGTFTSQLSKQMEEEAVRLFIEWLKNGGPSSGAPPPS | 5 |
| KRHSDGTFTSDLSKQMEEEAVRLFIEWLKNGGPSSGAPPPS | 6 |
| HSDGTFSDLSKGMEEEAVRLHEWLKNGGPSSGAPPPS | 7 |
| HGEGTFTSDLSKQMEEEAVRLFIEWLKNGGPSSGAPPPS | 8 |
| HAEGTFTSKVSSYLEGQAAKEFIAWLVKGR | 9 |
| GEGTFTSDLSKQMEEEAVRLFIEWLKNGGPSSGAPPPS | 10 |
| EGTFTSDLSKQMEEEAVRLFIEWLKNGGPSSGAPPPS | 11 |
| GTFTSDLSKQMEEEAVRLFIEWLKNGGPSSGAPPPS | 12 |
| MKIILWLCVFGLFLATLFPVSWQMPVESGLSSEDSASSESFASKI | 13 |
| KRHSDGTFTSDLSKQMEEEAVRLFIEWLKNGGPSSGAPPPSG | |
| SDLSKQMEEEAVRLFIEWLKNGGPSSGAPPPS | 14 |
| TFTSDLSKQMEEEAVRLFIEWLKNGGPSSGAPPPS | 15 |
| FTSDLSKQMEEEAVRLFIEWLKNGGPSSGAPPPS | 16 |
| TSDLSKQMEEEAVRLFIEWLKNGGPSSGAPPPS | 17 |
| GEGTFTSELSKQMEEEAVRLFIEWLKNGGPSSGAPPPS | 18 |

TABLE 2
Fusion Protein Partners.

| Sequence | Seq ID No. |
|---|---|
| VPDKTVRWCAVSEHEATKCQSFRDHMKSVIPSDGPSVACVKKASYL | 56 |
| DCIRAIAANEADAVTLDAGLVYDAYLAPNNLKPVVAEFYGSKEDPQT | |
| FYYAVAVVKKDSGFQMNQLRGKKSCHTGLGRSAGWNIPIGLLYCDL | |
| PEPRKPLEKAVANFFSGSCAPCADGTDFPQLCQLCPGCGCSTLNQYFG | |
| YSGAFKCLKDGAGDVAFVKHSTIFENLANKADRDQYELLCLDNTRKP | |
| VDEYKDCHLAQVPSHTVVARSMGGKEDLIWELLNQAQEHFGKDKSK | |
| EFQLFSSPHGKDLLFKDSAHGFLKVPPRMDAKMYLGYEYVTAIRNLR | |
| EGTCPEAPTDECKPVKWCALSHHERLKCDEWSVNSVGKIECVSAETT | |
| EDCIAKIMNGEADAMSLDGGFVYIAGKCGLVPVLAENYNKSDNCEDT | |
| PEAGYFAIAVVKKSASDLTWDNLKGKKSCHTAVGRTAGWNIPMGLL | |
| YNKINHCRFDEFFSEGCAPGSKKDSSLCKLCMGSGLNLCEPNNKEGY | |
| YGYTGAFRCLVEKGDVAFVKHQTVPQNTGGKNPDPWAKNLNEKDY | |
| ELLCLDGTRKPVEEYANCHLARAPNHAVVTRKDKEACVHKILRQQQ | |
| HLFGSNVTDCSGNFCLFRSETKDLLFRDDTVCLAKLHDRNTYEKYLG | |
| EEYVKAVGNLRKCSTSSLLEACTFRRP | |
| DAHKSEVAHRFKDLGEENFKALVLIAFAQYLQQCPFEDHVKLVNEVT | 57 |
| EFAKTCVADESAENCDKSLHTLFGDKLCTVATLRETYGEMADCCAK | |
| QEPERNECFLQHKDDNPNLPRLVRPEVDVMCTAFHDNEETFLKKYLY | |
| EIARRHPYFYAPELLFFAKRYKAAFTECCQAADKAACLLPKLDELRDE | |
| GKASSAKQRLKCASLQKFGERAFKAWAVARLSQRFPKAEFAEVSKL | |
| VTDLTKVHTECCHGDLLECADDRADLAKYICENQDSISSKLKECCEKP | |
| LLEKSHCIAEVENDEMPADLPSLAADFVESKDVCKNYAEAKDVFLGM | |
| FLYEYARRHPDYSVVLLLRLAKTYETTLEKCCAAADPHECYAKVFDE | |
| FKPLVEEPQNLIKQNCELFEQLGEYKFQNALLVRYTKKVPQVSTPTLV | |
| EVSRNLGKVGSKCCKHPEAKRMPCAEDYLSVVLNQLCVLHEKTPVS | |
| DRVTKCCTESLVNRRPCFSALEVDETYVPKEFNAETFTFHADICTLSE | |
| KERQIKKQTALVELVKHKPKATKEQLKAVMDDFAAFVEKCCKADDK | |
| ETCFAEEGKKLVAASQAALGL | |
| GSPAGSPTSTEEGTSESATPESGPGTSTEPSEGSAPGSPAGSPTSTEEGT | 58 |
| STEPSEGSAPGTSTEPSEGSAPGTSESATPESGPGSEPATSGSETPGSEPA | |
| TSGSETPGSPAGSPTSTEEGTSESATPESGPGTSTEPSEGSAPGTSTEPSE | |
| GSAPGSPAGSPTSTEEGTSTEPSEGSAPGTSTEPSEGSAPGTSESATPES | |
| GPGTSTEPSEGSAPGTSESATPESGPGSEPATSGSETPGTSTEPSEGSAP | |
| GTSTEPSEGSAPGTSESATPESGPGTSESATPESGPGSPAGSPTSTEEGT | |
| SESATPESGPGSEPATSGSETPGTSESATPESGPGTSTEPSEGSAPGTSTE | |
| PSEGSAPGTSTEPSEGSAPGTSTEPSEGSAPGTSTEPSEGSAPGTSTEPSE | |
| GSAPGSPAGSPTSTEEGTSTEPSEGSAPGTSESATPESGPGSEPATSGSE | |
| TPGTSESATPESGPGSEPATSGSETPGTSESATPESGPGTSTEPSEGSAP | |
| GTSESATPESGPGSPAGSPTSTEEGSPAGSPTSTEEGSPAGSPTSTEEGT | |
| SESATPESGPGTSTEPSEGSAPGTSESATPESGPGSEPATSGSETPGTSES | |
| ATPESGPGSEPATSGSETPGTSESATPESGPGTSTEPSEGSAPGSPAGSP | |
| TSTEEGTSESATPESGPGSEPATSGSETPGTSESATPESGPGSPAGSPTST | |
| EEGSPAGSPTSTEEGTSTEPSEGSAPGTSESATPESGPGTSESATPESGP | |
| GTSESATPESGPGSEPATSGSETPGSEPATSGSETPGSPAGSPTSTEEGT | |
| STEPSEGSAP | |
| GTSTEPSEGSAPGSEPATSGSETPGSPAGSPTSTEEGSTSSTAESPGPGTS | 59 |
| TPESGSASPGSTSESPSGTAPGSTSESPSGTAPGTSTPESGSASPGTSTPE | |
| SGSASPGSEPATSGSETPGTSESATPESGPGSPAGSPTSTEEGTSTEPSEG | |
| SAPGTSESATPESGPGTSTEPSEGSAPGTSTEPSEGSAPGSPAGSPTSTEE | |
| GTSTEPSEGSAPGTSTEPSEGSAPGTSESATPESGPGTSESATPESGPGT | |
| STEPSEGSAPGTSTEPSEGSAPGTSESATPESGPGTSTEPSEGSAPGSEPA | |
| TSGSETPGSPAGSPTSTEEGSSTPSGATGSPGTPGSGTASSSPGSSTPSG | |
| ATGSPGTSTEPSEGSAPGTSTEPSEGSAPGSEPATSGSETPGSPAGSPTS | |
| TEEGSPAGSPTSTEEGTSTEPSEGSAPGASASGAPSTGGTSESATPESGP | |
| GSPAGSPTSTEEGSPAGSPTSTEEGSTSSTAESPGPGSTSESPSGTAPGTS | |
| PSGESSTAPGTPGSGTASSSPGSSTPSGATGSPGSSPSASTGTGPGSEPA | |
| TSGSETPGTSESATPESGPGSEPATSGSETPGSTSSTAESPGPGSTSSTAE | |
| SPGPGTSPSGESSTAPGSEPATSGSETPGSEPATSGSETPGTSTEPSEGSA | |
| PGSTSSTAESPGPGTSTPESGSASPGSTSESPSGTAPGTSTEPSEGSAPGT | |
| STEPSEGSAPGTSTEPSEGSAPGSSTPSGATGSPGSSPSASTGTGPGASP | |
| GTSSTGSPGSEPATSGSETPGTSESATPESGPGGSPAGSPTSTEEGSSTPS | |
| GATGSPGSSPSASTGTGPGASPGTSSTSPGTSESATPESGPGTSTEPSEG | |
| SAPGTSTEPSEGSAP | |
| VPGVGVPGVGVPGGGVPGAGVPGVGVPGVGVPGVGVPGGGVPGAG | 60 |
| VPGGGVPGVGVPGVGVPGGGVPGAGVPGVGVPGVGVPGVGVPGGG | |
| VPGAGVPGGGVPGVGVPGVGVPGGGVPGAGVPGVGVPGVGVPGVG | |
| VPGGGVPGAGVPGGGVPGVGVPGVGVPGGGVPGAGVPGVGVPGVG | |
| VPGVGVPGGGVPGAGVPGGGVPGVGVPGVGVPGGGVPGAGVPGVG | |
| VPGVGVPGVGVPGGGVPGAGVPGGGVPGVGVPGVGVPGGGVPGAG | |
| VPGVGVPGVGVPGVGVPGGGVPGAGVPGGGVPGVGVPGVGVPGGG | |
| VPGAGVPGVGVPGVGVPGVGVPGGGVPGAGVPGGGVPGVGVPGVG | |
| VPGGGVPGAGVPGVGVPGVGVPGVGVPGGGVPGAGVPGGGVPGVG | |
| VPGVGVPGGGVPGAGVPGVGVPGVGVPGVGVPGGGVPGAGVPGGG | |
| VPGVGVPGVGVPGGGVPGAGVPGVGVPGVGVPGVGVPGGGVPGAG | |
| VPGGGVPGVGVPGVGVPGGGVPGAGVPGVGVPGVGVPGVGVPGGG | |
| VPGAGVPGGGVPGVGVPGVGVPGGGVPGAGVPGVGVPGVGVPGVG | |
| VPGGGVPGAGVPGGGVPGWP | |
| GSETATSGSETAGTSESATSESGAGSTAGSETSTEAGTSESATSESGAG | 61 |
| SETATSGSETAGSETATSGSETAGTSTEASEGSASGTSTEASEGSASGT | |
| SESATSESGAGSETATSGSETAGTSTEASEGSASGSTAGSETSTEAGTS | |
| ESATSESGAGTSESATSESGAGSETATSGSETAGTSESATSESGAGTST | |
| EASEGSASGSETATSGSETAGSETATSGSETAGTSTEASEGSASGSTAG | |
| SETSTEAGTSESATSESGAGTSTEASEGSASGSETATSGSETAGSTAGS | |
| ETSTEAGSTAGSETSTEAGSETATSGSETAGTSESATSESGAGTSESAT | |
| SESGAGSETATSGSETAGTSESATSESGAGTSESATSESGAGSETATSG | |
| SETAGSETATSGSETAGTSTEASEGSASGSTAGSETSTEAGSETATSGS | |
| ETAGTSESATSESGAGSTAGSETSTEAGSTAGSETSTEAGSTAGSETST | |
| EAGTSTEASEGSASGSTAGSETSTEAGSTAGSETSTEAGTSTEASEGSA | |
| SGSTAGSETSTEAGSETATSGSETAGTSTEASEGSASGTSESATSESGA | |
| GSETATSGSETAGTSESATSESGAGTSESATSESGAGSETATSGSETAG | |
| TSESATSESGAGSETATSGSETAGTSTEASEGSASGTSTEASEGSASGS | |
| TAGSETSTEAGSTAGSETSTEAGSETATSGSETAGTSESATSESGAGTS | |
| ESATSESGAGSETATSGSETAGSETATSGSETAGSETATSGSETAGTST | |
| EASEGSASGTSESATSESGAGSETATSGSETAGSETATSGSETAGTSES | |
| ATSESGAGTSESATSESGAGSETATSGSETA | |
| GTSTEPSEPGSAGTSTEPSEPGSAGSEPATSGTEPSGSGASEPTSTEPGS | 62 |
| EPATSGTEPSGSEPATSGTEPSGSEPATSGTEPSGSGASEPTSTEPGTSTE | |
| PSEPGSAGSEPATSGTEPSGTSTEPSEPGSAGSEPATSGTEPSGSEPATS | |
| GTEPSGTSTEPSEPGSAGTSTEPSEPGSAGSEPATSGTEPSGSEPATSGT | |
| EPSGTSEPSTSEPGAGSGASEPTSTEPGTSEPSTSEPGAGSEPATSGTEPS | |
| GSEPATSGTEPSGTSTEPSEPGSAGTSTEPSEPGSAGSGASEPTSTEPGS | |
| EPATSGTEPSGSEPATSGTEPSGSEPATSGTEPSGSEPATSGTEPSGTSTE | |
| PSEPGSAGSEPATSGTEPSGSGASEPTSTEPGTSTEPSEPGSAGSEPATS | |
| GTEPSGSGASEPTSTEPGTSTEPSEPGSAGSGASEPTSTEPGSEPATSGT | |
| EPSGSGASEPTSTEPGSEPATSGTEPSGSGASEPTSTEPGTSTEPSEPGSA | |
| GSEPATSGTEPSGSGASEPTSTEPGTSTEPSEPGSAGSEPATSGTEPSGT | |
| STEPSEPGSAGSEPATSGTEPSGTSTEPSEPGSAGTSTEPSEPGSAGTSTE | |
| PSEPGSAGTSTEPSEPGSAGTSTEPSEPGSAGTSTEPSEPGSAGTSEPSTS | |
| EPGAGSGASEPTSTEPGTSTEPSEPGSAGTSTEPSEPGSAGTSTEPSEPG | |
| SAGSEPATSGTEPSGSGASEPTSTEPGSEPATSGTEPSGSEPATSGTEPS | |
| GSEPATSGTEPSGSEPATSGTEPSGTSEPSTSEPGAGSEPATSGTEPSGS | |
| GASEPTSTEPGTSTEPSEPGSAGSEPATSGTEPSGSGASEPTSTEPGTSTE | |
| PSEPGSA | |
| GTSTEPSEGSAPGSEPATSGSETPGSPAGSPTSTEEGSTSSTAESPGPGTS | 63 |
| TPESGSASPGSTSESPSGTAPGSTSESPSGTAPGTSTPESGSASPGTSTPE | |
| SGSASPGSEPATSGSETPGTSESATPESGPGSPAGSPTSTEEGTSTEPSEG | |
| SAPGTSESATPESGPGTSTEPSEGSAPGTSTEPSEGSAPGSPAGSPTSTEE | |
| GTSTEPSEGSAPGTSTEPSEGSAPGTSESATPESGPGTSESATPESGPGT | |
| STEPSEGSAPGTSTEPSEGSAPGTSESATPESGPGTSTEPSEGSAPGSEPA | |
| TSGSETPGSPAGSPTSTEEGSSTPSGATGSPGTPGSGTASSSPGSSTPSG | |
| ATGSPGTSTEPSEGSAPGTSTEPSEGSAPGSEPATSGSETPGSPAGSPTS | |
| TEEGSPAGSPTSTEEGTSTEPSEGSAPGPEPTGPAPSGGSEPATSGSETP | |
| GTSESATPESGPGSPAGSPTSTEEGTSESATPESGPGSPAGSPTSTEEGSP | |
| AGSPTSTEEGTSESATPESGPGSPAGSPTSTEEGSPAGSPTSTEEGSTSST | |
| AESPGPGSTSESPSGTAPGTSPSGESSTAPGSTSESPSGTAPGSTSESPSG | |
| TAPGTSPSGESSTAPGTSTEPSEGSAPGTSESATPESGPGTSESATPESGP | |
| GSEPATSGSETPGTSESATPESGPGTSESATPESGPGTSTEPSEGSAPGT | |
| SESATPESGPGTSTEPSEGSAPGTSPSGESSTAPGTSPSGESSTAPGTSPS | |
| GESSTAPGTSTEPSEGSAPGSPAGSPTSTEEGTSTEPSEGSAPGSSPSAST | |
| GTGPGSSTPSGATGSPGSSTPSGATGSPGSSTPSGATGSPGSSTPSGATG | |
| SPGASPGTSSTGSPGASASGAPSTGGTSPSGESSTAPGSTSSTAESPGPG | |
| TSPSGESSTAPGTSESATPESGPGTSTEPSEGSAPGTSTEPSEGSAPGSSP | |
| SASTGTGPGSSTPSGATGSPGASPGTSSTGSPGTSTPESGSASPGTSPSG | |
| ESSTAPGTSPSGESSTAPGTSESATPESGPGSEPATSGSETPGTSTEPSEG | |
| SAPGSTSESPSGTAPGSTSESPSGTAPGTSTPESGSASPGSPAGSPTSTEE | |
| GTSESATPESGPGTSTEPSEGSAPGSPAGSPTSTEEGTSESATPESGPGS | |
| EPATSGSETPGSSTPSGATGSPGASPGTSSTGSPGSSTPSGATGSPGSTS | |
| ESPSGTAPGTSPSGESSTAPGSTSSTAESPGPGSSTPSGATGSPGASPGT | |
| SSTGSPGTPGSGTASSSPGSPAGSPTSTEEGSPAGSPTSTEEGTSTEPSEG | |
| SAP | |
| MAEPAGSPTSTEEGASPGTSSTGSPGSSTPSGATGSPGSSTPSGATGSPG | 64 |
| TSTEPSEGSAPGSEPATSGSETPGSPAGSPTSTEEGSTSSTAESPGPGTST | |
| PESGSASPGSTSESPSGTAPGSTSESPSGTAPGTSTPESGSASPGTSTPES | |
| GSASPGSEPATSGSETPGTSESATPESGPGSPAGSPTSTEEGTSTEPSEGS | |
| APGTSESATPESGPGTSTEPSEGSAPGTSTEPSEGSAPGSPAGSPTSTEE | |
| GTSTEPSEGSAPGTSTEPSEGSAPGTSESATPESGPGTSESATPESGPGT | |
| STEPSEGSAPGTSTEPSEGSAPGTSESATPESGPGTSTEPSEGSAPGSEPA | |
| TSGSETPGSPAGSPTSTEEGSSTPSGATGSPGTPGSGTASSSPGSSTPSG | |
| ATGSPGTSTEPSEGSAPGTSTEPSEGSAPGSEPATSGSETPGSPAGSPTS | |
| TEEGSPAGSPTSTEEGTSTEPSEGSAPGASASGAPSTGGTSESATPESGP | |
| GSPAGSPTSTEEGSPAGSPTSTEEGSTSSTAESPGPGSTSESPSGTAPGTS | |
| PSGESSTAPGTPGSGTASSSPGSSTPSGATGSPGSSPSASTGTGPGSEPA | |
| TSGSETPGTSESATPESGPGSEPATSGSETPGSTSSTAESPGPGSTSSTAE | |
| SPGPGTSPSGESSTAPGSEPATSGSETPGSEPATSGSETPGTSTEPSEGSA | |
| PGSTSSTAESPGPGTSTPESGSASPGSTSESPSGTAPGTSTEPSEGSAPGT | |
| STEPSEGSAPGTSTEPSEGSAPGSSTPSGATGSPGSSPSASTGTGPGASP | |
| GTSSTGSPGSEPATSGSETPGTSESATPESGPGSPAGSPTSTEEGSSTPSG | |
| ATGSPGSSPSASTGTGPGASPGTSSTGSPGTSESATPESGPGTSTEPSEG | |
| SAPGTSTEPSEGSAP | |
| MAEPAGSPTSTEEGTPGSGTASSSPGSSTPSGATGSPGASPGTSSTGSPG | 65 |
| SPAGSPTSTEEGTSESATPESGPGTSTEPSEGSAPGSPAGSPTSTEEGTST | |
| EPSEGSAPGTSTEPSEGSAPGTSESATPESGPGSEPATSGSETPGSEPATS | |
| GSETPGSPAGSPTSTEEGTSESATPESGPGTSTEPSEGSAPGTSTEPSEGS | |
| APGSPAGSPTSTEEGTSTEPSEGSAPGTSTEPSEGSAPGTSESATPESGP | |
| GTSTEPSEGSAPGTSESATPESGPGSEPATSGSETPGTSTEPSEGSAPGT | |
| STEPSEGSAPGTSESATPESGPGTSESATPESGPGSPAGSPTSTEEGTSES | |
| ATPESGPGSEPATSGSETPGTSESATPESGPGTSTEPSEGSAPGTSTEPSE | |
| GSAPGTSTEPSEGSAPGTSTEPSEGSAPGTSTEPSEGSAPGTSTEPSEGS | |
| APGSPAGSPTSTEEGTSTEPSEGSAPGTSESATPESGPGSEPATSGSETP | |
| GTSESATPESGPGSEPATSGSETPGTSESATPESGPGTSTEPSEGSAPGT | |
| SESATPESGPGSPAGSPTSTEEGSPAGSPTSTEEGSPAGSPTSTEEGTSES | |
| ATPESGPGTSTEPSEGSAPGTSESATPESGPGSEPATSGSETPGTSESAT | |
| PESGPGSEPATSGSETPGTSESATPESGPGTSTEPSEGSAPGSPAGSPTST | |
| EEGTSESATPESGPGSEPATSGSETPGTSESATPESGPGSPAGSPTSTEE | |
| GSPAGSPTSTEEGTSTEPSEGSAPGTSESATPESGPGTSESATPESGPGT | |
| SESATPESGPGSEPATSGSETPGSEPATSGSETPGSPAGSPTSTEEGTSTE | |
| PSEGSAPGTSTEPSEGSAPGSEPATSGSETPGTSESATPESGPGTSTEPSE | |
| GSAP | |
| GASPGTSSTGSPGSSPSASTGTGPGSSPSASTGTGPGTPGSGTASSSPGS | 66 |
| STPSGATGSPGSSPSASTGTGPGASPGTSSTGSPGTPGSGTASSSPGSST | |
| PSGATGSPGTPGSGTASSSPGASPGTSSTGSPGASPGTSSTGSPGTPGSG | |
| TASSSPGSSTPSGATGSPGASPGTSSTGSPGTPGSGTASSSPGSSTPSGA | |
| TGSPGSSPSASTGTGPGSSPSASTGTGPGSSTPSGATGSPGSSTPSGATG | |
| SPGASPGTSSTGSPGASPGTSSTGSPGASPGTSSTGSPGTPGSGTASSSP | |
| GASPGTSSTGSPGASPGTSSTGSPGASPGTSSTGSPGSSPSASTGTGPGT | |
| PGSGTASSSPGASPGTSSTGSPGASPGTSSTGSPGASPGTSSTGSPGSST | |
| PSGATGSPGSSTPSGATGSPGASPGTSSTGSPGTPGSGTASSSPGSSTPS | |
| GATGSPGSSTPSGATGSPGSSTPSGATGSPGSSPSASTGTGPGASPGTSS | |
| TGSPGASPGTSSTGSPGTPGSGTASSSPGASPGTSSTGSPGASPGTSSTG | |
| SPGASPGTSSTGSPGASPGTSSTGSPGTPGSGTASSSPGSSTPSGATGSP | |
| GTPGSGTASSSPGSSTPSGATGSPGTPGSGTASSSPGSSTPSGATGSPGS | |
| STPSGATGSPGSSPSASTGTGPGSSPSASTGTGPGASPGTSSTGSPGTPG | |
| SGTASSSPGSSTPSGATGSPGSSPSASTGTGPGSSPSASTGTGPGASPGT | |
| SSTGSPGASPGTSSTGSPGSSTPSGATGSPGSSPSASTGTGPGASPGTSS | |
| TGSPGSSPSASTGTGPGTPGSGTASSSPGSSTPSGATGSPGSSTPSGATG | |
| SPGASPGTSSTGSP | |
| GSTSESPSGTAPGTSPSGESSTAPGSTSESPSGTAPGSTSESPSGTAPGTS | 67 |
| TPESGSASPGTSTPESGSASPGSTSESPSGTAPGSTSESPSGTAPGTSPSG | |
| ESSTAPGSTSESPSGTAPGTSPSGESSTAPGTSPSGESSTAPGSTSSTAES | |
| PGPGTSPSGESSTAPGTSPSGESSTAPGSTSSTAESPGPGTSTPESGSASP | |
| GTSTPESGSASPGSTSESPSGTAPGSTSESPSGTAPGTSTPESGSASPGST | |
| SSTAESPGPGTSTPESGSASPGSTSESPSGTAPGTSPSGESSTAPGSTSST | |
| AESPGPGTSPSGESSTAPGTSTPESGSASPGSTSSTAESPGPGSTSSTAES | |
| PGPGSTSSTAESPGPGSTSSTAESPGPGTSPSGESSTAPGSTSESPSGTAP | |
| GSTSESPSGTAPGTSTPESGPXXXGASASGAPSTXXXXSESPSGTAPGS | |
| TSESPSGTAPGSTSESPSGTAPGSTSESPSGTAPGSTSESPSGTAPGSTSE | |
| SPSGTAPGTSTPESGSASPGTSPSGESSTAPGTSPSGESSTAPGSTSSTAE | |
| SPGPGTSPSGESSTAPGTSTPESGSASPGSTSESPSGTAPGSTSESPSGTA | |
| PGTSPSGESSTAPGSTSESPSGTAPGTSTPESGSASPGTSTPESGSASPGS | |
| TSESPSGTAPGTSTPESGSASPGSTSSTAESPGPGSTSESPSGTAPGSTSE | |
| SPSGTAPGTSPSGESSTAPGSTSSTAESPGPGTSPSGESSTAPGTSTPESG | |
| SASPGTSPSGESSTAPGTSPSGESSTAPGTSPSGESSTAPGSTSSTAESPG | |
| PGSTSSTAESPGPGTSPSGESSTAPGSSPSASTGTGPGSSTPSGATGSPGS | |
| STPSGATGSP | |
| GSSESGSSEGGPGSSESGSSEGGPGESPGGSSGSESGSGGEPSESGSSGE | 68 |
| SPGGSSGSESGESPGGSSGSESGSSESGSSEGGPGSSESGSSEGGPGSSE | |
| SGSSEGGPGESPGGSSGSESGESPGGSSGSESGESPGGSSGSESGSSESG | |
| SSEGGPGSSESGSSEGGPGSSESGSSEGGPGSSESGSSEGGPGSSESGSS | |
| EGGPGSSESGSSEGGPGSGGEPSESGSSGESPGGSSGSESGESPGGSSGS | |
| ESGSGGEPSESGSSGSEGSSGPGESSGSSESGSSEGGPGSGGEPSESGSS | |
| GSEGSSGPGESSGSSESGSSEGGPGSGGEPSESGSSGESPGGSSGSESGS | |
| GGEPSESGSSGSGGEPSESGSSGSSESGSSEGGPGSGGEPSESGSSGSGG | |
| EPSESGSSGSEGSSGPGESSGESPGGSSGSESGSEGSSGPGESSGSEGSS | |
| GPGESSGSGGEPSESGSSGSSESGSSEGGPGSSESGSSEGGPGESPGGSS | |
| GSESGSGGEPSESGSSGSEGSSGPGESSGESPGGSSGSESGSEGSSGPGS | |
| SESGSSEGGPGSGGEPSESGSSGSEGSSGPGESSGSEGSSGPGESSGSEG | |
| SSGPGESSGSGGEPSESGSSGSGGEPSESGSSGESPGGSSGSESGESPGG | |
| SSGSESGSGGEPSESGSSGSEGSSGPGESSGESPGGSSGSESGSSESGSSE | |
| GGPGSSESGSSEGGPGSSESGSSEGGPGSGGEPSESGSSGSSESGSSEGG | |
| PGESPGGSSGSESGSGGEPSESGSSGSSESGSSEGGPGESPGGSSGSESG | |
| SGGEPSESGSSGESPGGSSGSESGSGGEPSESGSS | |
| MAEPAGSPTSTEEGTPGSGTASSSPGSS1TSGATGSPGASPGTSSTGSPG | 69 |
| SPAGSPTSTEEGTSESATPESGPGTSTEPSEGSAPGSPAGSPTSTEEGTST | |
| EPSEGSAPGTSTEPSEGSAPOSESATPESGPGSEPATSGSETPGSEPATS | |
| GSETPGSPAGSPTSTEEGTSESATPESGPGTSTEPSEGSAPGTSTEPSEGS | |
| APGSPAGSPTSTEEGTSTEPSEGSAPGTSTEPSEGSAPGTSESATPESGP | |
| GTSTEPSEGSAPGTSESATPESGPGSEPATSGSETPGTSTEPSEGSAPGT | |
| STEPSEGSAPGSESATPESGPGTSESATPESGPGSPAGSPTSTEEGTSESA | |
| TPESGPGSEPATSGSETPGTSESATPESGPGTSTEPSEGSAPGTSTEPSEG | |
| SAPGTSTEPSEGSAPGTSTEPSEGSAPGTSTEPSEGSAPGTSTEPSEGSAP | |
| GSPAGSPTSTEEGTSTEPSEGSAPGTSESATPESGPGSEPATSGSETPGT | |
| SESATPESGPGSEPATSGSETPGTSESATPESGPGTSTEPSEGSAPGTSES | |
| ATPESGPGSPAGSPTSTEEGSPAGSPTSTEEGSPAGSPTSTEEGTSESAT | |
| PESGPGTSTEPSEGSAP | |
| ESGPGSPAGSPTSTEEGSPAGSPTSTEEGSPAGSPTSTEEGTSESATPES | 70 |
| GPGTSTEPSEGSAPGSTSSTAESPGPGSTSSTAESPGPGSTSESPSGTAPG | |
| STSSTAESPGPGSTSSTAESPGPGTSTPESGSASPGSTSESPSGTAPGTSP | |
| SGESSTAPGSTSESPSGTAPGSTSESPSGTAPGTSPSGESSTAPGSTSESP | |
| SGTAPGSTSESPSGTAPGTSPSGESSTAPGSTSESPSGTAPGSTSESPSGT | |
| APGSTSESPSGTAPGTSTPESGSASPGSTSESPSGTAPGTSTPESGSASPG | |
| STSSTAESPGPGSTSSTAESPGPGTSTPESGSASPGTSTPESGSASPGSTS | |
| ESPSGTAPGTSTPESGSASPGTSTPESGSASPGSTSESPSGTAPGSTSESP | |
| SGTAPGSTSESPSGTAPGSTSSTAESPGPGTSTPESGSASPGTSTPESGSA | |
| SPGSTSESPSGTAPGSTSESPSGTAPGTSTPESGSASPGSTSESPSGTAPG | |
| STSESPSGTAPGTSTPESGSASPGTSPSGESSTAPGSTSSTAESPGPGTSP | |
| SGESSTAPGSTSSTAESPGPGTSTPESGSASPGSTSESPSGTAPGSTSSTA | |
| ESPGPGTSTPESGSASPGTSTPESGSASP | |
| GSPAGSPTSTEEGTSESATPESGPGTSTEPSEGSAPGSPAGSPTSTEEGT | 71 |
| STEPSEGSAPGTSTEPSEGSAPGTSESATPESGPGSEPATSGSETPGSEPA | |
| TSGSETPGSPAGSPTSTEEGTSESATPESGPGTSTEPSEGSAPGTSTEPSE | |
| GSAPGSPAGSPTSTEEGTSTEPSEGSAPGTSTEPSEGSAPGTSESATPES | |
| GPGTSTEPSEGSAPGTSESATPESGPGSEPATSGSETPOSTEPSEGSAPG | |
| TSTEPSEGSAPGTSESATPESGPGTSESATPESGPGSPAGSPTSTEEGTSE | |
| SATPESGPGSEPATSGSETPGTSESATPESGPGTSTEPSEGSAPGTSTEPS | |
| EGSAPGTSTEPSEGSAPGTSTEPSEGSAPGTSTEPSEGSAPGTSTEPSEG | |
| SAPGSPAGSPTSTEEGTSTEPSEGSAPGTSESATPESGPGSEPATSGSETP | |
| GTSESATPESGPGSEPATSGSETPUSESATPESGPOSTEPSEGSAPGTSES | |
| ATP | |
| GSSESGSSEGGPGSGGEPSESGSSGSSESGSSEGGPGSSESGSSEGGPGS | 72 |
| SESGSSEGGPGSSESGSSEGGPGSSESGSSEGGPGESPGGSSGSESGSEG | |
| SSGPGESSGSSESGSSEGGPGSSESGSSEGGPGSSESGSSEGGPGSGGEP | |
| SESGSSGESPGGSSGSESGESPGGSSGSESGSGGEPSESGSSGSSESGSSE | |
| GGPGSGGEPSESGSSGSGGEPSESGSSGSEGSSGPGESSGESPGGSSGSE | |
| SGSGGEPSESGSSGSGGEPSESGSSGSGGEPSESGSSGSSESGSSEGGPG | |
| ESPGGSSGSESGESPGGSSGSESGESPGGSSGSESGESPGGSSGSESGES | |
| PGGSSGSESGSSESGSSEGGPGSGGEPSESGSSGSEGSSGPGESSGSSES | |
| GSSEGGPGSGGEPSESGSSGSSESGSSEGGPGSGGEPSESGSSGESPGGS | |
| SGSESGESPGGSSGSESGSSESGSSEGGPGSGGEPSESGSSGSSESGSSE | |
| GGPGSGGEPSESGSSGSGGEPSESGSSGESPGGSSGSESGSEGSSGPGES | |
| SGSSESGSSEGGPGSEGSSGPGESS | |
| GSTSSTAESPGPGSTSSTAESPGPGSTSESPSGTAPGSTSSTAESPGPGST | 73 |
| SSTAESPGPGTSTPESGSASPGSTSESPSGTAPGTSPSGESSTAPGSTSES | |
| PSGTAPGSTSESPSGTAPGTSPSGESSTAPGSTSESPSGTAPGSTSESPSG | |
| TAPGTSPSGESSTAPGSTSESPSGTAPGSTSESPSGTAPGSTSESPSGTAP | |
| GTSTPESGSASPGSTSESPSGTAPGTSTPESGSASPGSTSSTAESPGPGST | |
| SSTAESPGPGTSTPESGSASPGTSTPESGSASPGSTSESPSGTAPGTSTPE | |
| SGSASPGTSTPESGSASPGSTSESPSGTAPGSTSESPSGTAPGSTSESPSG | |
| TAPGSTSSTAESPGPGTSTPESGSASPGTSTPESGSASPGSTSESPSGTAP | |
| GSTSESPSGTAPGTSTPESGSASPGSTSESPSGTAPGSTSESPSGTAPGTS | |
| TPESGSASPGTSPSGESSTAPGSTSSTAESPGPGTSPSGESSTAPGSTSST | |
| AESPGPGTSTPESGSASPGSTSESPSGTAP | |
| GASPGTSSTGSPGSSPSASTGTGPGSSPSASTGTGPGTPGSGTASSSPGS | 74 |
| STPSGATGSPGSXPSASTGTGPGASPGTSSTGSPGTPGSGTASSSPGSST | |
| PSGATGSPGTPGSGTASSSPGASPGTSSTGSPGASPGTSSTGSPGTPGSG | |
| TASSSPGSSTPSGATGSPGASPGTSSTGSPGTPGSGTASSSPGSSTPSGA | |
| TGSPGSXPSASTGTGPGSSPSASTGTGPGSSTPSGATGSPGSSTPSGATG | |
| SPGASPGTSSTGSPGASPGTSSTGSPGASPGTSSTGSPGTPGSGTASSSP | |
| GASPGTSSTGSPGASPGTSSTGSPGASPGTSSTGSPGSSPSASTGTGPGT | |
| PGSGTASSSPGASPGTSSTGSPGASPGTSSTGSPGASPGTSSTGSPGSST | |
| PSGATGSPGSSTPSGATGSPGASPGTSSTGSPGTPGSGTASSSPGSSTPS | |
| GATGSPGSSTPSGATGSPGSSTPSGATGSPGSSPSASTGTGPGASPGTSS | |
| TGSP | |

TABLE 3
Antagonists of GLP-1 Receptor Fusion Proteins.

| Sequences | Seq ID No. |
|---|---|
| DLSKQMEEEAVRLFIEWLKNGGPSSGAPPPSGSPAGSPTSTEEGTSESA | 19 |
| TPESGPGTSTEPSEGSAPGSPAGSPTSTEEGTSTEPSEGSAPGTSTEPSEG | |
| SAPGTSESATPESGPGSEPATSGSETPGSEPATSGSETPGSPAGSPTSTEE | |
| GTSESATPESGPGTSTEPSEGSAPGTSTEPSEGSAPGSPAGSPTSTEEGT | |
| STEPSEGSAPGTSTEPSEGSAPGTSESATPESGPGTSTEPSEGSAPGTSES | |
| ATPESGPGSEPATSGSETPGTSTEPSEGSAPGTSTEPSEGSAPGTSESAT | |
| PESGPGTSESATPESGPGSPAGSPTSTEEGTSESATPESGPGSEPATSGSE | |
| TPGTSESATPESGPGTSTEPSEGSAPGTSTEPSEGSAPGTSTEPSEGSAP | |
| GTSTEPSEGSAPGTSTEPSEGSAPGTSTEPSEGSAPGSPAGSPTSTEEGT | |
| STEPSEGSAPGTSESATPESGPGSEPATSGSETPGTSESATPESGPGSEPA | |
| TSGSETPGTSESATPESGPGTSTEPSEGSAPGTSESATPESGPGSPAGSPT | |
| STEEGSPAGSPTSTEEGSPAGSPTSTEEGTSESATPESGPGTSTEPSEGSA | |
| PGTSESATPESGPGSEPATSGSETPGTSESATPESGPGSEPATSGSETPG | |
| TSESATPESGPGTSTEPSEGSAPGSPAGSPTSTEEGTSESATPESGPGSEP | |
| ATSGSETPGTSESATPESGPGSPAGSPTSTEEGSPAGSPTSTEEGTSTEPS | |
| EGSAPGTSESATPESGPGTSESATPESGPGTSESATPESGPGSEPATSGS | |
| ETPGSEPATSGSETPGSPAGSPTSTEEGTSTEPSEGSAP | |
| DLSKQMEEEAVRLFIEWLKNGGPSSGAPPPSGTSTEPSEGSAPGSEPAT | 20 |
| SGSETPGSPAGSPTSTEEGSTSSTAESPGPGTSTPESGSASPGSTSESPSG | |
| TAPGSTSESPSGTAPGTSTPESGSASPGTSTPESGSASPGSEPATSGSETP | |
| GTSESATPESGPGSPAGSPTSTEEGTSTEPSEGSAPGTSESATPESGPGT | |
| STEPSEGSAPGTSTEPSEGSAPGSPAGSPTSTEEGTSTEPSEGSAPGTSTE | |
| PSEGSAPGTSESATPESGPGTSESATPESGPGTSTEPSEGSAPGTSTEPSE | |
| GSAPGTSESATPESGPGTSTEPSEGSAPGSEPATSGSETPGSPAGSPTST | |
| EEGSSTPSGATGSPGTPGSGTASSSPGSSTPSGATGSPGTSTEPSEGSAP | |
| GTSTEPSEGSAPGSEPATSGSETPGSPAGSPTSTEEGSPAGSPTSTEEGT | |
| STEPSEGSAPGASASGAPSTGGTSESATPESGPGSPAGSPTSTEEGSPAG | |
| SPTSTEEGSTSSTAESPGPGSTSESPSGTAPGTSPSGESSTAPGTPGSGTA | |
| SSSPGSSTPSGATGSPGSSPSASTGTGPGSEPATSGSETPGTSESATPESG | |
| PGSEPATSGSETPGSTSSTAESPGPGSTSSTAESPGPGTSPSGESSTAPGS | |
| EPATSGSETPGSEPATSGSETPGTSTEPSEGSAPGSTSSTAESPGPGTSTP | |
| ESGSASPGSTSESPSGTAPGTSTEPSEGSAPGTSTEPSEGSAPGTSTEPSE | |
| GSAPGSSTPSGATGSPGSSPSASTGTGPGASPGTSSTGSPGSEPATSGSE | |
| TPGTSESATPESGPGGSPAGSPTSTEEGSSTPSGATGSPGSSPSASTGTG | |
| PGASPGTSSTSPGTSESATPESGPGTSTEPSEGSAPGTSTEPSEGSAP | |
| DLSKQMEEEAVRLFIEWLKNGGPSSGAPPPSVPGVGVPGVGVPGGGV | |
| PGAGVPGVGVPGVGVPGVGVPGGGVPGAGVPGGGVPGVGVPGVGV | 21 |
| PGGGVPGAGVPGVGVPGVGVPGVGVPGGGVPGAGVPGGGVPGVGV | |
| PGVGVPGGGVPGAGVPGVGVPGVGVPGVGVPGGGVPGAGVPGGGV | |
| PGVGVPGVGVPGGGVPGAGVPGVGVPGVGVPGVGVPGGGVPGAGV | |
| PGGGVPGVGVPGVGVPGGGVPGAGVPGVGVPGVGVPGVGVPGGGV | |
| PGAGVPGGGVPGVGVPGVGVPGGGVPGAGVPGVGVPGVGVPGVGV | |
| PGGGVPGAGVPGGGVPGVGVPGVGVPGGGVPGAGVPGVGVPGVGV | |
| PGVGVPGGGVPGAGVPGGGVPGVGVPGVGVPGGGVPGAGVPGVGV | |
| PGVGVPGVGVPGGGVPGAGVPGGGVPGVGVPGVGVPGGGVPGAGV | |
| PGVGVPGVGVPGVGVPGGGVPGAGVPGGGVPGVGVPGVGVPGGGV | |
| PGAGVPGVGVPGVGVPGVGVPGGGVPGAGVPGGGVPGVGVPGVGV | |
| PGGGVPGAGVPGVGVPGVGVPGVGVPGGGVPGAGVPGGGVPGVGV | |
| PGVGVPGGGVPGAGVPGVGVPGVGVPGVGVPGGGVPGAGVPGGGV | |
| PGWP | |
| HAEGTFTSDVSSYLEGQAAKEFIAAWLVKGRGSPAGSPTSTEEGTSES | 22 |
| ATPESGPGTSTEPSEGSAPGSPAGSPTSTEEGTSTEPSEGSAPGTSTEPSE | |
| GSAPGTSESATPESGPGSEPATSGSETPGSEPATSGSETPGSPAGSPTST | |
| EEGTSESATPESGPGTSTEPSEGSAPGTSTEPSEGSAPGSPAGSPTSTEE | |
| GTSTEPSEGSAPGTSTEPSEGSAPGTSESATPESGPGTSTEPSEGSAPGT | |
| SESATPESGPGSEPATSGSETPGTSTEPSEGSAPGTSTEPSEGSAPGTSES | |
| ATPESGPGTSESATPESGPGSPAGSPTSTEEGTSESATPESGPGSEPATS | |
| GSETPGTSESATPESGPGTSTEPSEGSAPGTSTEPSEGSAPGTSTEPSEGS | |
| APGTSTEPSEGSAPGTSTEPSEGSAPGTSTEPSEGSAPGSPAGSPTSTEE | |
| GTSTEPSEGSAPGTSESATPESGPGSEPATSGSETPGTSESATPESGPGS | |
| EPATSGSETPGTSESATPESGPGTSTEPSEGSAPGTSESATPESGPGSPA | |
| GSPTSTEEGSPAGSPTSTEEGSPAGSPTSTEEGTSESATPESGPGTSTEPS | |
| EGSAPGTSESATPESGPGSEPATSGSETPGTSESATPESGPGSEPATSGS | |
| ETPGTSESATPESGPGTSTEPSEGSAPGSPAGSPTSTEEGTSESATPESGP | |
| GSEPATSGSETPGTSESATPESGPGSPAGSPTSTEEGSPAGSPTSTEEGT | |
| STEPSEGSAPGTSESATPESGPGTSESATPESGPGTSESATPESGPGSEPA | |
| TSGSETPGSEPATSGSETPGSPAGSPTSTEEGTSTEPSEGSAP | |
| HAEGTFTSDVSSYLEGQAAKEFIAAWLVKGRGTSTEPSEGSAPGSEPA | 23 |
| TSGSETPGSPAGSPTSTEEGSTSSTAESPGPGTSTPESGSASPGSTSESPS | |
| GTAPGSTSESPSGTAPGTSTPESGSASPGTSTPESGSASPGSEPATSGSE | |
| TPGTSESATPESGPGSPAGSPTSTEEGTSTEPSEGSAPGTSESATPESGP | |
| GTSTEPSEGSAPGTSTEPSEGSAPGSPAGSPTSTEEGTSTEPSEGSAPGT | |
| STEPSEGSAPGTSESATPESGPGTSESATPESGPGTSTEPSEGSAPGTSTE | |
| PSEGSAPGTSESATPESGPGTSTEPSEGSAPGSEPATSGSETPGSPAGSP | |
| TSTEEGSSTPSGATGSPGTPGSGTASSSPGSSTPSGATGSPGTSTEPSEG | |
| SAPGTSTEPSEGSAPGSEPATSGSETPGSPAGSPTSTEEGSPAGSPTSTEE | |
| GTSTEPSEGSAPGASASGAPSTGGTSESATPESGPGSPAGSPTSTEEGSP | |
| AGSPTSTEEGSTSSTAESPGPGSTSESPSGTAPGTSPSGESSTAPGTPGS | |
| GTASSSPGSSTPSGATGSPGSSPSASTGTGPGSEPATSGSETPGTSESAT | |
| PESGPGSEPATSGSETPGSTSSTAESPGPGSTSSTAESPGPGTSPSGESST | |
| APGSEPATSGSETPGSEPATSGSETPGTSTEPSEGSAPGSTSSTAESPGP | |
| GTSTPESGSASPGSTSESPSGTAPGTSTEPSEGSAPGTSTEPSEGSAPGTS | |
| TEPSEGSAPGSSTPSGATGSPGSSPSASTGTGPGASPGTSSTGSPGSEPA | |
| TSGSETPGTSESATPESGPGGSPAGSPTSTEEGSSTPSGATGSPGSSPSAS | |
| TGTGPGASPGTSSTSPGTSESATPESGPGTSTEPSEGSAPGTSTEPSEGS | |
| AP | |
| HAEGTFTSDVSSYLEGQAAKEFIAAWLVKGRVPGVGVPGVGVPGGG | 24 |
| VPGAGVPGVGVPGVGVPGVGVPGGGVPGAGVPGGGVPGVGVPGVG | |
| VPGGGVPGAGVPGVGVPGVGVPGVGVPGGGVPGAGVPGGGVPGVG | |
| VPGVGVPGGGVPGAGVPGVGVPGVGVPGVGVPGGGVPGAGVPGGG | |
| VPGVGVPGVGVPGGGVPGAGVPGVGVPGVGVPGVGVPGGGVPGAG | |
| VPGGGVPGVGVPGVGVPGGGVPGAGVPGVGVPGVGVPGVGVPGGG | |
| VPGAGVPGGGVPGVGVPGVGVPGGGVPGAGVPGVGVPGVGVPGVG | |
| VPGGGVPGAGVPGGGVPGVGVPGVGVPGGGVPGAGVPGVGVPGVG | |
| VPGVGVPGGGVPGAGVPGGGVPGVGVPGVGVPGGGVPGAGVPGVG | |
| VPGVGVPGVGVPGGGVPGAGVPGGGVPGVGVPGVGVPGGGVPGAG | |
| VPGVGVPGVGVPGVGVPGGGVPGAGVPGGGVPGVGVPGVGVPGGG | |
| VPGAGVPGVGVPGVGVPGVGVPGGGVPGAGVPGGGVPGVGVPGVG | |
| VPGGGVPGAGVPGVGVPGVGVPGVGVPGGGVPGAGVPGGGVPGVG | |
| VPGVGVPGGGVPGAGVPGVGVPGVGVPGVGVPGGGVPGAGVPGGG | |
| VPGWP | |
| HGEGTFTSDLSKQMEEEAVRLFIEWLKNGGPSSGAPPPSGSPAGSPTST | 25 |
| EEGTSESATPESGPGTSTEPSEGSAPGSPAGSPTSTEEGTSTEPSEGSAP | |
| GTSTEPSEGSAPGTSESATPESGPGSEPATSGSETPGSEPATSGSETPGSP | |
| AGSPTSTEEGTSESATPESGPGTSTEPSEGSAPGTSTEPSEGSAPGSPAG | |
| SPTSTEEGTSTEPSEGSAPGTSTEPSEGSAPGTSESATPESGPGTSTEPSE | |
| GSAPGTSESATPESGPGSEPATSGSETPGTSTEPSEGSAPGTSTEPSEGS | |
| APGTSESATPESGPGTSESATPESGPGSPAGSPTSTEEGTSESATPESGP | |
| GSEPATSGSETPGTSESATPESGPGTSTEPSEGSAPGTSTEPSEGSAPGT | |
| STEPSEGSAPGTSTEPSEGSAPGTSTEPSEGSAPGTSTEPSEGSAPGSPA | |
| GSPTSTEEGTSTEPSEGSAPGTSESATPESGPGSEPATSGSETPGTSESAT | |
| PESGPGSEPATSGSETPGTSESATPESGPGTSTEPSEGSAPGTSESATPES | |
| GPGSPAGSPTSTEEGSPAGSPTSTEEGSPAGSPTSTEEGTSESATPESGP | |
| GTSTEPSEGSAPGTSESATPESGPGSEPATSGSETPGTSESATPESGPGS | |
| EPATSGSETPGTSESATPESGPGTSTEPSEGSAPGSPAGSPTSTEEGTSES | |
| ATPESGPGSEPATSGSETPGTSESATPESGPGSPAGSPTSTEEGSPAGSP | |
| TSTEEGTSTEPSEGSAPGTSESATPESGPGTSESATPESGPGTSESATPES | |
| GPGSEPATSGSETPGSEPATSGSETPGSPAGSPTSTEEGTSTEPSEGSAP | |
| HGEGTFTSDLSKQMEEEAVRLFIEWLKNGGPSSGAPPPSGTSTEPSEGS | 26 |
| APGSEPATSGSETPGSPAGSPTSTEEGSTSSTAESPGPGTSTPESGSASP | |
| GSTSESPSGTAPGSTSESPSGTAPGTSTPESGSASPGTSTPESGSASPGSE | |
| PATSGSETPGTSESATPESGPGSPAGSPTSTEEGTSTEPSEGSAPGTSES | |
| ATPESGPGTSTEPSEGSAPGTSTEPSEGSAPGSPAGSPTSTEEGTSTEPSE | |
| GSAPGTSTEPSEGSAPGTSESATPESGPGTSESATPESGPGTSTEPSEGS | |
| APGTSTEPSEGSAPGTSESATPESGPGTSTEPSEGSAPGSEPATSGSETP | |
| GSPAGSPTSTEEGSSTPSGATGSPGTPGSGTASSSPGSSTPSGATGSPGT | |
| STEPSEGSAPGTSTEPSEGSAPGSEPATSGSETPGSPAGSPTSTEEGSPA | |
| GSPTSTEEGTSTEPSEGSAPGASASGAPSTGGTSESATPESGPGSPAGSP | |
| TSTEEGSPAGSPTSTEEGSTSSTAESPGPGSTSESPSGTAPGTSPSGESST | |
| APGTPGSGTASSSPGSSTPSGATGSPGSSPSASTGTGPGSEPATSGSETP | |
| GTSESATPESGPGSEPATSGSETPGSTSSTAESPGPGSTSSTAESPGPGTS | |
| PSGESSTAPGSEPATSGSETPGSEPATSGSETPGTSTEPSEGSAPGSTSST | |
| AESPGPGTSTPESGSASPGSTSESPSGTAPGTSTEPSEGSAPGTSTEPSEG | |
| SAPGTSTEPSEGSAPGSSTPSGATGSPGSSPSASTGTGPGASPGTSSTGS | |
| PGSEPATSGSETPGTSESATPESGPGGSPAGSPTSTEEGSSTPSGATGSP | |
| GSSPSASTGTGPGASPGTSSTSPGTSESATPESGPGTSTEPSEGSAPGTS | |
| TEPSEGSAP | |
| HGEGTFTSDLSKQMEEEAVRLFIEWLKNGGPSSGAPPPSVPGVGVPGV | 27 |
| GVPGGGVPGAGVPGVGVPGVGVPGVGVPGGGVPGAGVPGGGVPGV | |
| GVPGVGVPGGGVPGAGVPGVGVPGVGVPGVGVPGGGVPGAGVPGG | |
| GVPGVGVPGVGVPGGGVPGAGVPGVGVPGVGVPGVGVPGGGVPGA | |
| GVPGGGVPGVGVPGVGVPGGGVPGAGVPGVGVPGVGVPGVGVPGG | |
| GVPGAGVPGGGVPGVGVPGVGVPGGGVPGAGVPGVGVPGVGVPGV | |
| GVPGGGVPGAGVPGGGVPGVGVPGVGVPGGGVPGAGVPGVGVPGV | |
| GVPGVGVPGGGVPGAGVPGGGVPGVGVPGVGVPGGGVPGAGVPGV | |
| GVPGVGVPGVGVPGGGVPGAGVPGGGVPGVGVPGVGVPGGGVPGA | |
| GVPGVGVPGVGVPGVGVPGGGVPGAGVPGGGVPGVGVPGVGVPGG | |
| GVPGAGVPGVGVPGVGVPGVGVPGGGVPGAGVPGGGVPGVGVPGV | |
| GVPGGGVPGAGVPGVGVPGVGVPGVGVPGGGVPGAGVPGGGVPGV | |
| GVPGVGVPGGGVPGAGVPGVGVPGVGVPGVGVPGGGVPGAGVPGG | |
| GVPGVGVPGVGVPGGGVPGAGVPGVGVPGVGVPGVGVPGGGVPGA | |
| GVPGGGVPGWP | |
| GEGTFTWELSKQMEEEAVRLFIEWLKNGGPSSGAPPPSGSPAGSPTST | 28 |
| EEGTSESATPESGPGTSTEPSEGSAPGSPAGSPTSTEEGTSTEPSEGSAP | |
| GTSTEPSEGSAPGTSESATPESGPGSEPATSGSETPGSEPATSGSETPGSP | |
| AGSPTSTEEGTSESATPESGPGTSTEPSEGSAPGTSTEPSEGSAPGSPAG | |
| SPTSTEEGTSTEPSEGSAPGTSTEPSEGSAPGTSESATPESGPGTSTEPSE | |
| GSAPGTSESATPESGPGSEPATSGSETPGTSTEPSEGSAPGTSTEPSEGS | |
| APGTSESATPESGPGTSESATPESGPGSPAGSPTSTEEGTSESATPESGP | |
| GSEPATSGSETPGTSESATPESGPGTSTEPSEGSAPGTSTEPSEGSAPGT | |
| STEPSEGSAPGTSTEPSEGSAPGTSTEPSEGSAPGTSTEPSEGSAPGSPA | |
| GSPTSTEEGTSTEPSEGSAPGTSESATPESGPGSEPATSGSETPGTSESAT | |
| PESGPGSEPATSGSETPGTSESATPESGPGTSTEPSEGSAPGTSESATPES | |
| GPGSPAGSPTSTEEGSPAGSPTSTEEGSPAGSPTSTEEGTSESATPESGP | |
| GTSTEPSEGSAPGTSESATPESGPGSEPATSGSETPGTSESATPESGPGS | |
| EPATSGSETPGTSESATPESGPGTSTEPSEGSAPGSPAGSPTSTEEGTSES | |
| ATPESGPGSEPATSGSETPGTSESATPESGPGSPAGSPTSTEEGSPAGSP | |
| TSTEEGTSTEPSEGSAPGTSESATPESGPGTSESATPESGPGTSESATPES | |
| GPGSEPATSGSETPGSEPATSGSETPGSPAGSPTSTEEGTSTEPSEGSAP | |
| GEGTFTWELSKQMEEEAVRLFIEWLKNGGPSSGAPPPSGTSTEPSEGS | 29 |
| APGSEPATSGSETPGSPAGSPTSTEEGSTSSTAESPGPGTSTPESGSASP | |
| GSTSESPSGTAPGSTSESPSGTAPGTSTPESGSASPGTSTPESGSASPGSE | |
| PATSGSETPGTSESATPESGPGSPAGSPTSTEEGTSTEPSEGSAPGTSES | |
| ATPESGPGTSTEPSEGSAPGTSTEPSEGSAPGSPAGSPTSTEEGTSTEPSE | |
| GSAPGTSTEPSEGSAPGTSESATPESGPGTSESATPESGPGTSTEPSEGS | |
| APGTSTEPSEGSAPGTSESATPESGPGTSTEPSEGSAPGSEPATSGSETP | |
| GSPAGSPTSTEEGSSTPSGATGSPGTPGSGTASSSPGSSTPSGATGSPGT | |
| STEPSEGSAPGTSTEPSEGSAPGSEPATSGSETPGSPAGSPTSTEEGSPA | |
| GSPTSTEEGTSTEPSEGSAPGASASGAPSTGGTSESATPESGPGSPAGSP | |
| TSTEEGSPAGSPTSTEEGSTSSTAESPGPGSTSESPSGTAPGTSPSGESST | |
| APGTPGSGTASSSPGSSTPSGATGSPGSSPSASTGTGPGSEPATSGSETP | |
| GTSESATPESGPGSEPATSGSETPGSTSSTAESPGPGSTSSTAESPGPGTS | |
| PSGESSTAPGSEPATSGSETPGSEPATSGSETPGTSTEPSEGSAPGSTSST | |
| AESPGPGTSTPESGSASPGSTSESPSGTAPGTSTEPSEGSAPGTSTEPSEG | |
| SAPGTSTEPSEGSAPGSSTPSGATGSPGSSPSASTGTGPGASPGTSSTGS | |
| PGSEPATSGSETPGTSESATPESGPGGSPAGSPTSTEEGSSTPSGATGSP | |
| GSSPSASTGTGPGASPGTSSTSPGTSESATPESGPGTSTEPSEGSAPGTS | |
| TEPSEGSAP | |
| GEGTFTWELSKQMEEEAVRLFIEWLKNGGPSSGAPPPSVPGVGVPGV | 30 |
| GVPGGGVPGAGVPGVGVPGVGVPGVGVPGGGVPGAGVPGGGVPGV | |
| GVPGVGVPGGGVPGAGVPGVGVPGVGVPGVGVPGGGVPGAGVPGG | |
| GVPGVGVPGVGVPGGGVPGAGVPGVGVPGVGVPGVGVPGGGVPGA | |
| GVPGGGVPGVGVPGVGVPGGGVPGAGVPGVGVPGVGVPGVGVPGG | |
| GVPGAGVPGGGVPGVGVPGVGVPGGGVPGAGVPGVGVPGVGVPGV | |
| GVPGGGVPGAGVPGGGVPGVGVPGVGVPGGGVPGAGVPGVGVPGV | |
| GVPGVGVPGGGVPGAGVPGGGVPGVGVPGVGVPGGGVPGAGVPGV | |
| GVPGVGVPGVGVPGGGVPGAGVPGGGVPGVGVPGVGVPGGGVPGA | |
| GVPGVGVPGVGVPGVGVPGGGVPGAGVPGGGVPGVGVPGVGVPGG | |
| GVPGAGVPGVGVPGVGVPGVGVPGGGVPGAGVPGGGVPGVGVPGV | |
| GVPGGGVPGAGVPGVGVPGVGVPGVGVPGGGVPGAGVPGGGVPGV | |
| GVPGVGVPGGGVPGAGVPGVGVPGVGVPGVGVPGGGVPGAGVPGG | |
| GVPGVGVPGVGVPGGGVPGAGVPGVGVPGVGVPGVGVPGGGVPGA | |
| GVPGGGVPGWP | |
| GEGTFTSQLSKQMEEEAVRLFIEWLKNGGPSSGAPPPSGSPAGSPTSTE | 31 |
| EGTSESATPESGPGTSTEPSEGSAPGSPAGSPTSTEEGTSTEPSEGSAPG | |
| TSTEPSEGSAPGTSESATPESGPGSEPATSGSETPGSEPATSGSETPGSPA | |
| GSPTSTEEGTSESATPESGPGTSTEPSEGSAPGTSTEPSEGSAPGSPAGSP | |
| TSTEEGTSTEPSEGSAPGTSTEPSEGSAPGTSESATPESGPGTSTEPSEGS | |
| APGTSESATPESGPGSEPATSGSETPGTSTEPSEGSAPGTSTEPSEGSAP | |
| GTSESATPESGPGTSESATPESGPGSPAGSPTSTEEGTSESATPESGPGS | |
| EPATSGSETPGTSESATPESGPGTSTEPSEGSAPGTSTEPSEGSAPGTSTE | |
| PSEGSAPGTSTEPSEGSAPGTSTEPSEGSAPGTSTEPSEGSAPGSPAGSP | |
| TSTEEGTSTEPSEGSAPGTSESATPESGPGSEPATSGSETPGTSESATPES | |
| GPGSEPATSGSETPGTSESATPESGPGTSTEPSEGSAPGTSESATPESGP | |
| GSPAGSPTSTEEGSPAGSPTSTEEGSPAGSPTSTEEGTSESATPESGPGT | |
| STEPSEGSAPGTSESATPESGPGSEPATSGSETPGTSESATPESGPGSEPA | |
| TSGSETPGTSESATPESGPGTSTEPSEGSAPGSPAGSPTSTEEGTSESATP | |
| ESGPGSEPATSGSETPGTSESATPESGPGSPAGSPTSTEEGSPAGSPTSTE | |
| EGTSTEPSEGSAPGTSESATPESGPGTSESATPESGPGTSESATPESGPG | |
| SEPATSGSETPGSEPATSGSETPGSPAGSPTSTEEGTSTEPSEGSAP | |
| GEGTFTSQLSKQMEEEAVRLFIEWLKNGGPSSGAPPPSGTSTEPSEGSA | 32 |
| PGSEPATSGSETPGSPAGSPTSTEEGSTSSTAESPGPGTSTPESGSASPGS | |
| TSESPSGTAPGSTSESPSGTAPGTSTPESGSASPGTSTPESGSASPGSEPA | |
| TSGSETPGTSESATPESGPGSPAGSPTSTEEGTSTEPSEGSAPGTSESATP | |
| ESGPGTSTEPSEGSAPGTSTEPSEGSAPGSPAGSPTSTEEGTSTEPSEGS | |
| APGTSTEPSEGSAPGTSESATPESGPGTSESATPESGPGTSTEPSEGSAP | |
| GTSTEPSEGSAPGTSESATPESGPGTSTEPSEGSAPGSEPATSGSETPGSP | |
| AGSPTSTEEGSSTPSGATGSPGTPGSGTASSSPGSSTPSGATGSPGTSTE | |
| PSEGSAPGTSTEPSEGSAPGSEPATSGSETPGSPAGSPTSTEEGSPAGSP | |
| TSTEEGTSTEPSEGSAPGASASGAPSTGGTSESATPESGPGSPAGSPTST | |
| EEGSPAGSPTSTEEGSTSSTAESPGPGSTSESPSGTAPGTSPSGESSTAPG | |
| TPGSGTASSSPGSSTPSGATGSPGSSPSASTGTGPGSEPATSGSETPGTS | |
| ESATPESGPGSEPATSGSETPGSTSSTAESPGPGSTSSTAESPGPGTSPSG | |
| ESSTAPGSEPATSGSETPGSEPATSGSETPGTSTEPSEGSAPGSTSSTAES | |
| PGPGTSTPESGSASPGSTSESPSGTAPGTSTEPSEGSAPGTSTEPSEGSAP | |
| GTSTEPSEGSAPGSSTPSGATGSPGSSPSASTGTGPGASPGTSSTGSPGS | |
| EPATSGSETPGTSESATPESGPGGSPAGSPTSTEEGSSTPSGATGSPGSS | |
| PSASTGTGPGASPGTSSTSPGTSESATPESGPGTSTEPSEGSAPGTSTEPS | |
| EGSAP | |
| GEGTFTSQLSKQMEEEAVRLFIEWLKNGGPSSGAPPPSVPGVGVPGVG | 33 |
| VPGGGVPGAGVPGVGVPGVGVPGVGVPGGGVPGAGVPGGGVPGVG | |
| VPGVGVPGGGVPGAGVPGVGVPGVGVPGVGVPGGGVPGAGVPGGG | |
| VPGVGVPGVGVPGGGVPGAGVPGVGVPGVGVPGVGVPGGGVPGAG | |
| VPGGGVPGVGVPGVGVPGGGVPGAGVPGVGVPGVGVPGVGVPGGG | |
| VPGAGVPGGGVPGVGVPGVGVPGGGVPGAGVPGVGVPGVGVPGVG | |
| VPGGGVPGAGVPGGGVPGVGVPGVGVPGGGVPGAGVPGVGVPGVG | |
| VPGVGVPGGGVPGAGVPGGGVPGVGVPGVGVPGGGVPGAGVPGVG | |
| VPGVGVPGVGVPGGGVPGAGVPGGGVPGVGVPGVGVPGGGVPGAG | |
| VPGVGVPGVGVPGVGVPGGGVPGAGVPGGGVPGVGVPGVGVPGGG | |
| VPGAGVPGVGVPGVGVPGVGVPGGGVPGAGVPGGGVPGVGVPGVG | |
| VPGGGVPGAGVPGVGVPGVGVPGVGVPGGGVPGAGVPGGGVPGVG | |
| VPGVGVPGGGVPGAGVPGVGVPGVGVPGVGVPGGGVPGAGVPGGG | |
| VPGVGVPGVGVPGGGVPGAGVPGVGVPGVGVPGVGVPGGGVPGAG | |
| VPGGGVPGWP | |
| KRHSDGTFTSDLSKQMEEEAVRLFIEWLKNGGPSSGAPPPSGSPAGSP | 34 |
| TSTEEGTSESATPESGPGTSTEPSEGSAPGSPAGSPTSTEEGTSTEPSEGS | |
| APGTSTEPSEGSAPGTSESATPESGPGSEPATSGSETPGSEPATSGSETP | |
| GSPAGSPTSTEEGTSESATPESGPGTSTEPSEGSAPGTSTEPSEGSAPGSP | |
| AGSPTSTEEGTSTEPSEGSAPGTSTEPSEGSAPGTSESATPESGPGTSTEP | |
| SEGSAPGTSESATPESGPGSEPATSGSETPGTSTEPSEGSAPGTSTEPSE | |
| GSAPGTSESATPESGPGTSESATPESGPGSPAGSPTSTEEGTSESATPES | |
| GPGSEPATSGSETPGTSESATPESGPGTSTEPSEGSAPGTSTEPSEGSAP | |
| GTSTEPSEGSAPGTSTEPSEGSAPGTSTEPSEGSAPGTSTEPSEGSAPGSP | |
| AGSPTSTEEGTSTEPSEGSAPGTSESATPESGPGSEPATSGSETPGTSES | |
| ATPESGPGSEPATSGSETPGTSESATPESGPGTSTEPSEGSAPGTSESAT | |
| PESGPGSPAGSPTSTEEGSPAGSPTSTEEGSPAGSPTSTEEGTSESATPES | |
| GPGTSTEPSEGSAPGTSESATPESGPGSEPATSGSETPGTSESATPESGP | |
| GSEPATSGSETPGTSESATPESGPGTSTEPSEGSAPGSPAGSPTSTEEGT | |
| SESATPESGPGSEPATSGSETPGTSESATPESGPGSPAGSPTSTEEGSPA | |
| GSPTSTEEGTSTEPSEGSAPGTSESATPESGPGTSESATPESGPGTSESAT | |
| PESGPGSEPATSGSETPGSEPATSGSETPGSPAGSPTSTEEGTSTEPSEGS | |
| AP | |
| KRHSDGTFTSDLSKQMEEEAVRLFIEWLKNGGPSSGAPPPSGTSTEPSE | 35 |
| GSAPGSEPATSGSETPGSPAGSPTSTEEGSTSSTAESPGPGTSTPESGSA | |
| SPGSTSESPSGTAPGSTSESPSGTAPGTSTPESGSASPGTSTPESGSASPG | |
| SEPATSGSETPGTSESATPESGPGSPAGSPTSTEEGTSTEPSEGSAPGTSE | |
| SATPESGPGTSTEPSEGSAPGTSTEPSEGSAPGSPAGSPTSTEEGTSTEPS | |
| EGSAPGTSTEPSEGSAPGTSESATPESGPGTSESATPESGPGTSTEPSEG | |
| SAPGTSTEPSEGSAPGTSESATPESGPGTSTEPSEGSAPGSEPATSGSETP | |
| GSPAGSPTSTEEGSSTPSGATGSPGTPGSGTASSSPGSSTPSGATGSPGT | |
| STEPSEGSAPGTSTEPSEGSAPGSEPATSGSETPGSPAGSPTSTEEGSPA | |
| GSPTSTEEGTSTEPSEGSAPGASASGAPSTGGTSESATPESGPGSPAGSP | |
| TSTEEGSPAGSPTSTEEGSTSSTAESPGPGSTSESPSGTAPGTSPSGESST | |
| APGTPGSGTASSSPGSSTPSGATGSPGSSPSASTGTGPGSEPATSGSETP | |
| GTSESATPESGPGSEPATSGSETPGSTSSTAESPGPGSTSSTAESPGPGTS | |
| PSGESSTAPGSEPATSGSETPGSEPATSGSETPGTSTEPSEGSAPGSTSST | |
| AESPGPGTSTPESGSASPGSTSESPSGTAPGTSTEPSEGSAPGTSTEPSEG | |
| SAPGTSTEPSEGSAPGSSTPSGATGSPGSSPSASTGTGPGASPGTSSTGS | |
| PGSEPATSGSETPGTSESATPESGPGGSPAGSPTSTEEGSSTPSGATGSP | |
| GSSPSASTGTGPGASPGTSSTSPGTSESATPESGPGTSTEPSEGSAPGTS | |
| TEPSEGSAP | |
| KRHSDGTFTSDLSKQMEEEAVRLFIEWLKNGGPSSGAPPPSVPGVGVP | 36 |
| GVGVPGGGVPGAGVPGVGVPGVGVPGVGVPGGGVPGAGVPGGGVP | |
| GVGVPGVGVPGGGVPGAGVPGVGVPGVGVPGVGVPGGGVPGAGVP | |
| GGGVPGVGVPGVGVPGGGVPGAGVPGVGVPGVGVPGVGVPGGGVP | |
| GAGVPGGGVPGVGVPGVGVPGGGVPGAGVPGVGVPGVGVPGVGVP | |
| GGGVPGAGVPGGGVPGVGVPGVGVPGGGVPGAGVPGVGVPGVGVP | |
| GVGVPGGGVPGAGVPGGGVPGVGVPGVGVPGGGVPGAGVPGVGVP | |
| GVGVPGVGVPGGGVPGAGVPGGGVPGVGVPGVGVPGGGVPGAGVP | |
| GVGVPGVGVPGVGVPGGGVPGAGVPGGGVPGVGVPGVGVPGGGVP | |
| GAGVPGVGVPGVGVPGVGVPGGGVPGAGVPGGGVPGVGVPGVGVP | |
| GGGVPGAGVPGVGVPGVGVPGVGVPGGGVPGAGVPGGGVPGVGVP | |
| GVGVPGGGVPGAGVPGVGVPGVGVPGVGVPGGGVPGAGVPGGGVP | |
| GVGVPGVGVPGGGVPGAGVPGVGVPGVGVPGVGVPGGGVPGAGVP | |
| GGGVPGVGVPGVGVPGGGVPGAGVPGVGVPGVGVPGVGVPGGGVP | |
| GAGVPGGGVPGWP | |
| HSDGTFSDLSKGMEEEAVRLHEWLKNGGPSSGAPPPSGSPAGSPTSTE | 37 |
| EGTSESATPESGPGTSTEPSEGSAPGSPAGSPTSTEEGTSTEPSEGSAPG | |
| TSTEPSEGSAPGTSESATPESGPGSEPATSGSETPGSEPATSGSETPGSPA | |
| GSPTSTEEGTSESATPESGPGTSTEPSEGSAPGTSTEPSEGSAPGSPAGSP | |
| TSTEEGTSTEPSEGSAPGTSTEPSEGSAPGTSESATPESGPGTSTEPSEGS | |
| APGTSESATPESGPGSEPATSGSETPGTSTEPSEGSAPGTSTEPSEGSAP | |
| GTSESATPESGPGTSESATPESGPGSPAGSPTSTEEGTSESATPESGPGS | |
| EPATSGSETPGTSESATPESGPGTSTEPSEGSAPGTSTEPSEGSAPGTSTE | |
| PSEGSAPGTSTEPSEGSAPGTSTEPSEGSAPGTSTEPSEGSAPGSPAGSP | |
| TSTEEGTSTEPSEGSAPGTSESATPESGPGSEPATSGSETPGTSESATPES | |
| GPGSEPATSGSETPGTSESATPESGPGTSTEPSEGSAPGTSESATPESGP | |
| GSPAGSPTSTEEGSPAGSPTSTEEGSPAGSPTSTEEGTSESATPESGPGT | |
| STEPSEGSAPGTSESATPESGPGSEPATSGSETPGTSESATPESGPGSEPA | |
| TSGSETPGTSESATPESGPGTSTEPSEGSAPGSPAGSPTSTEEGTSESATP | |
| ESGPGSEPATSGSETPGTSESATPESGPGSPAGSPTSTEEGSPAGSPTSTE | |
| EGTSTEPSEGSAPGTSESATPESGPGTSESATPESGPGTSESATPESGPG | |
| SEPATSGSETPGSEPATSGSETPGSPAGSPTSTEEGTSTEPSEGSAP | |
| HSDGTFSDLSKGMEEEAVRLHEWLKNGGPSSGAPPPSGTSTEPSEGSA | 38 |
| PGSEPATSGSETPGSPAGSPTSTEEGSTSSTAESPGPGTSTPESGSASPGS | |
| TSESPSGTAPGSTSESPSGTAPGTSTPESGSASPGTSTPESGSASPGSEPA | |
| TSGSETPGTSESATPESGPGSPAGSPTSTEEGTSTEPSEGSAPGTSESATP | |
| ESGPGTSTEPSEGSAPGTSTEPSEGSAPGSPAGSPTSTEEGTSTEPSEGS | |
| APGTSTEPSEGSAPGTSESATPESGPGTSESATPESGPGTSTEPSEGSAP | |
| GTSTEPSEGSAPGTSESATPESGPGTSTEPSEGSAPGSEPATSGSETPGSP | |
| AGSPTSTEEGSSTPSGATGSPGTPGSGTASSSPGSSTPSGATGSPGTSTE | |
| PSEGSAPGTSTEPSEGSAPGSEPATSGSETPGSPAGSPTSTEEGSPAGSP | |
| TSTEEGTSTEPSEGSAPGASASGAPSTGGTSESATPESGPGSPAGSPTST | |
| EEGSPAGSPTSTEEGSTSSTAESPGPGSTSESPSGTAPGTSPSGESSTAPG | |
| TPGSGTASSSPGSSTPSGATGSPGSSPSASTGTGPGSEPATSGSETPGTS | |
| ESATPESGPGSEPATSGSETPGSTSSTAESPGPGSTSSTAESPGPGTSPSG | |
| ESSTAPGSEPATSGSETPGSEPATSGSETPGTSTEPSEGSAPGSTSSTAES | |
| PGPGTSTPESGSASPGSTSESPSGTAPGTSTEPSEGSAPGTSTEPSEGSAP | |
| GTSTEPSEGSAPGSSTPSGATGSPGSSPSASTGTGPGASPGTSSTGSPGS | |
| EPATSGSETPGTSESATPESGPGGSPAGSPTSTEEGSSTPSGATGSPGSS | |
| PSASTGTGPGASPGTSSTSPGTSESATPESGPGTSTEPSEGSAPGTSTEPS | |
| EGSAP | |
| HSDGTFSDLSKGMEEEAVRLHEWLKNGGPSSGAPPPSVPGVGVPGVG | 39 |
| VPGGGVPGAGVPGVGVPGVGVPGVGVPGGGVPGAGVPGGGVPGVG | |
| VPGVGVPGGGVPGAGVPGVGVPGVGVPGVGVPGGGVPGAGVPGGG | |
| VPGVGVPGVGVPGGGVPGAGVPGVGVPGVGVPGVGVPGGGVPGAG | |
| VPGGGVPGVGVPGVGVPGGGVPGAGVPGVGVPGVGVPGVGVPGGG | |
| VPGAGVPGGGVPGVGVPGVGVPGGGVPGAGVPGVGVPGVGVPGVG | |
| VPGGGVPGAGVPGGGVPGVGVPGVGVPGGGVPGAGVPGVGVPGVG | |
| VPGVGVPGGGVPGAGVPGGGVPGVGVPGVGVPGGGVPGAGVPGVG | |
| VPGVGVPGVGVPGGGVPGAGVPGGGVPGVGVPGVGVPGGGVPGAG | |
| VPGVGVPGVGVPGVGVPGGGVPGAGVPGGGVPGVGVPGVGVPGGG | |
| VPGAGVPGVGVPGVGVPGVGVPGGGVPGAGVPGGGVPGVGVPGVG | |
| VPGGGVPGAGVPGVGVPGVGVPGVGVPGGGVPGAGVPGGGVPGVG | |
| VPGVGVPGGGVPGAGVPGVGVPGVGVPGVGVPGGGVPGAGVPGGG | |
| VPGVGVPGVGVPGGGVPGAGVPGVGVPGVGVPGVGVPGGGVPGAG | |
| VPGGGVPGWP | |
| HGEGTFTSDLSKQMEEEAVRLFIEWLKNGGPSSGAPPPSGSPAGSPTST | 40 |
| EEGTSESATPESGPGTSTEPSEGSAPGSPAGSPTSTEEGTSTEPSEGSAP | |
| GTSTEPSEGSAPGTSESATPESGPGSEPATSGSETPGSEPATSGSETPGSP | |
| AGSPTSTEEGTSESATPESGPGTSTEPSEGSAPGTSTEPSEGSAPGSPAG | |
| SPTSTEEGTSTEPSEGSAPGTSTEPSEGSAPGTSESATPESGPGTSTEPSE | |
| GSAPGTSESATPESGPGSEPATSGSETPGTSTEPSEGSAPGTSTEPSEGS | |
| APGTSESATPESGPGTSESATPESGPGSPAGSPTSTEEGTSESATPESGP | |
| GSEPATSGSETPGTSESATPESGPGTSTEPSEGSAPGTSTEPSEGSAPGT | |
| STEPSEGSAPGTSTEPSEGSAPGTSTEPSEGSAPGTSTEPSEGSAPGSPA | |
| GSPTSTEEGTSTEPSEGSAPGTSESATPESGPGSEPATSGSETPGTSESAT | |
| PESGPGSEPATSGSETPGTSESATPESGPGTSTEPSEGSAPGTSESATPES | |
| GPGSPAGSPTSTEEGSPAGSPTSTEEGSPAGSPTSTEEGTSESATPESGP | |
| GTSTEPSEGSAPGTSESATPESGPGSEPATSGSETPGTSESATPESGPGS | |
| EPATSGSETPGTSESATPESGPGTSTEPSEGSAPGSPAGSPTSTEEGTSES | |
| ATPESGPGSEPATSGSETPGTSESATPESGPGSPAGSPTSTEEGSPAGSP | |
| TSTEEGTSTEPSEGSAPGTSESATPESGPGTSESATPESGPGTSESATPES | |
| GPGSEPATSGSETPGSEPATSGSETPGSPAGSPTSTEEGTSTEPSEGSAP | |
| HGEGTFTSDLSKQMEEEAVRLFIEWLKNGGPSSGAPPPSGTSTEPSEGS | 41 |
| APGSEPATSGSETPGSPAGSPTSTEEGSTSSTAESPGPGTSTPESGSASP | |
| GSTSESPSGTAPGSTSESPSGTAPGTSTPESGSASPGTSTPESGSASPGSE | |
| PATSGSETPGTSESATPESGPGSPAGSPTSTEEGTSTEPSEGSAPGTSES | |
| ATPESGPGTSTEPSEGSAPGTSTEPSEGSAPGSPAGSPTSTEEGTSTEPSE | |
| GSAPGTSTEPSEGSAPGTSESATPESGPGTSESATPESGPGTSTEPSEGS | |
| APGTSTEPSEGSAPGTSESATPESGPGTSTEPSEGSAPGSEPATSGSETP | |
| GSPAGSPTSTEEGSSTPSGATGSPGTPGSGTASSSPGSSTPSGATGSPGT | |
| STEPSEGSAPGTSTEPSEGSAPGSEPATSGSETPGSPAGSPTSTEEGSPA | |
| GSPTSTEEGTSTEPSEGSAPGASASGAPSTGGTSESATPESGPGSPAGSP | |
| TSTEEGSPAGSPTSTEEGSTSSTAESPGPGSTSESPSGTAPGTSPSGESST | |
| APGTPGSGTASSSPGSSTPSGATGSPGSSPSASTGTGPGSEPATSGSETP | |
| GTSESATPESGPGSEPATSGSETPGSTSSTAESPGPGSTSSTAESPGPGTS | |
| PSGESSTAPGSEPATSGSETPGSEPATSGSETPGTSTEPSEGSAPGSTSST | |
| AESPGPGTSTPESGSASPGSTSESPSGTAPGTSTEPSEGSAPGTSTEPSEG | |
| SAPGTSTEPSEGSAPGSSTPSGATGSPGSSPSASTGTGPGASPGTSSTGS | |
| PGSEPATSGSETPGTSESATPESGPGGSPAGSPTSTEEGSSTPSGATGSP | |
| GSSPSASTGTGPGASPGTSSTSPGTSESATPESGPGTSTEPSEGSAPGTS | |
| TEPSEGSAP | |
| HGEGTFTSDLSKQMEEEAVRLFIEWLKNGGPSSGAPPPSVPGVGVPGV | 42 |
| GVPGGGVPGAGVPGVGVPGVGVPGVGVPGGGVPGAGVPGGGVPGV | |
| GVPGVGVPGGGVPGAGVPGVGVPGVGVPGVGVPGGGVPGAGVPGG | |
| GVPGVGVPGVGVPGGGVPGAGVPGVGVPGVGVPGVGVPGGGVPGA | |
| GVPGGGVPGVGVPGVGVPGGGVPGAGVPGVGVPGVGVPGVGVPGG | |
| GVPGAGVPGGGVPGVGVPGVGVPGGGVPGAGVPGVGVPGVGVPGV | |
| GVPGGGVPGAGVPGGGVPGVGVPGVGVPGGGVPGAGVPGVGVPGV | |
| GVPGVGVPGGGVPGAGVPGGGVPGVGVPGVGVPGGGVPGAGVPGV | |
| GVPGVGVPGVGVPGGGVPGAGVPGGGVPGVGVPGVGVPGGGVPGA | |
| GVPGVGVPGVGVPGVGVPGGGVPGAGVPGGGVPGVGVPGVGVPGG | |
| GVPGAGVPGVGVPGVGVPGVGVPGGGVPGAGVPGGGVPGVGVPGV | |
| GVPGGGVPGAGVPGVGVPGVGVPGVGVPGGGVPGAGVPGGGVPGV | |
| GVPGVGVPGGGVPGAGVPGVGVPGVGVPGVGVPGGGVPGAGVPGG | |
| GVPGVGVPGVGVPGGGVPGAGVPGVGVPGVGVPGVGVPGGGVPGA | |
| GVPGGGVPGWP | |
| HAEGTFTSKVSSYLEGQAAKEFIAWLVKGRGSPAGSPTSTEEGTSESA | 43 |
| TPESGPGTSTEPSEGSAPGSPAGSPTSTEEGTSTEPSEGSAPGTSTEPSEG | |
| SAPGTSESATPESGPGSEPATSGSETPGSEPATSGSETPGSPAGSPTSTEE | |
| GTSESATPESGPGTSTEPSEGSAPGTSTEPSEGSAPGSPAGSPTSTEEGT | |
| STEPSEGSAPGTSTEPSEGSAPGTSESATPESGPGTSTEPSEGSAPGTSES | |
| ATPESGPGSEPATSGSETPGTSTEPSEGSAPGTSTEPSEGSAPGTSESAT | |
| PESGPGTSESATPESGPGSPAGSPTSTEEGTSESATPESGPGSEPATSGSE | |
| TPGTSESATPESGPGTSTEPSEGSAPGTSTEPSEGSAPGTSTEPSEGSAP | |
| GTSTEPSEGSAPGTSTEPSEGSAPGTSTEPSEGSAPGSPAGSPTSTEEGT | |
| STEPSEGSAPGTSESATPESGPGSEPATSGSETPGTSESATPESGPGSEPA | |
| TSGSETPGTSESATPESGPGTSTEPSEGSAPGTSESATPESGPGSPAGSPT | |
| STEEGSPAGSPTSTEEGSPAGSPTSTEEGTSESATPESGPGTSTEPSEGSA | |
| PGTSESATPESGPGSEPATSGSETPGTSESATPESGPGSEPATSGSETPG | |
| TSESATPESGPGTSTEPSEGSAPGSPAGSPTSTEEGTSESATPESGPGSEP | |
| ATSGSETPGTSESATPESGPGSPAGSPTSTEEGSPAGSPTSTEEGTSTEPS | |
| EGSAPGTSESATPESGPGTSESATPESGPGTSESATPESGPGSEPATSGS | |
| ETPGSEPATSGSETPGSPAGSPTSTEEGTSTEPSEGSAP | |
| HAEGTFTSKVSSYLEGQAAKEFIAWLVKGRGTSTEPSEGSAPGSEPAT | 44 |
| SGSETPGSPAGSPTSTEEGSTSSTAESPGPGTSTPESGSASPGSTSESPSG | |
| TAPGSTSESPSGTAPGTSTPESGSASPGTSTPESGSASPGSEPATSGSETP | |
| GTSESATPESGPGSPAGSPTSTEEGTSTEPSEGSAPGTSESATPESGPGT | |
| STEPSEGSAPGTSTEPSEGSAPGSPAGSPTSTEEGTSTEPSEGSAPGTSTE | |
| PSEGSAPGTSESATPESGPGTSESATPESGPGTSTEPSEGSAPGTSTEPSE | |
| GSAPGTSESATPESGPGTSTEPSEGSAPGSEPATSGSETPGSPAGSPTST | |
| EEGSSTPSGATGSPGTPGSGTASSSPGSSTPSGATGSPGTSTEPSEGSAP | |
| GTSTEPSEGSAPGSEPATSGSETPGSPAGSPTSTEEGSPAGSPTSTEEGT | |
| STEPSEGSAPGASASGAPSTGGTSESATPESGPGSPAGSPTSTEEGSPAG | |
| SPTSTEEGSTSSTAESPGPGSTSESPSGTAPGTSPSGESSTAPGTPGSGTA | |
| SSSPGSSTPSGATGSPGSSPSASTGTGPGSEPATSGSETPGTSESATPESG | |
| PGSEPATSGSETPGSTSSTAESPGPGSTSSTAESPGPGTSPSGESSTAPGS | |
| EPATSGSETPGSEPATSGSETPGTSTEPSEGSAPGSTSSTAESPGPGTSTP | |
| ESGSASPGSTSESPSGTAPGTSTEPSEGSAPGTSTEPSEGSAPGTSTEPSE | |
| GSAPGSSTPSGATGSPGSSPSASTGTGPGASPGTSSTGSPGSEPATSGSE | |
| TPGTSESATPESGPGGSPAGSPTSTEEGSSTPSGATGSPGSSPSASTGTG | |
| PGASPGTSSTSPGTSESATPESGPGTSTEPSEGSAPGTSTEPSEGSAP | |
| HAEGTFTSKVSSYLEGQAAKEFIAWLVKGRVPGVGVPGVGVPGGGV | 45 |
| PGAGVPGVGVPGVGVPGVGVPGGGVPGAGVPGGGVPGVGVPGVGV | |
| PGGGVPGAGVPGVGVPGVGVPGVGVPGGGVPGAGVPGGGVPGVGV | |
| PGVGVPGGGVPGAGVPGVGVPGVGVPGVGVPGGGVPGAGVPGGGV | |
| PGVGVPGVGVPGGGVPGAGVPGVGVPGVGVPGVGVPGGGVPGAGV | |
| PGGGVPGVGVPGVGVPGGGVPGAGVPGVGVPGVGVPGVGVPGGGV | |
| PGAGVPGGGVPGVGVPGVGVPGGGVPGAGVPGVGVPGVGVPGVGV | |
| PGGGVPGAGVPGGGVPGVGVPGVGVPGGGVPGAGVPGVGVPGVGV | |
| PGVGVPGGGVPGAGVPGGGVPGVGVPGVGVPGGGVPGAGVPGVGV | |
| PGVGVPGVGVPGGGVPGAGVPGGGVPGVGVPGVGVPGGGVPGAGV | |
| PGVGVPGVGVPGVGVPGGGVPGAGVPGGGVPGVGVPGVGVPGGGV | |
| PGAGVPGVGVPGVGVPGVGVPGGGVPGAGVPGGGVPGVGVPGVGV | |
| PGGGVPGAGVPGVGVPGVGVPGVGVPGGGVPGAGVPGGGVPGVGV | |
| PGVGVPGGGVPGAGVPGVGVPGVGVPGVGVPGGGVPGAGVPGGGV | |
| PGWP | |
| DLSKQMEEEAVRLFIEWLKNGGPSSGAPPPSDAHKSEVAHRFKDLGE | 46 |
| ENFKALVLIAFAQYLQQCPFEDHVKLVNEVTEFAKTCVADESAENCD | |
| KSLHTLFGDKLCTVATLRETYGEMADCCAKQEPERNECFLQHKDDNP | |
| NLPRLVRPEVDVMCTAFHDNEETFLKKYLYEIARRHPYFYAPELLFFA | |
| KRYKAAFTECCQAADKAACLLPKLDELRDEGKASSAKQRLKCASLQ | |
| KFGERAFKAWAVARLSQRFPKAEFAEVSKLVTDLTKVHTECCHGDLL | |
| ECADDRADLAKYICENQDSISSKLKECCEKPLLEKSHCIAEVENDEMP | |
| ADLPSLAADFVESKDVCKNYAEAKDVFLGMFLYEYARRHPDYSVVL | |
| LLRLAKTYETTLEKCCAAADPHECYAKVFDEFKPLVEEPQNLIKQNCE | |
| LFEQLGEYKFQNALLVRYTKKVPQVSTPTLVEVSRNLGKVGSKCCKH | |
| PEAKRMPCAEDYLSVVLNQLCVLHEKTPVSDRVTKCCTESLVNRRPC | |
| FSALEVDETYVPKEFNAETFTFHADICTLSEKERQIKKQTALVELVKH | |
| KPKATKEQLKAVMDDFAAFVEKCCKADDKETCFAEEGKKLVAASQA | |
| ALGL | |
| DAHKSEVAHRFKDLGEENFKALVLIAFAQYLQQCPFEDHVKLVNEVT | 47 |
| EFAKTCVADESAENCDKSLHTLFGDKLCTVATLRETYGEMADCCAK | |
| QEPERNECFLQHKDDNPNLPRLVRPEVDVMCTAFHDNEETFLKKYLY | |
| EIARRHPYFYAPELLFFAKRYKAAFTECCQAADKAACLLPKLDELRDE | |
| GKASSAKQRLKCASLQKFGERAFKAWAVARLSQRFPKAEFAEVSKL | |
| VTDLTKVHTECCHGDLLECADDRADLAKYICENQDSISSKLKECCEKP | |
| LLEKSHCIAEVENDEMPADLPSLAADFVESKDVCKNYAEAKDVFLGM | |
| FLYEYARRHPDYSVVLLLRLAKTYETTLEKCCAAADPHECYAKVFDE | |
| FKPLVEEPQNLIKQNCELFEQLGEYKFQNALLVRYTKKVPQVSTPTLV | |
| EVSRNLGKVGSKCCKHPEAKRMPCAEDYLSVVLNQLCVLHEKTPVS | |
| DRVTKCCTESLVNRRPCFSALEVDETYVPKEFNAETFTFHADICTLSE | |
| KERQIKKQTALVELVKHKPKATKEQLKAVMDDFAAFVEKCCKADDK | |
| ETCFAEEGKKLVAASQAALGLDLSKQMEEEAVRLFIEWLKNGGPSSG | |
| APPPS | |
| TFTSDLSKQMEEEAVRLFIEWLKNGGPSSGAPPPSDAHKSEVAHRFKD | 48 |
| LGEENFKALVLIAFAQYLQQCPFEDHVKLVNEVTEFAKTCVADESAE | |
| NCDKSLHTLFGDKLCTVATLRETYGEMADCCAKQEPERNECFLQHK | |
| DDNPNLPRLVRPEVDVMCTAFHDNEETFLKKYLYEIARRHPYFYAPE | |
| LLFFAKRYKAAFTECCQAADKAACLLPKLDELRDEGKASSAKQRLKC | |
| ASLQKFGERAFKAWAVARLSQRFPKAEFAEVSKLVTDLTKVHTECCH | |
| GDLLECADDRADLAKYICENQDSISSKLKECCEKPLLEKSHCIAEVEN | |
| DEMPADLPSLAADFVESKDVCKNYAEAKDVFLGMFLYEYARRHPDY | |
| SVVLLLRLAKTYETTLEKCCAAADPHECYAKVFDEFKPLVEEPQNLIK | |
| QNCELFEQLGEYKFQNALLVRYTKKVPQVSTPTLVEVSRNLGKVGSK | |
| CCKHPEAKRMPCAEDYLSVVLNQLCVLHEKTPVSDRVTKCCTESLVN | |
| RRPCFSALEVDETYVPKEFNAETFTFHADICTLSEKERQIKKQTALVEL | |
| VKHKPKATKEQLKAVMDDFAAFVEKCCKADDKETCFAEEGKKLVA | |
| ASQAALGL | |
| GEGTFTSELSKQMEEEAVRLFIEWLKNGGPSSGAPPPSDAHKSEVAHR | 49 |
| FKDLGEENFKALVLIAFAQYLQQCPFEDHVKLVNEVTEFAKTCVADE | |
| SAENCDKSLHTLFGDKLCTVATLRETYGEMADCCAKQEPERNECFLQ | |
| HKDDNPNLPRLVRPEVDVMCTAFHDNEETFLKKYLYEIARRHPYFYA | |
| PELLFFAKRYKAAFTECCQAADKAACLLPKLDELRDEGKASSAKQRL | |
| KCASLQKFGERAFKAWAVARLSQRFPKAEFAEVSKLVTDLTKVHTEC | |
| CHGDLLECADDRADLAKYICENQDSISSKLKECCEKPLLEKSHCIAEV | |
| ENDEMPADLPSLAADFVESKDVCKNYAEAKDVFLGMFLYEYARRHP | |
| DYSVVLLLRLAKTYETTLEKCCAAADPHECYAKVFDEFKPLVEEPQN | |
| LIKQNCELFEQLGEYKFQNALLVRYTKKVPQVSTPTLVEVSRNLGKV | |
| GSKCCKHPEAKRMPCAEDYLSVVLNQLCVLHEKTPVSDRVTKCCTES | |
| LVNRRPCFSALEVDETYVPKEFNAETFTFHADICTLSEKERQIKKQTAL | |
| VELVKHKPKATKEQLKAVMDDFAAFVEKCCKADDKETCFAEEGKKL | |
| VAASQAALGL | |
| TFTSDLSKQMEEEAVRLFIEWLKNGGPSSGAPPPSGSPAGSPTSTEEGT | 50 |
| SESATPESGPGTSTEPSEGSAPGSPAGSPTSTEEGTSTEPSEGSAPGTSTE | |
| PSEGSAPGTSESATPESGPGSEPATSGSETPGSEPATSGSETPGSPAGSP | |
| TSTEEGTSESATPESGPGTSTEPSEGSAPGTSTEPSEGSAPGSPAGSPTST | |
| EEGTSTEPSEGSAPGTSTEPSEGSAPGTSESATPESGPGTSTEPSEGSAP | |
| GTSESATPESGPGSEPATSGSETPGTSTEPSEGSAPGTSTEPSEGSAPGT | |
| SESATPESGPGTSESATPESGPGSPAGSPTSTEEGTSESATPESGPGSEPA | |
| TSGSETPGTSESATPESGPGTSTEPSEGSAPGTSTEPSEGSAPGTSTEPSE | |
| GSAPGTSTEPSEGSAPGTSTEPSEGSAPGTSTEPSEGSAPGSPAGSPTST | |
| EEGTSTEPSEGSAPGTSESATPESGPGSEPATSGSETPGTSESATPESGP | |
| GSEPATSGSETPGTSESATPESGPGTSTEPSEGSAPGTSESATPESGPGSP | |
| AGSPTSTEEGSPAGSPTSTEEGSPAGSPTSTEEGTSESATPESGPGTSTEP | |
| SEGSAPGTSESATPESGPGSEPATSGSETPGTSESATPESGPGSEPATSG | |
| SETPGTSESATPESGPGTSTEPSEGSAPGSPAGSPTSTEEGTSESATPESG | |
| PGSEPATSGSETPGTSESATPESGPGSPAGSPTSTEEGSPAGSPTSTEEG | |
| TSTEPSEGSAPGTSESATPESGPGTSESATPESGPGTSESATPESGPGSEP | |
| ATSGSETPGSEPATSGSETPGSPAGSPTSTEEGTSTEPSEGSAP | |
| TFTSDLSKQMEEEAVRLFIEWLKNGGPSSGAPPPSGTSTEPSEGSAPGS | 51 |
| EPATSGSETPGSPAGSPTSTEEGSTSSTAESPGPGTSTPESGSASPGSTSE | |
| SPSGTAPGSTSESPSGTAPGTSTPESGSASPGTSTPESGSASPGSEPATSG | |
| SETPGTSESATPESGPGSPAGSPTSTEEGTSTEPSEGSAPGTSESATPESG | |
| PGTSTEPSEGSAPGTSTEPSEGSAPGSPAGSPTSTEEGTSTEPSEGSAPG | |
| TSTEPSEGSAPGTSESATPESGPGTSESATPESGPGTSTEPSEGSAPGTST | |
| EPSEGSAPGTSESATPESGPGTSTEPSEGSAPGSEPATSGSETPGSPAGS | |
| PTSTEEGSSTPSGATGSPGTPGSGTASSSPGSSTPSGATGSPGTSTEPSE | |
| GSAPGTSTEPSEGSAPGSEPATSGSETPGSPAGSPTSTEEGSPAGSPTST | |
| EEGTSTEPSEGSAPGASASGAPSTGGTSESATPESGPGSPAGSPTSTEEG | |
| SPAGSPTSTEEGSTSSTAESPGPGSTSESPSGTAPGTSPSGESSTAPGTPG | |
| SGTASSSPGSSTPSGATGSPGSSPSASTGTGPGSEPATSGSETPGTSESA | |
| TPESGPGSEPATSGSETPGSTSSTAESPGPGSTSSTAESPGPGTSPSGESS | |
| TAPGSEPATSGSETPGSEPATSGSETPGTSTEPSEGSAPGSTSSTAESPGP | |
| GTSTPESGSASPGSTSESPSGTAPGTSTEPSEGSAPGTSTEPSEGSAPGTS | |
| TEPSEGSAPGSSTPSGATGSPGSSPSASTGTGPGASPGTSSTGSPGSEPA | |
| TSGSETPGTSESATPESGPGGSPAGSPTSTEEGSSTPSGATGSPGSSPSAS | |
| TGTGPGASPGTSSTSPGTSESATPESGPGTSTEPSEGSAPGTSTEPSEGS | |
| AP | |
| TFTSDLSKQMEEEAVRLFIEWLKNGGPSSGAPPPSVPGVGVPGVGVPG | 52 |
| GGVPGAGVPGVGVPGVGVPGVGVPGGGVPGAGVPGGGVPGVGVPG | |
| VGVPGGGVPGAGVPGVGVPGVGVPGVGVPGGGVPGAGVPGGGVPG | |
| VGVPGVGVPGGGVPGAGVPGVGVPGVGVPGVGVPGGGVPGAGVPG | |
| GGVPGVGVPGVGVPGGGVPGAGVPGVGVPGVGVPGVGVPGGGVPG | |
| AGVPGGGVPGVGVPGVGVPGGGVPGAGVPGVGVPGVGVPGVGVPG | |
| GGVPGAGVPGGGVPGVGVPGVGVPGGGVPGAGVPGVGVPGVGVPG | |
| VGVPGGGVPGAGVPGGGVPGVGVPGVGVPGGGVPGAGVPGVGVPG | |
| VGVPGVGVPGGGVPGAGVPGGGVPGVGVPGVGVPGGGVPGAGVPG | |
| VGVPGVGVPGVGVPGGGVPGAGVPGGGVPGVGVPGVGVPGGGVPG | |
| AGVPGVGVPGVGVPGVGVPGGGVPGAGVPGGGVPGVGVPGVGVPG | |
| GGVPGAGVPGVGVPGVGVPGVGVPGGGVPGAGVPGGGVPGVGVPG | |
| VGVPGGGVPGAGVPGVGVPGVGVPGVGVPGGGVPGAGVPGGGVPG | |
| VGVPGVGVPGGGVPGAGVPGVGVPGVGVPGVGVPGGGVPGAGVPG | |
| GGVPGWP | |
| GEGTFTSELSKQMEEEAVRLFIEWLKNGGPSSGAPPPSGSPAGSPTSTE | 53 |
| EGTSESATPESGPGTSTEPSEGSAPGSPAGSPTSTEEGTSTEPSEGSAPG | |
| TSTEPSEGSAPGTSESATPESGPGSEPATSGSETPGSEPATSGSETPGSPA | |
| GSPTSTEEGTSESATPESGPGTSTEPSEGSAPGTSTEPSEGSAPGSPAGSP | |
| TSTEEGTSTEPSEGSAPGTSTEPSEGSAPGTSESATPESGPGTSTEPSEGS | |
| APGTSESATPESGPGSEPATSGSETPGTSTEPSEGSAPGTSTEPSEGSAP | |
| GTSESATPESGPGTSESATPESGPGSPAGSPTSTEEGTSESATPESGPGS | |
| EPATSGSETPGTSESATPESGPGTSTEPSEGSAPGTSTEPSEGSAPGTSTE | |
| PSEGSAPGTSTEPSEGSAPGTSTEPSEGSAPGTSTEPSEGSAPGSPAGSP | |
| TSTEEGTSTEPSEGSAPGTSESATPESGPGSEPATSGSETPGTSESATPES | |
| GPGSEPATSGSETPGTSESATPESGPGTSTEPSEGSAPGTSESATPESGP | |
| GSPAGSPTSTEEGSPAGSPTSTEEGSPAGSPTSTEEGTSESATPESGPGT | |
| STEPSEGSAPGTSESATPESGPGSEPATSGSETPGTSESATPESGPGSEPA | |
| TSGSETPGTSESATPESGPGTSTEPSEGSAPGSPAGSPTSTEEGTSESATP | |
| ESGPGSEPATSGSETPGTSESATPESGPGSPAGSPTSTEEGSPAGSPTSTE | |
| EGTSTEPSEGSAPGTSESATPESGPGTSESATPESGPGTSESATPESGPG | |
| SEPATSGSETPGSEPATSGSETPGSPAGSPTSTEEGTSTEPSEGSAP | |
| GEGTFTSELSKQMEEEAVRLFIEWLKNGGPSSGAPPPSGTSTEPSEGSA | 54 |
| PGSEPATSGSETPGSPAGSPTSTEEGSTSSTAESPGPGTSTPESGSASPGS | |
| TSESPSGTAPGSTSESPSGTAPGTSTPESGSASPGTSTPESGSASPGSEPA | |
| TSGSETPGTSESATPESGPGSPAGSPTSTEEGTSTEPSEGSAPGTSESATP | |
| ESGPGTSTEPSEGSAPGTSTEPSEGSAPGSPAGSPTSTEEGTSTEPSEGS | |
| APGTSTEPSEGSAPGTSESATPESGPGTSESATPESGPGTSTEPSEGSAP | |
| GTSTEPSEGSAPGTSESATPESGPGTSTEPSEGSAPGSEPATSGSETPGSP | |
| AGSPTSTEEGSSTPSGATGSPGTPGSGTASSSPGSSTPSGATGSPGTSTE | |
| PSEGSAPGTSTEPSEGSAPGSEPATSGSETPGSPAGSPTSTEEGSPAGSP | |
| TSTEEGTSTEPSEGSAPGASASGAPSTGGTSESATPESGPGSPAGSPTST | |
| EEGSPAGSPTSTEEGSTSSTAESPGPGSTSESPSGTAPGTSPSGESSTAPG | |
| TPGSGTASSSPGSSTPSGATGSPGSSPSASTGTGPGSEPATSGSETPGTS | |
| ESATPESGPGSEPATSGSETPGSTSSTAESPGPGSTSSTAESPGPGTSPSG | |
| ESSTAPGSEPATSGSETPGSEPATSGSETPGTSTEPSEGSAPGSTSSTAES | |
| PGPGTSTPESGSASPGSTSESPSGTAPGTSTEPSEGSAPGTSTEPSEGSAP | |
| GTSTEPSEGSAPGSSTPSGATGSPGSSPSASTGTGPGASPGTSSTGSPGS | |
| EPATSGSETPGTSESATPESGPGGSPAGSPTSTEEGSSTPSGATGSPGSS | |
| PSASTGTGPGASPGTSSTSPGTSESATPESGPGTSTEPSEGSAPGTSTEPS | |
| EGSAP | |
| GEGTFTSELSKQMEEEAVRLFIEWLKNGGPSSGAPPPSVPGVGVPGVG | 55 |
| VPGGGVPGAGVPGVGVPGVGVPGVGVPGGGVPGAGVPGGGVPGVG | |
| VPGVGVPGGGVPGAGVPGVGVPGVGVPGVGVPGGGVPGAGVPGGG | |
| VPGVGVPGVGVPGGGVPGAGVPGVGVPGVGVPGVGVPGGGVPGAG | |
| VPGGGVPGVGVPGVGVPGGGVPGAGVPGVGVPGVGVPGVGVPGGG | |
| VPGAGVPGGGVPGVGVPGVGVPGGGVPGAGVPGVGVPGVGVPGVG | |
| VPGGGVPGAGVPGGGVPGVGVPGVGVPGGGVPGAGVPGVGVPGVG | |
| VPGVGVPGGGVPGAGVPGGGVPGVGVPGVGVPGGGVPGAGVPGVG | |
| VPGVGVPGVGVPGGGVPGAGVPGGGVPGVGVPGVGVPGGGVPGAG | |
| VPGVGVPGVGVPGVGVPGGGVPGAGVPGGGVPGVGVPGVGVPGGG | |
| VPGAGVPGVGVPGVGVPGVGVPGGGVPGAGVPGGGVPGVGVPGVG | |
| VPGGGVPGAGVPGVGVPGVGVPGVGVPGGGVPGAGVPGGGVPGVG | |
| VPGVGVPGGGVPGAGVPGVGVPGVGVPGVGVPGGGVPGAGVPGGG | |
| VPGVGVPGVGVPGGGVPGAGVPGVGVPGVGVPGVGVPGGGVPGAG | |
| VPGGGVPGWP | |

### REFERENCES

1. Seino, S., and Miki, T. (2003) Prog. Biophys. Mol. Biol. 81, 133-176
2. Aguilar-Bryan, L., and Bryan, J. (1999) Endocr. Rev. 20, 101-135
3. Henquin, J. C. (2000) Diabetes 49, 1751-1760
4. Dunne, M. J., Cosgrove, K. E., Shepherd, R. M., Aynsley-Green, A., and Lindley, K. J. (2004) Physiol. Rev. 84, 239-275
5. Stanley, C. A. (2002) J. Clin. Endocrinol. Metab. 87, 4857-4859
6. Seghers, V., Nakazaki, M., DeMayo, F., Aguilar-Bryan, L., and Bryan, J. (2000) J. Biol. Chem. 275, 9270-9277
7. Shiota, C., Larsson, 0., Shelton, K. D., Shiota, M., Efanov, A. M., Hoy, M., Lindner, J., Kooptiwut, S., Juntti-Berggren, L., Gromada, J., Berggren, P. O., and Magnuson, M. A. (2002) J. Biol. Chem. 277, 37176-37183
8. Li, C., Buettger, C., Kwagh, J., Matter, A., Daikhin, Y., Nissim, I. B., Collins, H. W., Yudkoff, M., Stanley, C. A., and Matschinsky, F. M. (2004) J. Biol. Chem. 279, 13393-13401
9. Doliba, N. M., Wehrli, S. L., Vatamaniuk, M. Z., Qin, W., Buettger, C. W., Collins, H. W., and Matschinsky, F. M. (2007) Am. J. Physiol. 292, E1507-E1519
10. De León, D. D., Crutchlow, M. F., Ham, J. Y., and Stoffers, D. A. (2006) Int. J. Biochem. Cell Biol. 38, 845-859
11. Thorens, B. (1992) Proc. Natl. Acad. Sci. U.S.A. 89, 8641-8645
12. Gromada, J., Holst, J. J., and Rorsman, P. (1998) Pflugers Arch. 435, 583-594
13. Renstrom, E., Eliasson, L., and Rorsman, P. (1997) J. Physiol. 502, 105-118
14. Nakazaki, M., Crane, A., Hu, M., Seghers, V., Ullrich, S., Aguilar-Bryan, L., and Bryan, J. (2002) Diabetes 51, 3440-3449
15. Eliasson, L., Ma, X., Renstrom, E., Barg, S., Berggren, P. O., Galvanovskis, J., Gromada, J., Jing, X., Lundquist, I., Salehi, A., Sewing, S., and Rorsman, P. (2003) J. Gen. Physiol. 121, 181-197
16. Doliba, N. M., Qin, W., Vatamaniuk, M. Z., Li, C., Zelent, D., Najafi, H., Buettger, C. W., Collins, H. W., Carr, R. D., Magnuson, M. A., and Matschinsky, F. M. (2004) Am. J. Physiol. 286, E834-E843
17. Schirra, J., Sturm, K., Leicht, P., Arnold, R., Goke, B., and Katschinski, M. (1998) J. Clin. Invest. 101, 1421-1430
18. Kolligs, F., Fehmann, H. C., Goke, R., and Goke, B. (1995) Diabetes 44, 16-19
19. D'Alessio, D. A., Vogel, R., Prigeon, R., Laschansky, E., Koerker, D., Eng, J., and Ensinck, J. W. (1996) J. Clin. Invest. 97, 133-138
20. Wang, Z., Wang, R. M., Owji, A. A., Smith, D. M., Ghatei, M. A., and Bloom, S. R. (1995) J. Clin. Invest. 95, 417-421
21. Serre, V., Dolci, W., Schaerer, E., Scrocchi, L. A., Drucker, D. J., Efrat, S., and Thorens, B. (1998) Endocrinology 139, 4448-4454
22. De León, D. D., Deng, S., Madani, R., Ahima, R. A., Drucker, D. J., and Stoffers, D. A. (2003) Diabetes 52, 365-371
23. Gao, Z. Y., Li, G., Najafi, H., Wolf, B. A., and Matschinsky, F. M. (1999) Diabetes 48, 1535-1542
24. Edwards, C. M., Todd, J. F., Mahmoudi, M Wang, Z., Wang, R. M., Ghatei, M. A., and Bloom, S. R. (1999) Diabetes 48, 86-93
25. Pilkis, S. J., Claus, T. H., Johnson, R. A., and Park, C. R. (1975) J. Biol. Chem. 250, 6328-6336
26. Senft, G., Schultz, G., Munske, K., and Hoffmann, M. (1968) Diabetologia 4, 322-329
27. Loten, E. G., Assimacopoulos-Jeannet, F. D., Exton, J. H., and Park, C. R. (1978) J. Biol. Chem. 253, 746-757
28. Loten, E. G., and Sneyd, J. G. (1970) Biochem. J. 120, 187-193
29. Flamez, D., Gilon, P., Moens, K., Van Breusegem, A., Delmeire, D., Scrocchi, L. A., Henquin, J.-C., Drucker, D. J., and Schuit, F. (1999) Diabetes 48, 1979-1986
30. Shiota, C., Rocheleau, J. V., Shiota, M., Piston, D. W., and Magnuson, M. A. (2005) Am. J. Physiol. 289, E570-E577
31. Fourtner, S. H., Stanley, C. A., and Kelly, A. (2006) J. Pediatr. 149, 47-52
32. Masur, K., Tibaduiza, E. C., Chen, C., Ligon, B., and Beinborn, M. (2005) Mol. Endocrinol. 19, 1373-1382
33. Moens, K., Flamez, D., Van Schravendijk, C., Ling, Z., Pipeleers, D., and Schuit, F. (1998) Diabetes 47, 66-72
34. Holst, J. J., and Gromada, J. (2004) Am. J. Physiol. 287, E199-E206
35. Ozaki, N., Shibasaki, T., Kashima, Y., Miki, T., Takahashi, K., Ueno, H., Sunaga, Y., Yano, H., Matsuura, Y., Iwanaga, T., Takai, Y., and Seino, S. (2000) Nat. Cell Biol. 2, 805-811
36. Kashima, Y., Miki, T., Shibasaki, T., Ozaki, N., Miyazaki, M., Yano, H., and Seino, S. (2001) J. Biol. Chem. 276, 46046-46053
37. Shibasaki, T., Takahashi, H., Miki, T., Sunaga, Y., Matsumura, K., Yamanaka, M., Zhang, C., Tamamoto, A., Satoh, T., Miyazaki, J., and Seino, S. (2007) Proc. Natl. Acad. Sci. U.S.A. 104, 19333-19338
38. Ammala, C., Ashcroft, F. M., and Rorsman, P. (1993) Nature 363, 356-358
39. Yajima, H., Komatsu, M., Schennerhorn, T., Aizawa, T., Kaneko, T., Nagai, M., Sharp, G. W., and Hashizume, K. (1999) Diabetes 48, 1006-1012
40. Stanley C A. Advances in diagnosis and treatment of hyperinsulinism in infants and children. J Clin Endocrinol Metab 2002; 87:4867-4869
41. Beltrand J, Caquard M, Arnoux J B, et al. Glucose metabolism in 106 children and adolescents after pancreatectomy for congenital hyperinsulinism. Diabetes Care 2012; 36:198-203
42. Nauck M A. Unraveling the science of incretin biology. Am J Med 2009; 122 (Suppl.):S3-S10
43. D'Alessio D A, Vogel R, Prigeon R, et al. Elimination of the action of glucagon-like peptide 1 causes an impairment of glucose tolerance after nu-trient ingestion by healthy baboons. J Clin Invest 1996; 97:133-138
44. Edwards C M, Todd J F, Mahmoudi M, et al. Glucagon-like peptide 1 has a physiological role in the control of postprandial glucose in humans: studies with the antagonist exendin 9-39. Diabetes 1999; 48:86-93
45. Schirra J, Nicolaus M, Roggel R, et al. Endogenous glucagon-like peptide 1 controls endocrine pancreatic secretion and antro-pyloro-duodenal motility in humans. Gut 2006; 66:243-261
46. De León D D, Deng S, Madani R, Ahima R S, Drucker D J, Stoffers D A. Role of endogenous glucagon-like peptide-1 in islet regeneration after partial pancreatectomy. Diabetes 2003; 62:366-371
47. Hansotia T, Drucker D J. GIP and GLP-1 as incretin hormones: lessons from single and double incretin receptor knockout mice. Regul Pept 2006; 128: 126-134
48. Schirra J, Nicolaus M, Woerle H J, Struckmeier C, Katschinski M, Goke B. GLP-1 regulates gastroduodenal motility involving cholinergic pathways. Neurogastroenterol Motil 2009; 21:609-618
49. Nicolaus M, Brödl J, Lithe R, Woerle H J, Goke B, Schirra J. Endogenous GLP-1 regulates postprandial glycemia in humans: relative contributions of insulin, glucagon, and gastric emptying. J Clin Endocrinol Metab 2011; 96: 229-236
50. Palladino A A, Sayed S, Katz L E, Gallagher P R, De León D D. Increased glucagon-like peptide-1 secretion and postprandial hypoglycemia in children after Nissen fundoplication. J Clin Endocrinol Metab 2009; 94: 39-44
51. Schirra J, Sturm K, Leicht P, Arnold R, Goke B, Katschinski M. Exendin(9-39)amide is an antagonist of glucagon-like peptide-1(7-36)amide in hu-mans. J Clin Invest 1998; 101:1421-1430
52. De León D D, Li C, Delson M I, Matschinsky F M, Stanley C A, Stoffers D A. Exendin-(9-39) corrects fasting hypoglycemia in SUR-1−/− mice by lowering cAMP in pancreatic beta-cells and inhibiting insulin secretion. J Biol Chem 2008; 283:26786-26793
53. Li C, Buettger C, Kwagh J, et al. A signaling role of glutamine in insulin secretion. J Biol Chem 2004; 279:13393-13401
54. Rickels M R, Naji A. Reactive hypoglycaemia following GLP-1 infusion in pancreas transplant recipients. Diabetes Obes Metab 2010; 12:731-733
55. Toft-Nielsen M, Madsbad S, Holst J J. Exaggerated secretion of glucagon-like peptide-1 (GLP-1) could cause reactive hypoglycaemia. Diabetologia 1998; 41:1180-1186
56. Todd J F, Stanley S A, Roufosse C A, et al. A tumour that secretes glucagon-like peptide-1 and somatostatin in a patient with reactive hypoglycaemia and diabetes. Lancet 2003; 361:228-230
57. Kendall D M, Riddle M C, Rosenstock J, et al. Effects of exenatide (exendin-4) on glycemic control over 30 weeks in patients with type 2 diabetes treated with metformin and a sulfonylurea. Diabetes Care 2005; 28:1083-1091
58. Reimann F, Gribble F M. Glucose-sensing in glucagon-like peptide-1-secreting cells. Diabetes 2002; 51:2757-2763
59. Salehi M, Prigeon R L, D'Alessio D A. Gastric bypass surgery enhances glucagon-like peptide 1-stimulated postprandial insulin secretion in hu-mans. Diabetes 2011; 60:2308-2314
60. Serre V, Dolci W, Schaerer E, et al. Exendin-(9-39) is an inverse agonist of the murine glucagon-like peptide-1 receptor: implications for basal intracellular cyclic adenosine 3′,5′-monophosphate levels and beta-cell glucose competence. Endocrinology 1998; 139:4448-4454
61. Flamez D, Gilon P, Moens K, et al. Altered cAMP and Ca2+ signaling in mouse pancreatic islets with glucagon-like peptide-1 receptor null phenotype. Diabetes 1999; 48:1979-1986
62. Moens K, Flamez D, Van Schravendijk C, Ling Z, Pipeleers D, Schuit F. Dual glucagon recognition by pancreatic beta-cells via glucagon and glucagon-like peptide 1 receptors. Diabetes 1998; 47:66-72
63. Hvidberg A, Nielsen M T, Hilsted J, Orskov C, Holst J J. Effect of glucagon-like peptide-1 (proglucagon 78-107 amide) on hepatic glucose production in healthy man. Metabolism 1994; 43:104-108
64. Van Dijk G, Lindskog S, Holst J J, Steffens A B, Ahrén B. Effects of glucagon-like peptide-1 on glucose turnover in rats. Am J Physiol 1996; 270:E1015-E1021
65. Hussain K, Bryan J, Christesen H T, Brusgaard K, Aguilar-Bryan L. Serum glucagon counterregulatory hormonal response to hypoglycemia is blunted in congenital hyperinsulinism. Diabetes 2005; 54:2946-2951
66. Gromada J, Ma X, Hoy M, et al. ATP-sensitive K+ channel-dependent regulation of glucagon release and electrical activity by glucose in wild-type and SUR1−/− mouse alpha-cells. Diabetes 2004; 53(Suppl. 3):S181-5189
67. Shiota C, Rocheleau J V, Shiota M, Piston D W, Magnuson M A. Impaired glucagon secretory responses in mice lacking the type 1 sulfonylurea re-ceptor. Am J Physiol Endocrinol Metab 2005; 289:E570-E577
68. Thornton P S, Alter C A, Katz L E, Baker L, Stanley C A. Short- and long-term use of octreotide in the treatment of congenital hyperinsulinism. J Pediatr 1993; 123:637-643
69. Laje P, Halaby L, Adzick N S, Stanley C A. Necrotizing enterocolitis in ne-onates receiving octreotide for the management of congenital hyperinsulinism. Pediatr Diabetes 2010; 11:142-147
70. De León et al. Exendin-(9-39) Corrects Fasting Hypoglycemia in SUR-1^{-/-} Mice by Lowering cAMP in Pancreatic β-Cells and Inhibiting Insulin Secretion. J of Biological Chemistry 2008; 283(38):25786-25793
71. Calabria et al. GLP-1 Receptor Antagonist Exendin-(9-39) Elevates Fasting Blood Glucose Levels in Congenital Hyperinsulinism Owing to Inactivating Mutations in the ATP-Sensitive K⁺ Channel. Diabetes 2012; 61(10):2585-91

1. An isolated fusion protein, comprising an antagonist of the GLP-1 receptor (AGP) that is at least 90% identical to an amino acid sequence selected from Table 1, wherein said antagonist of the GLP-1 receptor is linked to a recombinant polypeptide (FPP), wherein the FPP is selected from Table 2

2. The isolated fusion protein of claim 1, wherein the antagonist of the GLP-1 receptor is selected from Seq ID No. 1, Seq ID No. 15 and Seq ID No. 18.

3. The isolated fusion protein of claim 1, wherein the antagonist peptide of GLP-1 receptor and the FPP are linked via a spacer, wherein the spacer sequence comprises between 1 to about 50 amino acid residues, and wherein the spacer optionally comprises a cleavage sequence.

4. The isolated fusion protein of claim 1, wherein the fusion protein binds to the same target receptor of the corresponding native antagonist of the GLP-1 receptor peptide that lacks the FPP, and wherein said fusion protein retains at least about 0.1% to about 30% or greater of the binding affinity of the corresponding antagonist peptide of the GLP-1 receptor that lacks the FPP.

5. The isolated fusion protein of claim 1, comprising an amino acid sequence that has at least 90% sequence identity to an amino acid sequence selected from Table 3.

6. A pharmaceutical composition comprising the isolated fusion protein of claim 1 and a pharmaceutically acceptable carrier.

7. The isolated protein of claim 1 that is configured according to formula I: (FPP)x-AGP-(FPP)y wherein independently for each occurrence: (a) x is either 0 or 1; and (b) y is either 0 or 1, wherein x+y>1.

8. The isolated fusion protein of claim 1, wherein the FPP is fused to an antagonist of the GLP-1 receptor peptide on an N- or C-terminus of the antagonist GLP-1 receptor peptide

9. The isolated fusion protein of claim 1, characterized in that: (i) it has a longer terminal half-life when administered to a subject compared to the corresponding antagonist peptide of GLP-1 receptor that lacks the FPP when administered to a subject at a comparable molar dose; (ii) when a smaller molar amount of the fusion protein is administered to a subject in comparison to the corresponding antagonist peptide of GLP-1 receptor that lacks the FPP administered to a subject under an otherwise equivalent dose regimen, the fusion protein achieves a comparable area under the curve (AUC) as the corresponding antagonist peptide of GLP-1 receptor that lacks the FPP; (iii) when a smaller molar amount of the fusion protein is administered to a subject in comparison to the corresponding antagonist peptide of GLP-1 receptor that lacks the FPP administered to a subject under an otherwise equivalent dose regimen, the fusion protein achieves a comparable therapeutic effect as the corresponding antagonist peptide of GLP-1 receptor that lacks the FPP; (iv) when the fusion protein is administered to a subject less frequently in comparison to the corresponding antagonist peptide of GLP-1 receptor that lacks the FPP administered to a subject using an otherwise equivalent molar amount, the fusion protein achieves a comparable area under the curve (AUC) as the corresponding antagonist peptide of GLP-1 receptor that lacks the FPP; (v) when the fusion protein is administered to a subject less frequently in comparison to the corresponding antagonist peptide of GLP-1 receptor that lacks the FPP administered to a subject using an otherwise equivalent molar amount, the fusion protein achieves a comparable therapeutic effect as the corresponding antagonist peptide of GLP-1 receptor that lacks the FPP; (vi) when an accumulatively smaller molar amount of the fusion protein is administered to a subject in comparison to the corresponding antagonist peptide of GLP-1 receptor that lacks the FPP administered to a subject under an otherwise equivalent dose period, the fusion protein achieves comparable area under the curve (AUC) as the corresponding antagonist peptide of GLP-1 receptor that lacks the FPP; or (vii) when an accumulatively smaller molar amount of the fusion protein is administered to a subject in comparison to the corresponding antagonist peptide of GLP-1 receptor that lacks the FPP administered to a subject under an otherwise equivalent dose period, the fusion protein achieves comparable therapeutic effect as the corresponding antagonist peptide of GLP-1 receptor that lacks the FPP.

10. A method of producing a fusion protein comprising a antagonist peptide of GLP-1 receptor fused to one or more recombinant polypeptides (FPP), comprising: (a) providing host cell comprising a recombinant polynucleotide molecule encoding the fusion protein of claim 1; (b) culturing the host cell under conditions permitting the expression of the fusion protein; and (c) recovering the fusion protein.

11. The method of claim 10, wherein the antagonist peptide of GLP-1 receptor of the fusion protein has at least 90% sequence identity to: (a) human antagonist peptide of GLP-1 receptor; or (b) a sequence selected from Table 1.

12. The method of claim 10, wherein the one or more FPP of the expressed fusion protein has at least 90%> sequence identity to a sequence selected from Table 2.

13. The method of claim 10, wherein the polynucleotide molecule encoding the fusion protein comprises a nucleic acid sequence exhibiting at least 90%>sequence identity of a nucleic acid of the peptides listed in Table 3.

14. The method of claim 13, wherein the polynucleotide is codon optimized for enhanced expression of said fusion protein in the host cell.

15. The method of claim 10, wherein the host cell is a prokaryotic cell.

16. The method of claim 10, wherein the isolated fusion protein is recovered from the host cell cytoplasm in substantially soluble form.

17. An isolated nucleic acid comprising a nucleotide sequence encoding the fusion protein of claim 1 or the complement thereof.

18. A method of treating a glucose regulating peptide related condition in a subject, comprising administering to the subject a therapeutically effective amount of a fusion protein of claim 1.

19. The method of claim 18, wherein the glucose regulating peptide related condition is selected from neonatal hyperinsulinism, congential hyperinsulinism, acute hypoglycemia, nocturnal hypoglycemia, chronic hypoglycemia, Beckwith-Wiedemann syndrome, congenital disorders of glycosylation, hypoglycemia resulting from dialysis, glucagonomas, secretory disorders of the airway, arthritis, neuroendocrine tumors, osteoporosis, central nervous system disease, restenosis, neurodegenerative disease, renal failure, congestive heart failure, nephrotic syndrome, cirrhosis, pulmonary edema, hypertension, and disorders wherein the reduction of food intake is desired, stroke, irritable bowel syndrome, myocardial infarction (e.g., reducing the morbidity and/or mortality associated therewith), stroke, acute coronary syndrome (e.g., characterized by an absence of Q-wave) myocardial infarction, post-surgical catabolic changes, hibernating myocardium or diabetic cardiomyopathy, post-prandial hypoglycemia, insufficient urinary sodium excretion, excessive urinary potassium concentration, conditions or disorders associated with toxic hypervolemia, (e.g., renal failure, congestive heart failure, nephrotic syndrome, cirrhosis, pulmonary edema, and hypertension), polycystic ovary syndrome, respiratory distress, nephropathy, left ventricular systolic dysfunction, (e.g., with abnormal left ventricular ejection fraction), gastrointestinal disorders such as diarrhea, postoperative dumping syndrome and irritable bowel syndrome, (i.e., via inhibition of antro-duodenal motility), critical illness polyneuropathy (CIPN), dyslipidemia, organ tissue injury caused by reperfusion of blood flow following ischemia, and coronary heart disease risk factor (CHDRF) syndrome.

20. The method of claim 18, wherein the therapeutically effective amount results in maintaining blood concentrations of the fusion protein within a therapeutic window for the fusion protein at least three-fold longer compared to the corresponding native antagonist peptide of GLP-1 receptor that lacks the FPP administered at a comparable amount to a subject.

21. The method of claim 20, wherein administration of two or more consecutive doses of the fusion protein administered using a therapeutically effective dose regimen to a subject results in a gain in time between consecutive Cmax peaks and/or Cmin troughs for blood levels of the fusion protein compared to the corresponding antagonist peptide of GLP-1 receptor not linked to the fusion protein and administered using a therapeutically dose regimen established for the AGP.

22. The method of claim 21, wherein (i) a smaller molar amount of the fusion protein is administered to a subject in comparison to the corresponding antagonist peptide of GLP-1 receptor that lacks the FPP administered to a subject under an otherwise equivalent dose regimen, and the fusion protein achieves a comparable therapeutic effect as the corresponding antagonist peptide of GLP-1 receptor that lacks the FPP; (ii) the fusion protein is administered less frequently to a subject in comparison to the corresponding antagonist peptide of GLP-1 receptor that lacks the FPP administered to a subject using an otherwise equivalent molar dose, and the fusion protein achieves a comparable therapeutic effect as the corresponding antagonist peptide of GLP-1 receptor that lacks the FPP; or (iii) an accumulatively smaller molar amount of the fusion protein is administered to a subject in comparison to the corresponding antagonist peptide of GLP-1 receptor that lacks the FPP administered to a subject under the otherwise equivalent dose period, the fusion protein achieves a comparable therapeutic effect as the corresponding antagonist peptide of GLP-1 receptor that lacks the FPP.

23. The method of claim 19, wherein the therapeutic effect is a measured parameter selected from HbAlc concentrations, insulin concentrations, stimulated C peptide, fasting plasma glucose (FPG), serum cytokine levels, CRP levels, insulin secretion and Insulin-sensitivity index derived from an oral glucose tolerance test (OGTT), body weight, and food consumption.
